SEQUENCE LISTING (43 sequences)

SEQ ID NO: 1 - DNA, 2740 bp, Homo sapiens
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgtccaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccgcgctgcgggcgcgcactccctgtttctcctcctcctggctggcgctgcctgcctctccgcactcactgctcgcgccgggcgcgctccgccagctccgtgctccccgcgccaccctcctccgggccgcgctccctaagggatggtactgaatttcgccgccacaggagaccggctggagcgcccgccccgcggcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccatgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagaaagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcaggggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgtttttttttttttttttttacaccataaagtgattattaagctttcctttttactctttggctagctttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttctttttttgttgatccaaaattaaaaaaaaaaataccaccaacaaaa

SEQ ID NO: 2 - DNA, 2809 bp, Homo sapiens
acgcgcgccctgcggagcccgcccaactccggcgagccgggcctgcgcctactcctcctcctcctctcccggcggcggctgcggcggaggcgccgactcggccttgcgcccgccctcaggcccgcgcgggcggcgcagcgaggccccgggcggcgggtggtggctgccaggcggctcggccgcgggcgctgcccggccccggcgagcggagggcggagcgcggcgccggagccgagggcgcgccgcggagggggtgctgggccgcgctgtgcccggccgggcggcggctgcaagaggaggccggaggcgagcgcggggccggcggtgggcgcgcagggcggctcgcagctcgcagccggggccgggccaggcgtccaggcaggtgatcggtgtggcggcggcggcggcggcggccccagactccctccggagttcttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccgcgctgcgggcgcgcactccctgtttctcctcctcctggctggcgctgcctgcctctccgcactcactgctcgcgccgggcgcgctccgccagctccgtgctccccgcgccaccctcctccgggccgcgctccctaagggatggtactgaatttcgccgccacaggagaccggctggagcgcccgccccgcggcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccatgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacgggaaggcctagggagtcaggtaaaaaacggaaaagaaaaaggttaaaacccacctaaagcagccaaccagatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagaaagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcaggggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgtttttttttttttttttttacaccataaagtgattattaagctttcctttttactctttggctagctttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttctttttttgttgatccaaaattaaaaaaaaaaataccaccaacaaaa

SEQ ID NO: 3 - DNA, 2701 bp, Homo sapiens
agagagagagagagactgactgagcaggaatggtgagatgtttatcatgggcctcggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggtccttggagtgataatattgtttccctcgtccgtctgtctcgatgcctgattcggacggccaatggtgcttcccccacccctccacgtgtccgtccacccttccatcagcgggtctcctcccagcggcctccggcgtcttgcccagcagctcaagaagaaaaagaaggactgaactccatcgccatcttcttcccttaactccaagaacttgggataagagtgtgagagagactgatggggtcgctctttgggggaaacgggctccttcccctgcacctggcctgggccacacctgagcgctgtggactgtcctgaggagccctgaggacctctcagcatagcctgcctgatccctgaacccctggccagctctgaggggaggcacctccaggcaggccaggctgcctcggactccatggctaagaccacagacgggcacacagactggagaaaacccctcccacggtgcccaaacaccagtcacctcgtctccctggtgcctctgtgcacagtggcttcttttcgttttcgttttgaagacgtggactcctcttggtgggtgtggccagcacaccaagtggctgggtgccctctcaggtgggttagagatggagtttgctgttgaggtggctgtagatggtgacctgggtatcccctgcctcctgccaccccttcctccccacactccactctgattcacctcttcctctggttcctttcatctctctacctccaccctgcattttcctcttgtcctggcccttcagtctgctccaccaaggggctcttgaaccccttattaaggccccagatgatcccagtcactcctctctagggcagaagactagaggccagggcagcaagggacctgctcatcatattccaacccagccacgactgccatgtaaggttgtgcagggtgtgtactgcacaaggacattgtatgcagggagcactgttcacatcatagataaagctgatttgtatatttattatgacaatttctggcagatgtaggtaaagaggaaaaggatccttttcctaattcacacaaagactccttgtggactggctgtgcccctgatgcagcctgtggcttggagtggccaaataggagggagactgtggtaggggcagggaggcaacactgctgtccacatgacctccatttcccaaagtcctctgctccagcaactgcccttccaggtgggtgtgggacacctgggagaaggtctccaagggagggtgcagccctcttgcccgcacccctccctgcttgcacacttccccatctttgatccttctgagctccacctctggtggctcctcctaggaaaccagctcgtgggctgggaatgggggagagaagggaaaagatccccaagaccccctggggtgggatctgagctcccacctcccttcccacctactgcactttcccccttcccgccttccaaaacctgcttccttcagtttgtaaagtcggtgattatatttttgggggctttccttttattttttaaatgtaaaatttatttatattccgtatttaaagttgtaaaaaaaaataaccacaaaacaaaaccaaatgaatccgccggaggtctgtctgttggcatcgtgcgtgacaattaacctttctgccttggcaggatgtgccgacagcttgcggcgtgttcctctcactctgggagcctcaggcgtgatctcacacactggcgtgcacatacacacacacacacatacatgctcacacatgcgtgcacatacacgcaggcctgcaacttgggggaggcctctgtctggcgggaagaagagacacacaggctactctgttggtcttggtcctggcacagctcctgacacgtggacttgtgcgtgtctctggcagtgacgagagatgggtttctgcag

SEQ ID NO: 4 - DNA, 18 bp, Artificial Sequence
  Synthetic Polynucleotide
tgggagtagatgaggtac

SEQ ID NO: 5 - DNA, 3801 bp, Homo sapiens
gaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggccccgcgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctcactcgggctcgggatccgcccaggtagcggcctcggacccaggtcctgcgcccaggtcctcccctgccccccagcgacggagccggggccgggggcggcggcgcccgggggccatgcgggtgagccgcggctgcagaggcctgagcgcctgatcgccgcggacccgagccgagcccacccccctccccagccccccaccctggccgcgggggcggcgcgctcgatctacgcgtccggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggtccttggagtgataatattgtttccctcgtccgtctgtctcgatgcctgattcggacggccaatggtgcttcccccacccctccacgtgtccgtccacccttccatcagcgggtctcctcccagcggcctccggcgtcttgcccagcagctcaagaagaaaaagaaggactgaactccatcgccatcttcttcccttaactccaagaacttgggataagagtgtgagagagactgatggggtcgctctttgggggaaacgggctccttcccctgcacctggcctgggccacacctgagcgctgtggactgtcctgaggagccctgaggacctctcagcatagcctgcctgatccctgaacccctggccagctctgaggggaggcacctccaggcaggccaggctgcctcggactccatggctaagaccacagacgggcacacagactggagaaaacccctcccacggtgcccaaacaccagtcacctcgtctccctggtgcctctgtgcacagtggcttcttttcgttttcgttttgaagacgtggactcctcttggtgggtgtggccagcacaccaagtggctgggtgccctctcaggtgggttagagatggagtttgctgttgaggtggctgtagatggtgacctgggtatcccctgcctcctgccaccccttcctccccacactccactctgattcacctcttcctctggttcctttcatctctctacctccaccctgcattttcctcttgtcctggcccttcagtctgctccaccaaggggctcttgaaccccttattaaggccccagatgatcccagtcactcctctctagggcagaagactagaggccagggcagcaagggacctgctcatcatattccaacccagccacgactgccatgtaaggttgtgcagggtgtgtactgcacaaggacattgtatgcagggagcactgttcacatcatagataaagctgatttgtatatttattatgacaatttctggcagatgtaggtaaagaggaaaaggatccttttcctaattcacacaaagactccttgtggactggctgtgcccctgatgcagcctgtggcttggagtggccaaataggagggagactgtggtaggggcagggaggcaacactgctgtccacatgacctccatttcccaaagtcctctgctccagcaactgcccttccaggtgggtgtgggacacctgggagaaggtctccaagggagggtgcagccctcttgcccgcacccctccctgcttgcacacttccccatctttgatccttctgagctccacctctggtggctcctcctaggaaaccagctcgtgggctgggaatgggggagagaagggaaaagatccccaagaccccctggggtgggatctgagctcccacctcccttcccacctactgcactttcccccttcccgccttccaaaacctgcttccttcagtttgtaaagtcggtgattatatttttgggggctttccttttattttttaaatgtaaaatttatttatattccgtatttaaagttgtaaaaaaaaataaccacaaaacaaaaccaaatgaatccgccggaggtctgtctgttggcatcgtgcgtgacaattaacctttctgccttggcaggatgtgccgacagcttgcggcgtgttcctctcactctgggagcctcaggcgtgatctcacacactggcgtgcacatacacacacacacacatacatgctcacacatgcgtgcacatacacgcaggcctgcaacttgggggaggcctctgtctggcgggaagaagagacacacaggctactctgttggtcttggtcctggcacagctcctgacacgtggacttgtgcgtgtctctggcagtgacgagagatgggtttctgcag

SEQ ID NO: 6 - DNA, 3016 bp, Homo sapiens
gcccggagagccgcatctattggcagctttgttattgatcagaaactgctcgccgccgacttggcttccagtctggctgcgggcaacccttgagttttcgcctctgtcctgtcccccgaactgacaggtgctcccagcaacttgctggggacttctcgccgctcccccgcgtccccaccccctcattcctccctcgccttcacccccacccccaccacttcgccacagctcaggatttgtttaaaccttgggaaactggttcaggtccaggttttgctttgatccttttcaaaaactggagacacagaagagggctctaggaaaaagttttggatgggattatgtggaaactaccctgcgattctctgctgccagagcaggctcggcgcttccaccccagtgcagccttcccctggcggtggtgaaagagactcgggagtcgctgcttccaaagtgcccgccgtgagtgagctctcaccccagtcagccaaatgagcctcttcgggcttctcctgctgacatctgccctggccggccagagacaggggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcgtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatgatataaaatattgttgctctgacaaaaatacatgtatttcattctcgtatggtgctagagttagattaatctgcattttaaaaaactgaattggaatagaattggtaagttgcaaagactttttgaaaataattaaattatcatatcttccattcctgttattggagatgaaaataaaaagcaacttatgaaagtagacattcagatccagccattactaacctattccttttttggggaaatctgagcctagctcagaaaaacataaagcaccttgaaaaagacttggcagcttcctgataaagcgtgctgtgctgtgcagtaggaacacatcctatttattgtgatgttgtggttttattatcttaaactctgttccatacacttgtataaatacatggatatttttatgtacagaagtatgtctcttaaccagttcacttattgtactctggcaatttaaaagaaaatcagtaaaatattttgcttgtaaaatgcttaatatcgtgcctaggttatgtggtgactatttgaatcaaaaatgtattgaatcatcaaataaaagaatgtggctattttggggagaaaattaaaaaaaaaaa

SEQ ID NO: 7 - DNA, 3995 bp, Homo sapiens
tctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccacaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaactaaaaa

SEQ ID NO: 8 - DNA, 3979 bp, Homo sapiens
tctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaactaaaaa

SEQ ID NO: 9 - DNA, 18 bp, Artificial Sequence
  Synthetic Polynucleotide
  misc_feature (1)..(18): modified with phosphorothioate bond
tgggagtagatgaggtac

SEQ ID NO: 10 - RNA, 18 nt, Artificial Sequence
  Synthetic Polynucleotide
  misc_feature (1)..(18): modified with O-methyl
ugggaguagaugagguac

SEQ ID NO: 11 - DNA, 6572 bp, Homo sapiens
aagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggatgacatcggcatagactcttcagacctggtggaagacagcttcctgtaactggcggattcgaggggttccttccacttctggggccacctctggatcccgttcagaaaaccactttattgcaatgcagaggttgagaggaggacttggttgatgtttaaagagaagttcccagccaagggcctcggggagcgttctaaatatgaatgaatgggatattttgaaatgaactttgtcagtgttgcctcttgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttttcttcatgcctgatgaaagctttggcgaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggcattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgtgtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtta

SEQ ID NO: 12 - DNA, 3278 bp, Homo sapiens
aatagactaaacccagagcctcaaagcagtgcactccgtgaaggcaaagagaacacgctgcaaaaggctttccaagaatcctcgacatggcaaggaggagctcgttccagtcgtgtcagataatatccctgttcacttttgccgttggagtcaatatctgcttaggattcactgcacatcgaattaagagagcagaaggatgggaggaaggtcctcctacagtgctatcagactccccctggaccaacatctccggatcttgcaagggcaggtgctttgaacttcaagaggctggacctcctgattgtcgctgtgacaacttgtgtaagagctataccagttgctgccatgactttgatgagctgtgtttgaagacagcccgtggctgggagtgtactaaggacagatgtggagaagtcagaaatgaagaaaatgcctgtcactgctcagaggactgcttggccaggggagactgctgtaccaattaccaagtggtttgcaaaggagagtcgcattgggttgatgatgactgtgaggaaataaaggccgcagaatgccctgcagggtttgttcgccctccattaatcatcttctccgtggatggcttccgtgcatcatacatgaagaaaggcagcaaagtcatgcctaatattgaaaaactaaggtcttgtggcacacactctccctacatgaggccggtgtacccaactaaaacctttcctaacttatacactttggccactgggctatatccagaatcacatggaattgttggcaattcaatgtatgatcctgtatttgatgccacttttcatctgcgagggcgagagaaatttaatcatagatggtggggaggtcaaccgctatggattacagccaccaagcaaggggtgaaagctggaacattcttttggtctgttgtcatccctcacgagcggagaatattaaccatattgcagtggctcaccctgccagatcatgagaggccttcggtctatgccttctattctgagcaacctgatttctctggacacaaatatggccctttcggccctgaggagagtagttatggctcaccttttactccggctaagagacctaagaggaaagttgcccctaagaggagacaggaaagaccagttgctcctccaaagaaaagaagaagaaaaatacataggatggatcattatgctgcggaaactcgtcaggacaaaatgacaaatcctctgagggaaatcgacaaaattgtggggcaattaatggatggactgaaacaactaaaactgcatcggtgtgtcaacgtcatctttgtcggagaccatggaatggaagatgtcacatgtgatagaactgagttcttgagtaattacctaactaatgtggatgatattactttagtgcctggaactctaggaagaattcgatccaaatttagcaacaatgctaaatatgaccccaaagccattattgccaatctcacgtgtaaaaaaccagatcagcactttaagccttacttgaaacagcaccttcccaaacgtttgcactatgccaacaacagaagaattgaggatatccatttattggtggaacgcagatggcatgttgcaaggaaacctttggatgtttataagaaaccatcaggaaaatgctttttccagggagaccacggatttgataacaaggtcaacagcatgcagactgtttttgtaggttatggctcaacatttaagtacaagactaaagtgcctccatttgaaaacattgaactttacaatgttatgtgtgatctcctgggattgaagccagctcctaataatgggacccatggaagtttgaatcatctcctgcgcactaataccttcaggccaaccatgccagaggaagttaccagacccaattatccagggattatgtaccttcagtctgattttgacctgggctgcacttgtgatgataaggtagagccaaagaacaagttggatgaactcaacaaacggcttcatacaaaagggtctacagaagagagacacctcctctatgggcgacctgcagtgctttatcggactagatatgatatcttatatcacactgactttgaaagtggttatagtgaaatattcctaatgccactctggacatcatatactgtttccaaacaggctgaggtttccagcgttcctgaccatctgaccagttgcgtccggcctgatgtccgtgtttctccgagtttcagtcagaactgtttggcctacaaaaatgataagcagatgtcctacggattcctctttcctccttatctgagctcttcaccagaggctaaatatgatgcattccttgtaaccaatatggttccaatgtatcctgctttcaaacgggtctggaattatttccaaagggtattggtgaagaaatatgcttcggaaagaaatggagttaacgtgataagtggaccaatcttcgactatgactatgatggcttacatgacacagaagacaaaataaaacagtacgtggaaggcagttccattcctgttccaactcactactacagcatcatcaccagctgtctggatttcactcagcctgccgacaagtgtgacggccctctctctgtgtcctccttcatcctgcctcaccggcctgacaacgaggagagctgcaatagctcagaggacgaatcaaaatgggtagaagaactcatgaagatgcacacagctagggtgcgtgacattgaacatctcaccagcctggacttcttccgaaagaccagccgcagctacccagaaatcctgacactcaagacatacctgcatacatatgagagcgagatttaactttctgagcatctgcagtacagtcttatcaactggttgtatatttttatattgtttttgtatttattaatttgaaaccaggacattaaaaatgttagtattttaatcctgtaccaaatctgacatattatgcctgaatgactccactgtttttctctaatgcttgatttaggtagccttgtgttctgagtagagcttgtaataaatactgcagcttgagtttttagtggaagcttctaaatggtgctgcagatttgatatttgcattgaggaaatattaattttccaatgcacagttgccacatttagtcctgtactgtatggaaacactgattttgtaaagttgcctttatttgctgttaactgttaactatgacagatatatttaagccttataaaccaatcttaaacataataaatcacacattcagttttttctggttt

SEQ ID NO: 13 - DNA, 1923 bp, Homo sapiens
acttctgccttgttatcagaggagcgaggatgtggcctgtgtgccgcctaatcatgcctagaactgtctgcacctgctgatcttttgcttctgcgaactggctgcacctgctgttctcctgctcataccctagcccgtgcttaccctaattccctgttctcctgcctcaccaccagaggcccgggttgtctgcacagtctcattttccacgttattcctgtaatgaacccgtcctcccacccccaccagcagttacagggctgggtgcggagaccaactctggccagacacagaggagcagcaagaacccggggcaacggtcctgtgaaaaccgaatcctcccgcttcggcgtcccaggagcccgcccctcgcccgccgcctcccctgcgtccccgccccgcgcagccgccgcgctcctgcgctccgaggtccgaggttcccgagatgaaggtctggctgctgcttggtcttctgctggtgcacgaagcgctggaggatgttactggccaacaccttcccaagaacaagcgtccaaaagaaccaggagagaatagaatcaaacctaccaacaagaaggtgaagcccaaaattcctaaaatgaaggacagggactcagccaattcagcaccaaagacgcagtctatcatgatgcaagtgctggataaaggtcgcttccagaaacccgccgctaccctgagtctgctggcggggcaaactgtagagcttcgatgtaaagggagtagaattgggtggagctaccctgcgtatctggacacctttaaggattctcgcctcagcgtcaagcagaatgagcgctacggccagttgactctggtcaactccacctcggcagacacaggtgaattcagctgctgggtgcagctctgcagcggctacatctgcaggaaggacgaggccaaaacgggctccacctacatcttttttacagagaaaggagaactctttgtaccttctcccagctacttcgatgttgtctacttgaacccggacagacaggctgtggttccttgtcgggtgaccgtgctgtcggccaaagtcacgctccacagggaattcccagccaaggagatcccagccaatggaacggacattgtttatgacatgaagcggggctttgtgtatctgcaacctcattccgagcaccagggtgtggtttactgcagggcggaggccgggggcagatctcagatctccgtcaagtaccagctgctctacgtggcggttcccagtggccctccctcaacaaccatcttggcttcttcaaacaaagtgaaaagtggggacgacatcagtgtgctctgcactgtcctgggggagcccgatgtggaggtggagttcacctggatcttcccagggcagaaggatgaaaggcctgtgacgatccaagacacttggaggttgatccacagaggactgggacacaccacgagaatctcccagagtgtcattacagtggaagactttgagacgattgatgcaggatattacatttgcactgctcagaatcttcaaggacagaccacagtagctaccactgttgagttttcctgacttggaaaaggaaatgtaatgaacttatggaaagcccatttgtgtacacagtcagctttggggttccttttattagtgctttgccagaggctgatgtcaagcaccacaccccaaccccagcgtctcgtgagtccgacccagacatccaaactaaaaggaagtcatccagtctattcacagaagtgttaacttttctaacagaaagcatgattttgattgcttacctacatacgtgttcctagtttttatacatgtgtaaacaattttatataatcaatcatttctattaaatgagcacgtttttgtaaaaaataaaaagtgggataagtgttaaaaaaaaaaaaaaaaaa

SEQ ID NO: 14 - DNA, 3677 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 15 - DNA, 3554 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 16 - DNA, 18 bp, Artificial Sequence
  Synthetic Polynucleotide
  misc_feature (1)..(6): modified with O-methyl
  misc_feature (7)..(13): modified with phosphorothioate bond
  misc_feature (13)..(18): modified with O-methyl
ugggagtagatgagguac

SEQ ID NO: 17 - DNA, 3492 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 18 - DNA, 18 bp, Artificial Sequence
  Synthetic Polynucleotide
  misc_feature (1)..(18): modified with phosphorothioate bond
  misc_feature (1)..(6): modified with O-methyl
  misc_feature (13)..(18): modified with O-methyl
ugggagtagatgagguac

SEQ ID NO: 19 - DNA, 3392 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 20 - DNA, 3488 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 21 - DNA, 3422 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 22 - DNA, 3517 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaaggagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 23 - DNA, 3554 bp, Homo sapiens; identical to SEQ ID NO: 15

SEQ ID NO: 24 - DNA, 3608 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 25 - DNA, 3624 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 26 - DNA, 3677 bp, Homo sapiens; identical to SEQ ID NO: 14

SEQ ID NO: 27 - DNA, 2569 bp, Homo sapiens
agcccgggcctggccggccgcgtgttcccggagcctcggctgcccgaatggggagcccagagtggcgagcggcacccctccccccgccagccctccgcgggaaggtgacctctcgagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaaaa

SEQ ID NO: 28 - DNA, 1822 bp, Homo sapiens
gccgtccccgccgccgctgcccgccgccaccggccgcccgcccgcccggctcctccggccgcctccgctgcgctgcgctgcgctgcctgcacccagggctcgggagggggccgcggaggagtcgccccccgcgcccggcccccgcccgccgcgcccgggcccgcgccatggggctctggctgtcgccgccccccgcgccgccgggctagggcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggagctgtccctgcctgtgtcactgtttgtcactgtccaggctggctggtttgggcatgaatgtctgcatcactaaatccagagcttgtcttgctccctcattgtgcagatggaggaaatgaggactaaggccccacagcagatcccaggcagggccagaattatgtattcatcactttcaagttattgccacgcatgggagtcagggatagcccagtcaatacagactgcctgccctcctgctcttcaccagggttcttttctagaaggagacagccttctgtggccagagagcttggggtaggacccagatctactgagtgaccttgcttgtcactacccctgcctctctgagcagcagtttccacatgtgcacatagagggaacagaagattgctgtggttggcgtcctcgggccccagagaagtttgagactatctttacgtaatagaaaagaacacttgttcttcctgccaggcaaaaaaaaaaaaaaaaaa

SEQ ID NO: 29 - DNA, 1721 bp, Homo sapiens
gccgtccccgccgccgctgcccgccgccaccggccgcccgcccgcccggctcctccggccgcctccgctgcgctgcgctgcgctgcctgcacccagggctcgggagggggccgcggaggagtcgccccccgcgcccggcccccgcccgccgcgcccgggcccgcgccatggggctctggctgtcgccgccccccgcgccgccgggctagggcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggagctgtccctgcctgtgtcactgtttgtcactgtccaggctggctggtttgggcatgaatgtctgcatcactaaatccagagcttgtcttgctccctcattgtgcagatggaggaaatgaggactaaggccccacagcagatcccaggcagggccagaattatgtattcatcactttcaagttattgccacgcatgggagtcagggatagcccagtcaatacagactgcctgccctcctgctcttcaccagggttcttttctagaaggagacagccttctgtggccagagagcttggggtaggacccagatctactgagtgaccttgcttgtcactacccctgcctctctgagcagcagtttccacatgtgcacatagagggaacagaagattgctgtggttggcgtcctcgggccccagagaagtttgagactatctttacgtaatagaaaagaacacttgttcttcctgccaggcaaaaaaaaaaaaaaaaaa

SEQ ID NO: 30 - DNA, 1719 bp, Homo sapiens
gccgtccccgccgccgctgcccgccgccaccggccgcccgcccgcccggctcctccggccgcctccgctgcgctgcgctgcgctgcctgcacccagggctcgggagggggccgcggaggagtcgccccccgcgcccggcccccgcccgccgcgcccgggcccgcgccatggggctctggctgtcgccgccccccgcgccgccgggctagggcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcaggggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggagctgtccctgcctgtgtcactgtttgtcactgtccaggctggctggtttgggcatgaatgtctgcatcactaaatccagagcttgtcttgctccctcattgtgcagatggaggaaatgaggactaaggccccacagcagatcccaggcagggccagaattatgtattcatcactttcaagttattgccacgcatgggagtcagggatagcccagtcaatacagactgcctgccctcctgctcttcaccagggttcttttctagaaggagacagccttctgtggccagagagcttggggtaggacccagatctactgagtgaccttgcttgtcactacccctgcctctctgagcagcagtttccacatgtgcacatagagggaacagaagattgctgtggttggcgtcctcgggccccagagaagtttgagactatctttacgtaatagaaaagaacacttgttcttcctgccaggcaaaaaaaaaaaaaaaaaa

SEQ ID NO: 31 - DNA, 2103 bp, Homo sapiens
acttcggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattataagctgaaaaaaaaaaaaaaaaa

SEQ ID NO: 32 - DNA, 2084 bp, Homo sapiens
aagacacatgcttctgcaagcttccatgaaggttgtgcaaaaaagtttcaatccagagttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccatataaaaaaaaaaaa

SEQ ID NO: 33 - DNA, 7123 bp, Homo sapiens
atcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacgagaggacggactctggcggccgggtcgttggccgcggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatacgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgcttttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccagggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagaactttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagaggagttttctccatatcaaaacgagggctgatggaggaaaaaggtcaataaggtcaagggaaaaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtatttatgatgtctgcaccttctgcagccagtcagaagctggagaggcaacagtggattgctgcttcttggggagaagagtatgcttccttttatccatgtaatttaactgtagaacctgagctctaagtaaccgaagaatgtatgcctctgttcttatgtgccacatccttgtttaaaggctctctgtatgaagagatgggaccgtcatcagcacattccctagtgagcctactggctcctggcagcggcttttgtggaagactcactagccagaagagaggagtgggacagtcctctccaccaagatctaaatccaaacaaaagcaggctagagccagaagagaggacaaatctttgttcttcctcttctttacatacgcaaaccacctgtgacagctggcaattttataaatcaggtaactggaaggaggttaaacacagaaaaaagaagacctcagtcaattctctacttttttttttttttccaaatcagataatagcccagcaaatagtgataacaaataaaaccttagctattcatgtcttgatttcaataattaattcttaatcattaagagaccataataaatactccttttcaagagaaaagcaaaaccattagaattgttactcagctccttcaaactcaggtttgtagcatacatgagtccatccatcagtcaaagaatggttccatctggagtcttaatgtagaaagaaaaatggagacttgtaataatgagctagttacaaagtgcttgttcattaaaatagcactgaaaattgaaacatgaattaactgataatattccaatcatttgccatttatgacaaaaatggttggcactaacaaagaacgagcacttcctttcagagtttctgagataatgtacgtggaacagtctgggtggaatggggctgaaaccatgtgcaagtctgtgtcttgtcagtccaagaagtgacaccgagatgttaattttagggacccgtgccttgtttcctagcccacaagaatgcaaacatcaaacagatactcgctagcctcatttaaattgattaaaggaggagtgcatctttggccgacagtggtgtaactgtatgtgtgtgtgtgtgtgtgtgtgtgtgtgtgtgtgtgggtgtatgtgtgttttgtgcataactatttaaggaaactggaattttaaagttacttttatacaaaccaagaatatatgctacagatataagacagacatggtttggtcctatatttctagtcatgatgaatgtattttgtataccatcttcatataataaacttccaaaaacaca

SEQ ID NO: 34 - DNA, 3632 bp, Homo sapiens
gaattccgggtgatttcactcccggctgtccaggcttgtcctgctaccccacccagcctttcctgaggcctcaagcctgccaccaagcccccagctccttctccccgcaggacccaaacacaggcctcaggactcaacacagcttttccctccaacccgttttctctccctcaacggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggcttcaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagaggtgagtgcctggccagccttcatccactctcccacccaaggggaaatgagagacgcaagagagggagagagatgggatgggtgaaagatgtgcgctgatagggagggatgagagagaaaaaaacatggagaaagacggggatgcagaaagagatgtggcaagagatggggaagagagagagagaaagatggagagacaggatgtctggcacatggaaggtgctcactaagtgtgtatggagtgaatgaatgaatgaatgaatgaacaagcagatatataaataagatatggagacagatgtggggtgtgagaagagagatgggggaagaaacaagtgatatgaataaagatggtgagacagaaagagcgggaaatatgacagctaaggagagagatgggggagataaggagagaagaagatagggtgtctggcacacagaagacactcagggaaagagctgttgaatgctggaaggtgaatacacagatgaatggagagagaaaaccagacacctcagggctaagagcgcaggccagacaggcagccagctgttcctcctttaagggtgactccctcgatgttaaccattctccttctccccaacagttccccagggacctctctctaatcagccctctggcccaggcagtcagtaagtgtctccaaacctctttcctaattctgggtttgggtttgggggtagggttagtaccggtatggaagcagtgggggaaatttaaagttttggtcttgggggaggatggatggaggtgaaagtaggggggtattttctaggaagtttaagggtctcagctttttcttttctctctcctcttcaggatcatcttctcgaaccccgagtgacaagcctgtagcccatgttgtaggtaagagctctgaggatgtgtcttggaacttggagggctaggatttggggattgaagcccggctgatggtaggcagaacttggagacaatgtgagaaggactcgctgagctcaagggaagggtggaggaacagcacaggccttagtgggatactcagaacgtcatggccaggtgggatgtgggatgacagacagagaggacaggaaccggatgtggggtgggcagagctcgagggccaggatgtggagagtgaaccgacatggccacactgactctcctctccctctctccctccctccagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacagagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaacatggtctccttcttggaattaattctgcatctgcctcttcttgtgggtgggaagaagctccctaagtcctctctccacaggctttaagatccctcggacccagtcccatccttagactcctagggccctggagaccctacataaacaaagcccaacagaatattccccatcccccaggaaacaagagcctgaacctaattacctctccctcagggcatgggaatttccaactctgggaattc

SEQ ID NO: 35 - DNA, 18 bp, Homo sapiens
gtacctcatctactccca

SEQ ID NO: 36 - DNA, 6055 bp, Homo sapiens
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 37 - DNA, 4532 bp, Homo sapiens
aggccagccggcgcccgcgcggacactttcagccccgagccgcggccgctcgggtcggacccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccagcttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaggggttaggcctccggatgactgcgggcaggcctggataatatccagcctcccacaagaagctggtggagcagagtgttccctgactcctccaaggaaagggagacgccctttcatggtctgctgagtaacaggtgccttcccagacactggcgttactgcttgaccaaagagccctcaagcggcccttatgccagcgtgacagagggctcacctcttgccttctaggtcacttctcacaatgtcccttcagcacctgaccctgtgcccaccagttattccttggtaatatgagtaatacatcaaagagtagtattaaaagctaattaatcatgtttataaaaaaaaaaaaaaaaaaaaaaaaaaaaaaa

SEQ ID NO: 38 - DNA, 2058 bp, Homo sapiens
gcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccaacagaacactaaacagactatttaacttgagggtaataaacttagaataaaattgtaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaagtctaaagtttacagtaaataaattgtttgaccatgccttcattgcaaaaaaaaaaaaaaaaa

SEQ ID NO: 39 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 40 - DNA, 5168 bp, Homo sapiens
gtggccggcggccggagccgactcggagcgcgcggcgccggccgggaggagccggagagcggccgggccgggcggtgggggcgccggcctgccccgcgcgccccagggagcggcaggaatgtgacaatcgcgcgcccgcgcaccgaagcactcctcgctcggctcctagggctctcgcctctgagctgagccgggttccgcccggggctgggatcccatcaccctccacggccgtccgtccaggtagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctgcacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaaaaaaaaaaaaaaaaa

SEQ ID NO: 41 - DNA, 1482 bp, Homo sapiens
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 42 - DNA, 2114 bp, Homo sapiens
aaagggggaaaagaaagtgcggcggaaagtaagaggctcactggggaagactgccgggatccaggtctccggggtccgctttggccagaggcgcggaaggaagcagtgcccggcgacactgcacccatcccggctgcttttgctgcgccctctcagcttcccaagaaaggcatcgtcatgtgatcatcacctaagaactagaacatcagcaggccctagaagcctcactcttgcccctccctttaatatctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaaggtaatagatgcggtcagtgatgaatctctcagctccaaattaacattgtggtgaataaggacaaaaggagagattgagaacaagagagctccagcacctagcccgacggcatctaacccatagtaatgaatcaaacttaaatgaaaaatatgaaagttttcatctatgtaagatactcaaaatattgtttctgatattgttagtaccgtaatgcccaaatgtagctaaaaaaatcgacgtgagtacagtgagacacaattttgtgtctgtacaattatgaaaaattaaaaacaaagaaaatattcaaagctaccaaagatagaaaaaactggtagagccacatattgttggtgaattattaagacccttttaaaaatcattcatggtagacttcaagagtcataaaaaagattgcatcatctgacctaagactttcggaatttttcctgaacaaataacagaaagggaattatataccttttaatattattagaagcattatctgtagttgtaaaacattattaatagcagccatccaattgtatgcaactaattaaggtattgaatgtttattttccaaaaatgcataattataatattattttaaacactatgtatcaatatttaagcaggtttataatataccagcagccacaattgctaaaatgaaaatcatttaaattatgattttaaatggtataaacatgatttctatgttgatagtactatattattctacaataaatggaaattataaagccttcttgtcagaagtgctgctcctaaaaaaaaaaaaaaaaaaaaaa

SEQ ID NO: 43 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

(19) United States
(12) Patent Application Publication
(10) Pub. No.: US 2022/0362398 A1
(43) Pub. Date: Nov. 17, 2022

(54) ADMINISTRATION OF SPHERICAL NUCLEIC ACIDS FOR OPHTHALMOLOGICAL USES

(71) Applicant: Exicure Operating Company, Chicago, IL (US)

(72) Inventors: Bart Anderson, Morton Grove, IL (US); Richard Kang, Wilmette, IL (US)

(73) Assignee: Exicure Operating Company, Chicago, IL (US)

(21) Appl. No.: 17/605,784

(22) PCT Filed: Apr. 24, 2020
(86) PCT No.: PCT/US2020/029961

§ 371 Date: Oct. 22, 2021

Related U.S. Application Data
(60) Provisional application No. 62/839,559, filed on Apr. 26, 2019.

(51) Int. Cl. A61K 47/69; A61K 9/00; A61K 31/7088; A61K 31/7084; A61K 9/127; A61P 27/02; C12N 15/11

(52) U.S. Cl. CPC A61K 47/6911; A61K 9/0048; A61K 31/7088; A61K 31/7084; A61K 9/127; A61P 27/02; C12N 15/111

(57) ABSTRACT

The present application is directed to the intravitreal use of spherical nucleic acid (SNA) molecules in the treatment of eye disorders including dry-eye, retinal edema, retinopathy, macular degeneration and glaucoma among others. SNAs have a liposomal core with a plurality of oligonucleotides on the surface forming a shell, wherein the oligonucleotide preferably a TNF-alpha antisense oligonucleotide.

### RELATED APPLICATION

This application claims priority under 35 U.S.C. § 119(e) to U.S. Provisional Application Ser. No. 62/839,559, filed on Apr. 26, 2019, which is herein incorporated by reference in its entirety.

### BACKGROUND

Spherical nucleic acids (SNAs) are a novel class of therapeutic molecules consisting of densely packed oligonucleotides arranged radially around a spherical nanoparticle core. As a consequence of their 3-dimensional structure, SNAs have an increased cellular uptake when compared with the same oligonucleotide sequence in the conventional linear format.

SNA delivery and activity have been previously demonstrated in various tissues including topical administration to the skin, aerosol delivery to the lung, oral delivery to gastrointestinal tissues, and intracerebral ventricular injection to the central nervous system, and display increased distribution and persistence as compared to linear oligonucleotides.

### SUMMARY

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting.

According to some aspects, methods of treating an eye disorder or eye disease in a subject are disclosed herein.

In some embodiments, the method comprises administering to a subject through intravitreal injection an effective amount of a spherical nucleic acid (SNA) comprising oligonucleotides forming an oligonucleotide shell to treat the eye disorder or eye disease in the subject.

According to some aspects, methods of increasing the persistence of an oligonucleotide in the eye of the subject are disclosed herein.

In some embodiments, the method comprises administering to a subject through intravitreal injection a SNA comprising oligonucleotides forming an oligonucleotide shell to increase the persistence of the oligonucleotide in the eye of the subject, wherein the persistence of the oligonucleotides in the SNA is increased relative to linear oligonucleotides that are not in a SNA.

According to some aspects, methods of delivering an oligonucleotide to one or more regions in the eye of the subject are disclosed herein.

In some embodiments, the method comprises administering to the subject through intravitreal injection a SNA comprising oligonucleotides forming an oligonucleotide shell to reach one or more regions of the eye, wherein the one or more regions of the eye comprise the posterior segment or anterior segment of the eye of the subject.

In some embodiments, the oligonucleotide is a tumor necrosis factor (TNF)-α inhibitor, a receptor tyrosine kinase (RTK) inhibitor, a cyclooxygenase (COX) inhibitor, an interleukin 1 beta (IL1β) inhibitor, a beta-2 adrenergic receptor (ADRB2) inhibitor, a connective tissue growth factor (CTGF) inhibitor or a vascular endothelial growth factor (VEGF) inhibitor, a platelet-derived growth factor subunit A (PDGFA) inhibitor, a platelet-derived growth factor subunit B (PDGFB) inhibitor, a platelet-derived growth factor subunit C (PDGFC) inhibitor, a platelet-derived growth factor subunit D (PDGFD) inhibitor, a platelet-derived growth factor receptor alpha (PDGFRA) inhibitor, a platelet-derived growth factor receptor beta (PDGFRB) inhibitor, a platelet-derived growth factor receptor like (PDGFRL) inhibitor, a vascular endothelial growth factor A (VEGFA) inhibitor, a vascular endothelial growth factor B (VEGFB) inhibitor, a vascular endothelial growth factor C (VEGFC) inhibitor, a vascular endothelial growth factor D (VEGFD) inhibitor, a vascular endothelial growth factor receptor-1 inhibitor, a vascular endothelial growth factor receptor-2 inhibitor, a vascular endothelial growth factor receptor-3 inhibitor, a beta-2 adrenergic receptor inhibitor, a connective tissue growth factor inhibitor, an interleukin-1β inhibitor, an interleukin-1 receptor-1 inhibitor, an interleukin-1 receptor-2 inhibitor, or an interleukin-1 receptor-3 inhibitor.

In some embodiments, the oligonucleotide is an antisense oligonucleotide. In some embodiments, the TNF-α inhibitor is a TNF-α antisense oligonucleotide. In some embodiments, the TNF-α antisense oligonucleotide is 8-40 nucleotides in length. In some embodiments, the oligonucleotide is not a TNF-α antisense oligonucleotide.

In some embodiments, the oligonucleotide is a DNA oligonucleotide, a DNA-RNA hybrid oligonucleotide, or an RNA oligonucleotide. In some embodiments, the RNA oligonucleotide is an siRNA, miRNA, mRNA, non-coding RNA, or aptamer.

In some embodiments, the oligonucleotides comprise a phosphorothioate modification.

In some embodiments, the SNA is administered to the subject at a dose of between 2 μg and 1 mg. In some embodiments, the SNA is administered to the subject at a dose of between 2 μg and 20 μg.

In some embodiments, the SNA is delivered to the retina or cornea of the subject.

In some embodiments, the SNA is delivered to the inner corneal surface and the outer corneal surface of the subject. In some embodiments, the SNA is delivered to the inner corneal surface or the outer corneal surface of the subject.

In some embodiments, the eye disorder or eye disease is associated with ocular angiogenesis, ocular neovascularization, retinal edema, ocular hypertension, elevated intraocular pressure, retinal ischemia, posterior segment neovascularization, age-related macular degeneration, inflammation, macular edema, uveitis, dry eye, neovascular glaucoma, glaucoma, scleritis, diabetic retinopathy, retinitis pigmentosa, optic nerve injury, retinopathy of prematurity, retinal ganglion degeneration, macular degeneration, hereditary optic neuropathy, metabolic optic neuropathy, acute ischemic optic neuropathy, commotio retinae, retinal detachment, retinal tears, retinal holes, iatrogenic retinopathy, myopia, conjunctivitis or eye cancer.

In some embodiments, the subject is a mammal. In some embodiments, the subject is human.

In some embodiments, the oligonucleotide is linked to a molecular species at the 3′ or 5′ terminus of the oligonucleotide through a linker. In some embodiments, the molecular species is linked to the oligonucleotide at the 5′ end of the oligonucleotide.

In some embodiments, the molecular species is a hydrophobic group. In some embodiments, the hydrophobic group is selected from the group consisting of cholesterol, a cholesteryl, a modified cholesteryl residue, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid, taurocholic acid, deoxycholate, oleyl litocholic acid, oleoyl cholenic acid, glycolipids, phospholipids, sphingolipids, isoprenoids, vitamins, saturated fatty acid, unsaturated fatty acid, fatty acid ester, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dye, Hoechst 33258 dye, psoralen and ibuprofen. In some embodiments, the hydrophobic group is selected from the group consisting of a steroid, vitamin E, triglyceride, Cy3 and Cy5. In some embodiments, the hydrophobic group is cholesterol.

In some embodiments, the SNA comprises a core and wherein the plurality of oligonucleotides are linked to the exterior of the core. In some embodiments, the core is a liposomal core comprising a plurality of lipids. In some embodiments, the liposomal core comprises one type of lipid. In some embodiments, the liposomal core comprises two to 10 types of lipids. In some embodiments, the lipids are selected from the group consisting of 1,2-dioleoyl-sn-glycero-3-phosphocholine (DOPC), sphingolipids, sphingosine, sphingosine phosphate, methylated sphingosines sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids, phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols, cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

In some embodiments, the lipids consist of DOPC.

In some embodiments, the oligonucleotides are indirectly linked to the core through a linker. In some embodiments, the oligonucleotides are indirectly linked to the core through more than one linker. In some embodiments, the oligonucleotides are directly linked to the core.

In some embodiments, the linker is a non-nucleotidic linker. In some embodiments, the linker is selected from the group consisting of abasic residues (dSpacer), oligoethyleneglycol, triethyleneglycol, hexaethylenegylcol, alkane-diol, or butanediol. In some embodiments, the linker is a double linker or a triple linker. In some embodiments, the double linker is two oligoethyleneglycols. In some embodiments, the linker is a hexaethyleneglycol. In some embodiments, the double linker comprises or consists of two hexaethylene glycols. In some embodiments, the two oligoethyleneglycols are hexaethylenegylcols. In some embodiments, the double linker or the triple linker is linked in between each single linker by a phosphodiester, phosphorothioate, methylphosphonate, or amide linkage.

In some embodiments, the SNA comprises two to 1,000 oligonucleotides. In some embodiments, the SNA comprises 10 to 40 oligonucleotides. In some embodiments, the SNA comprises 25 to 35 oligonucleotides. In some embodiments, the SNA comprises 30 oligonucleotides.

In some embodiments, the SNA is 10 to 40 nm in diameter. In some embodiments, the SNA is 30 nm in diameter.

In some embodiments, the liposomal core is 10 to 40 nm in diameter. In some embodiments, the liposomal core is 20 nm in diameter.

In some embodiments, the SNA is delivered in a formulation. In some embodiments, the formulation comprises 5% dextrose.

In some embodiments, the oligonucleotide is linked to the molecular species at the 3′ or 5′ terminus of the oligonucleotide through a linker, wherein the molecular species is adsorbed to the liposomal core of the SNA and the oligonucleotides forming the oligonucleotide shell extend radially from the core, and wherein the oligonucleotides forming the oligonucleotide shell comprise the entire SNA such that no other structural components are part of the SNA.

In some embodiments, the antisense oligonucleotide inhibits the expression of a gene in the eye of the subject, wherein the expression of the gene is inhibited by between 50% and 99% relative to a baseline level of expression of the gene in the eye of the subject.

In some embodiments, the antisense oligonucleotide inhibits the expression of a gene in the eye of the subject, wherein the expression of the gene is inhibited by between 50% and 99% relative to the level of inhibition of the gene in the eye of the subject with the corresponding linear antisense oligonucleotide that is not in a SNA.

In some embodiments, the antisense oligonucleotide comprises a modified nucleoside. In some embodiments, the modified nucleoside comprises a modified sugar moiety. In some embodiments, the modified sugar moiety comprises a 2′-substituent. In some embodiments, the 2′-substituent is selected from the group consisting of: 2′-O-methyl (2′-OMe), 2′-fluoro (2′-F), and 2′-O-methoxy-ethyl (2′-MOE). In some embodiments, the 2′-substituent is 2′-MOE. In some embodiments, the modified sugar moiety is a bicyclic sugar moiety. In some embodiments, the bicyclic sugar moiety is locked nucleic acid (LNA) or constrained ethyl nucleoside (cEt). In some embodiments, the modified sugar moiety comprises a sugar surrogate. In some embodiments, the sugar surrogate is a morpholino or a peptide nucleic acid (PNA).

In some embodiments, the antisense oligonucleotide comprises a backbone that comprises a phosphorothioate internucleoside linkage. In some embodiments, the phosphorothioate internucleoside linkage has a (Sp) stereochemical configuration. In some embodiments, the phosphorothioate internucleoside linkage has a (Rp) stereochemical configuration. In some embodiments, the antisense oligonucleotide comprises a backbone that consists of phosphorothioate internucleoside linkages. In some embodiments, the phosphorothioate internucleoside linkages have a (Sp) stereochemical configurations. In some embodiments, the phosphorothioate internucleoside linkages have a (Rp) stereochemical configurations. In some embodiments, the phosphorothioate internucleoside linkages have the same stereochemical configuration. In some embodiments, the phosphorothioate internucleoside linkages have different stereochemical configurations. In some embodiments, two to 30 of the phosphorothioate internucleoside linkages have the same stereochemical configuration.

In some embodiments, the antisense oligonucleotide comprises two to 30 phosphorothioate internucleoside linkages. In some embodiments, the antisense oligonucleotide consists of two to 30 phosphorothioate internucleoside linkages.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic of the SNA structure which shows the oligonucleotides arranged radially around a spherical nanoparticle core.

FIG. 2A is a schematic showing the timeline for eye collection. FIG. 2B is a table showing the dose amounts of Cy5-labeled oligonucleotides delivered in SNA or linear format, as well as the euthanasia time points following intravitreal injection (IVI) for the various treatment groups.

FIGS. 3A-3C show that the SNA format increases retinal distribution and persistence. (FIG. 3A) Representative images following IVI of high-dose (2.5 nmol) oligonucleotide. Arrows indicate intracellular signal, located in ganglion cells. (FIG. 3B) Semi-quantitative assessment of oligonucleotide amount and retinal surface coverage. (FIG. 3C) SNA distribution through retinal layers 24 hr following IVI of high-dose (2.5 nmol).

FIGS. 4A-4C show that the SNA format increases corneal distribution and persistence. (FIG. 4A) Representative images 24 hours following IVI of high-dose (2.5 nmol) oligonucleotide. Arrows indicate intracellular signal. (FIG. 4B) Semi-quantitative assessment of oligonucleotide amount and inner corneal surface coverage. (FIG. 4C) Semi-quantitative assessment of oligonucleotide amount and outer corneal surface coverage.

FIG. 5 shows that the intracellular SNA signal increases over time. Representative images following IVI of high-dose (2.5 nmol) oligonucleotide in SNA format. Arrows indicate intracellular signal.

FIG. 6 shows the SNA signal persists over time. Semi-quantitative assessment of oligonucleotide amount and tissue surface coverage 24 hr following IVI of high dose (2.5 nmol) oligonucleotide in SNA and linear formats.

FIG. 7 is a set of exemplary sequences for target genes.

### DETAILED DESCRIPTION

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

Disclosed herein is the biodistribution of spherical nucleic acids (SNAs) in the eye after administration, through, for instance, intravitreal injection. Surprisingly, it was determined that SNAs administered to the eye reach both posterior and anterior ocular structures, exhibit higher, wider or broader distribution than linear oligonucleotides, persist in the eye longer than linear oligonucleotides, and do not promote inflammation in the eye. These characteristics are quite advantageous because the eye has many unique barriers to drug delivery and the present disclosure addresses these challenges.

According to some aspects, methods of treating an eye disorder or eye disease in a subject are disclosed herein.

In some embodiments, the method comprises administering to a subject through intravitreal injection an effective amount of a SNA comprising oligonucleotides forming an oligonucleotide shell to treat the disease or disorder of the eye in the subject.

In some embodiments, the SNA comprises two to 1,000 oligonucleotides. In some embodiments, the SNA comprises 10 to 40 oligonucleotides. In some embodiments, the SNA comprises 25 to 35 or about 25 to 35 oligonucleotides. In some embodiments, the SNA comprises 30 or about 30 oligonucleotides. In some embodiments, the SNA comprises 5 or at least 5, 10 or at least 10, 15 or at least 15, 20 or at least 20, 21 or at least 21, 22 or at least 22, 23 or at least 23, 24 or at least 24, 25 or at least 25, 26 or at least 26, 27 or at least 27, 28 or at least 28, 29 or at least 29, 30 or at least 30, 31 or at least 31, 32 or at least 32, 33 or at least 33, 34 or at least 34, 35 or at least 35, 36 or at least 36, 37 or at least 37, 38 or at least 38, 39 or at least 39, 40 or at least 40, 41 or at least 41, 42 or at least 42, 43 or at least 43, 44 or at least 44, 45 or at least 45, 46 or at least 46, 47 argues 47, 48 or at least 48, 49 or at least 49, 50 or at least 50, 60 or at least 60, 70 or at least 70, 80 or at least 80, 90 or at least 90, 100 or at least 100, 150 or at least 150, 200 or at least 200, 250 or at least 250, 300 or at least 300, 350 or at least 350, 400 or at least 400, 450 or at least 450, 500 or at least 500, 600 or at least 600, 700 or at least 700, 800 or at least 800, 900 or at least 900, 1,000 or at least 1,000, 2,000 or at least 2,000, 3,000 or at least 3,000, 4,000 or at least 4,000, 5,000 or at least 5,000, 6,000 or at least 6,000, 7,000 or at least 7,000, 8,000 or at least 8,000, 9,000 or at least 9,000, 10,000 or at least 10,000 oligonucleotides, or any range or combination thereof.

In some embodiments, an oligonucleotide disclosed herein is positioned on the exterior surface of a core of a SNA disclosed herein. In some embodiments, 5 or at least 5, 10 or at least 10, 15 or at least 15, 20 or at least 20, 21 or at least 21, 22 or at least 22, 23 or at least 23, 24 or at least 24, 25 or at least 25, 26 or at least 26, 27 or at least 27, 28 or at least 28, 29 or at least 29, 30 or at least 30, 31 or at least 31, 32 or at least 32, 33 or at least 33, 34 or at least 34, 35 or at least 35, 36 or at least 36, 37 or at least 37, 38 or at least 38, 39 or at least 39, 40 or at least 40, 41 or at least 41, 42 or at least 42, 43 or at least 43, 44 or at least 44, 45 or at least 45, 46 or at least 46, 47 argues 47, 48 or at least 48, 49 or at least 49, 50 or at least 50, 60 or at least 60, 70 or at least 70, 80 or at least 80, 90 or at least 90, 100 or at least 100, 150 or at least 150, 200 or at least 200, 250 or at least 250, 300 or at least 300, 350 or at least 350, 400 or at least 400, 450 or at least 450, 500 or at least 500, 600 or at least 600, 700 or at least 700, 800 or at least 800, 900 or at least 900, 1,000 or at least 1,000, 2,000 or at least 2,000, 3,000 or at least 3,000, 4,000 or at least 4,000, 5,000 or at least 5,000, 6,000 or at least 6,000, 7,000 or at least 7,000, 8,000 or at least 8,000, 9,000 or at least 9,000, 10,000 or at least 10,000 oligonucleotides, or any range or combination thereof are on the exterior surface of the core of a SNA disclosed herein, such as a liposome core. In some embodiments, 30 or about 30 oligonucleotides are on the exterior surface of the core (e.g., a liposome core) of a SNA disclosed herein.

According to some aspects, methods of increasing the persistence of an oligonucleotide in the eye of the subject are disclosed herein. In some embodiments, the method comprises administering to a subject through intravitreal injection a SNA comprising oligonucleotides forming an oligonucleotide shell to increase the persistence of the oligonucleotide in the eye of the subject, wherein the persistence of the oligonucleotides in the SNA is increased relative to linear oligonucleotides that are not in a SNA.

In some embodiments, the persistence of the oligonucleotides in the SNA is increased relative to linear oligonucleotides that are not in a SNA by about 2% to about 500%, about 2% to about 450%, about 2% to about 400%, about 2% to about 350%, about 2% to about 300%, about 2% to about 250%, about 2% to about 200%, about 2% to about 175%, about 2% to about 160%, about 2% to about 150%, about 2% to about 140%, about 2% to about 130%, about 2% to about 120%, about 2% to about 110%, about 2% to about 100%, about 2% to about 95%, about 2% to about 90% about 2% to about 85% to about 2% to about 80%, about 2% to about 75%, about 2% to about 70%, about 2% to about 65%, about 2% to about 60%, about 2% to about 55%, about 2% to about 50%, about 2% to about 45% to about 2% to about 40%, about 2% to about 35%, about 2% to about 30%, about 2% to about 25%, about 2% to about 20%, about 2% to about 15%, about 2% to about 10%, about 2% to about 5%, about 10% to about 500%, about 10% to about 450%, about 10% to about 400%, about 10% to about 350%, about 10% to about 300%, about 10% to about 250%, about 10% to about 200%, about 10% to about 175%, about 10% to about 160%, about 10% to about 150%, about 10% to about 140%, about 10% to about 130%, about 10% to about 120%, about 10% to about 110%, about 10% to about 100%, about 10% to about 95%, about 10% to about 90% about 10% to about 85% to about 10% to about 80%, about 10% to about 75%, about 10% to about 70%, about 10% to about 65%, about 10% to about 60%, about 10% to about 55%, about 10% to about 50%, about 10% to about 45% to about 10% to about 40%, about 10% to about 35%, about 10% to about 30%, about 10% to about 25%, about 10% to about 20%, about 10% to about 15%, about 10% to about 10%, about 10% to about 5% more oligonucleotides in the eye of the subject relative to linear oligonucleotides that are not in a SNA.

In some embodiments, a SNA disclosed herein exhibits higher, wider and/or broader distribution in a region of the eye, a part of the eye, or on a surface of the eye relative to a linear oligonucleotide that is not in a SNA. In some embodiments, a SNA disclosed herein exhibits higher, wider and/or broader distribution by covering 10%, 15%, 20%, 25%, 30%, 35%, 40%, 45%, 50%, 55%, 60%, 65%, 70%, 75%, 80%, 85%, 90%, 95%, or 99% more of a region of the eye, a part of the eye, or a surface of the eye relative to a linear oligonucleotide that is not in a SNA. In some embodiments, a SNA disclosed herein exhibits higher, wider and/or broader distribution by covering 1.5-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, or greater than 100-fold more of a region of the eye, a part of the eye, or a surface of the eye relative to a linear oligonucleotide that is not in a SNA. The distribution of a SNA disclosed herein can be readily measured by methods known and available to one of ordinary skill in the art without undue experimentation and are also contemplated herein.

Such methods are also contemplated for any of the methods disclosed herein. Non-limiting examples include fluorescence microscopy and image analysis and/or quantification, hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell sorting (FACS) or flow cytometric analysis.

In some embodiments, a SNA disclosed herein covers 100% or about 100% of a region of the eye, a part of the eye, or a surface of the eye (e.g., retina or retinal surface, cornea, corneal surface, inner corneal surface or outer corneal surface). In some embodiments, the SNA disclosed herein covers 10% or about 10%, 15% or about 15%, 20% or about 20%, 25% or about 25%, 30% or about 30%, 35% or about 35%, 40% or about 40%, 45% or about 45%, 50% or about 50%, 55% or about 55%, 60% or about 60%, 65% or about 65%, 70% or about 70%, 75% or about 75%, 80% or about 80%, 85% or about 85%, 90% or about 90% 95% or about 95%, 99% or about 99% of a region of the eye, a part of the eye, or a surface of the eye.

In some embodiments, the persistence of the oligonucleotides in the SNA is increased relative to linear oligonucleotides that are not in a SNA when at least 10%, 15%, 20%, 25%, 30%, 35%, 40%, 45%, 50%, 55%, 60%, 65%, 70%, 75%, 80%, 85%, 90%, 95%, or 100% of the oligonucleotides in the SNA disclosed herein remain in the eye of the subject for 30 minutes longer, one hour longer longer, two hours longer, three hours longer, four hours longer, five hours longer, six hours longer, seven hours longer, eight hours longer, nine hours longer, 10 hours longer, 18 hours longer, 24 hours longer, 48 hours longer, 72 hours longer, four days longer, five days longer, six days longer, seven days longer, eight days longer, nine days longer, 10 days longer, 12 days longer, 14 days longer, 60 days longer, 18 days longer, 20 days longer, 22 days longer, 24 days longer, 26 days longer, 28 days longer, 30 days longer, 1.5 months longer, 2 months longer, 2.5 months longer, 3 months longer, 3.5 months longer, 4 months longer, 4.5 months longer, 5 months longer, 5.5 months longer, 6 months longer, 6.5 months longer, 7 months longer, 7.5 months longer, 8 months longer, 8.5 months longer, 9 months longer, 9.5 months longer, 10 months longer, 10.5 months longer, 11 months longer, 11.5 months longer, 1 year longer, 1.5 years longer, 2 years longer, 2.5 years longer, 3 years longer, 3.5 years longer, 4 years longer, 4.5 years longer, 5 years longer, 6 years longer, 7 years longer, 8 years longer, 9 years longer, 10 years longer or more than 10 years longer relative to linear oligonucleotides that are not in a SNA.

In some embodiments, the method of treating an eye disorder or eye disease in a subject comprises administering to a subject a SNA disclosed herein. In some embodiments, the SNA is delivered to reach one or more regions of the eye. In some embodiments, the region of the eye is the anterior segment or an anterior ocular structure. In some embodiments, the region of the eye is the posterior segment or a posterior ocular structure. The anterior segment of the eye occupies approximately one-third of its volume while the remaining portion of the eye is occupied by the posterior segment. Tissues such as cornea, conjunctiva, aqueous humor, iris, ciliary body and lens make up the anterior portion. The back of the eye or posterior segment of the eye includes the sclera, choroid, retinal pigment epithelium, neural retina, optic nerve and vitreous humor.

In some embodiments, the region of the eye is the retina, a region of the retinal layer, macula, choroid, sclera, fovea, physiologic cup, aqueous humor, vitreous humor, vitreous body, uvea, cornea, pupil tear film (e.g., oil layer, aqueous layer, mucin layer), keratinocyte, epithelium, stroma, endothelial layer, Bowman's membrane, Descemet's membrane, ciliary body, iris, zonules, nucleus, capsule, lens fibers, equator, lens, inner limiting membrane, nerve fiber layer, ganglion cell layer, inner plexiform layer, inner nuclear layer, outer plexiform layer, receptor nuclear layer, external limiting membrane, photoreceptors, choroid, retinal pigment epithelium, cribriform plate, central retinal artery, retinal vein, optic disc, or the optic nerve. In some embodiments, the SNA is delivered to the retina or cornea or the retina and cornea of the subject. In some embodiments, the SNA is delivered to the ciliary body. In some embodiments, the SNA is delivered to the iris. In some embodiments, the SNA is delivered to the sclera. In some embodiments, the SNA is delivered to the vitreous humor. In some embodiments, the SNA is delivered to at least one of the ciliary body, iris, sclera, and vitreous humor.

In some embodiments, the oligonucleotide is a tumor necrosis factor (TNF)-α inhibitor, a receptor tyrosine kinase (RTK) inhibitor, a cyclooxygenase (COX) inhibitor, an interleukin 1 beta (IL1β) inhibitor, a beta-2 adrenergic receptor (ADRB2) inhibitor, a connective tissue growth factor (CTGF) inhibitor or a vascular endothelial growth factor (VEGF) inhibitor, a platelet-derived growth factor subunit A (PDGFA) inhibitor, a platelet-derived growth factor subunit B (PDGFB) inhibitor, a platelet-derived growth factor subunit C (PDGFC) inhibitor, a platelet-derived growth factor subunit D (PDGFD) inhibitor, a platelet-derived growth factor receptor alpha (PDGFRA) inhibitor, a platelet-derived growth factor receptor beta (PDGFRB) inhibitor, a platelet-derived growth factor receptor like (PDGFRL) inhibitor, a vascular endothelial growth factor A (VEGFA) inhibitor, a vascular endothelial growth factor B (VEGFB) inhibitor, a vascular endothelial growth factor C (VEGFC) inhibitor, a vascular endothelial growth factor D (VEGFD) inhibitor, a vascular endothelial growth factor receptor-1 inhibitor, a vascular endothelial growth factor receptor-2 inhibitor, a vascular endothelial growth factor receptor-3 inhibitor, a beta-2 adrenergic receptor inhibitor, a connective tissue growth factor inhibitor, an interleukin-1β inhibitor, an interleukin-1 receptor-1 inhibitor, an interleukin-1 receptor-2 inhibitor, or an interleukin-1 receptor-3 inhibitor.

In some embodiments, the oligonucleotide is an antisense oligonucleotide. In some embodiments, the TNF-α inhibitor is a TNF-α antisense oligonucleotide. In some embodiments, the TNF-α antisense oligonucleotide is 8-40 nucleotides in length. In some embodiments, the oligonucleotide is not a TNF-α antisense oligonucleotide.

In some embodiments, a SNA comprises a first oligonucleotide, such as a first antisense oligonucleotide, described herein that is co-administered with one or more oligonucleotide(s) (e.g., a second oligonucleotide, a third oligonucleotide, a fourth oligonucleotide, etc.), such as a second antisense oligonucleotide. In some embodiments, the second oligonucleotide is designed to treat the same eye disease or eye disorder as the first oligonucleotide. In some embodiments, the first oligonucleotide (e.g., first antisense oligonucleotide) and the second oligonucleotide (e.g., second antisense oligonucleotide) are in the same SNA. In some embodiments, the first oligonucleotide is more abundant in the SNA than the second oligonucleotide. In some embodiments, the second oligonucleotide is more abundant in the SNA than the first oligonucleotide. In some embodiments, the SNA contains about the same amounts of the first oligonucleotide and the second oligonucleotide.

Antisense activities of an antisense oligonucleotide disclosed herein may be observed directly or indirectly. In some embodiments, observation or detection of antisense activity involves observation or detection of a change in an amount of a target nucleic acid or a protein encoded by such a target nucleic acid, a change in an amount of a protein, and/or a phenotypic change in a cell or tissue or organ.

Identifying an antisense oligonucleotide that targets a particular nucleic acid may be a multistep process. The process usually begins with the identification of a nucleic acid sequence whose function is to be modulated. This may be, for example, a cellular gene (or mRNA transcribed from the gene) whose expression is associated with a particular disorder or disease state, such as a TNF-α-associated disorder. The targeting process also includes determination of a site or sites within, for instance, this TNF-α gene or gene product (e.g., mRNA) for the antisense interaction to occur such that the desired effect, e.g., detection or modulation of expression of the protein, will result. In some embodiments, an intragenic site for the TNF-α gene is the region encompassing the nucleotide sequence 2283-2300 of SEQ ID NO: 34, i.e., gtacctca tctactccca (SEQ ID NO: 35).

In some embodiments, preferred antisense oligonucleotides are designed to target human TNF-α, platelet-derived growth factor subunit A (PDGFA), platelet-derived growth factor subunit B (PDGFB), platelet-derived growth factor subunit C (PDGFC), platelet-derived growth factor subunit D (PDGFD), platelet-derived growth factor receptor alpha (PDGFRA), platelet-derived growth factor receptor beta (PDGFRB), platelet-derived growth factor receptor like (PDGFRL), pascular endothelial growth factor A (VEGFA), vascular endothelial growth factor B (VEGFB), vascular endothelial growth factor C (VEGFC), vascular endothelial growth factor D (VEGFD), vascular endothelial growth factor receptor-1 (VEGFR1), vascular endothelial growth factor receptor-2 (VEGFR2), vascular endothelial growth factor receptor-3 (VEGFR3), beta-2 adrenergic receptor, connective tissue growth factor (CTGF), interleukin 1 beta (ILβ); interleukin 1 receptor-1 (IL1R1), interleukin 1 receptor-2 (IL1R2), and interleukin 1 receptor-3 (IL1R3).

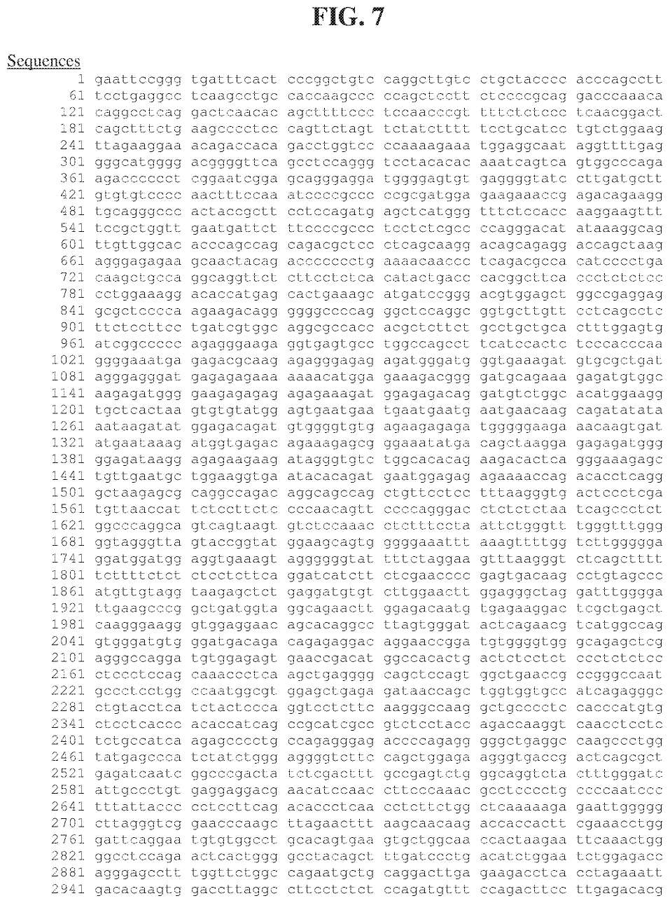

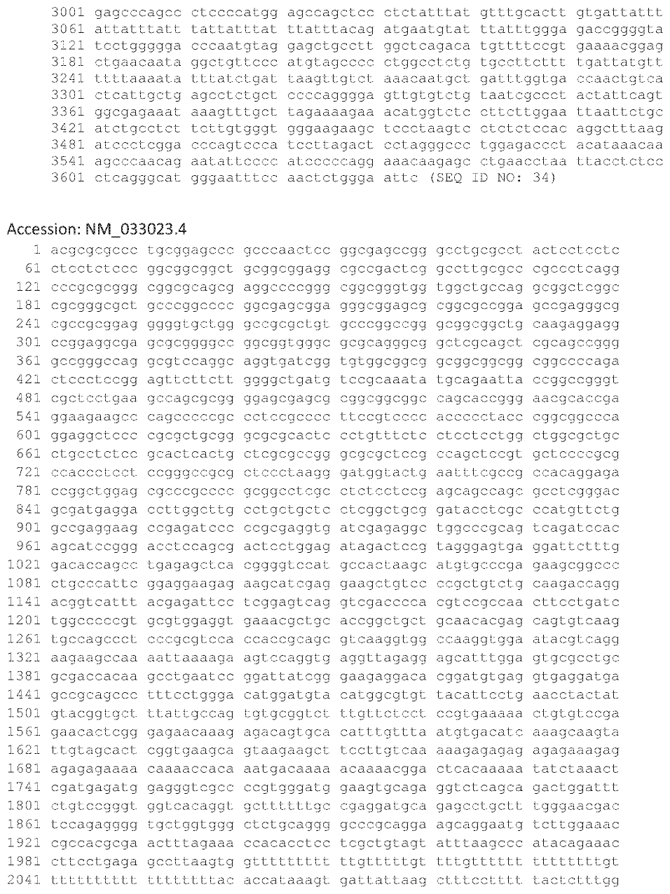

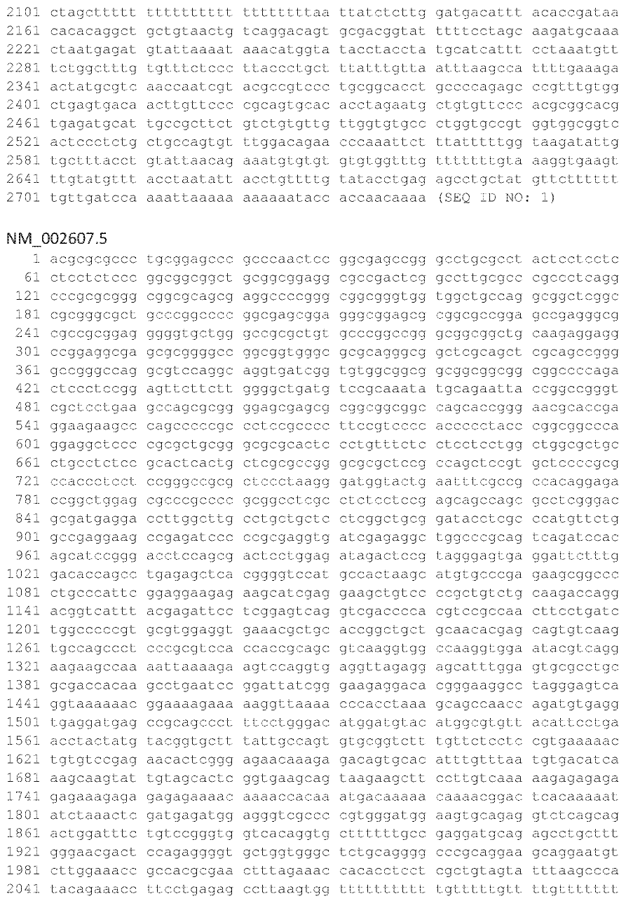

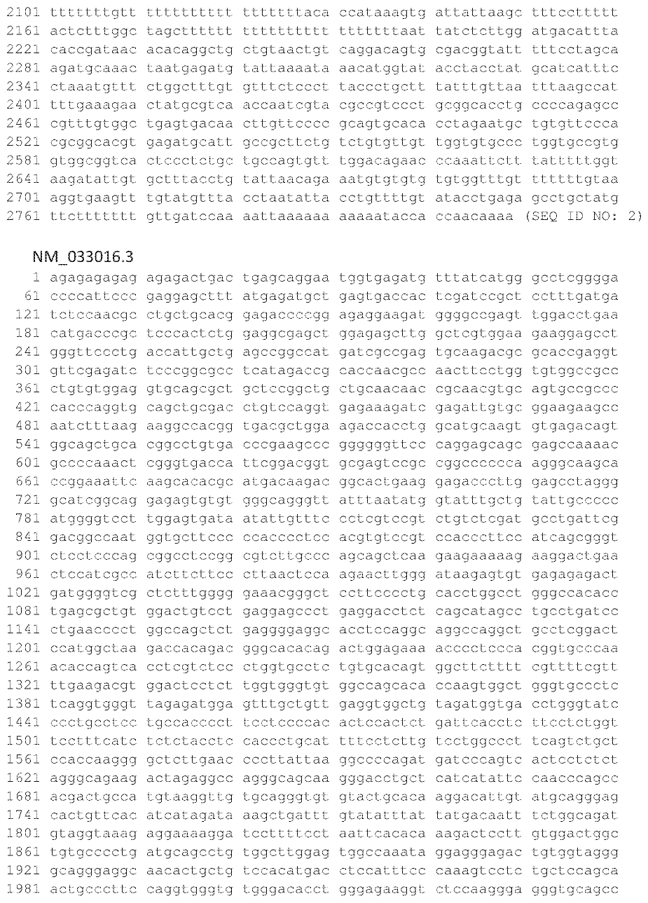

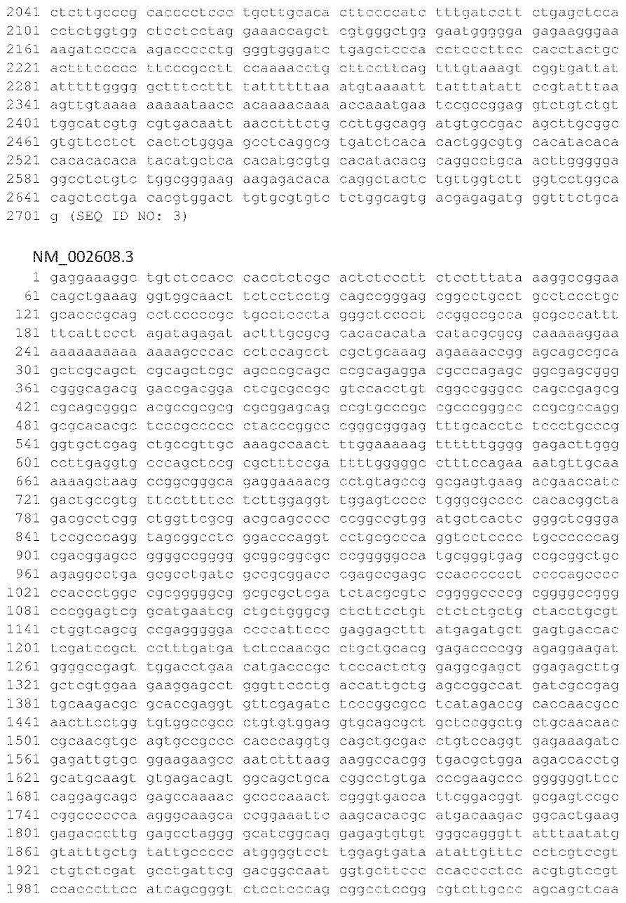

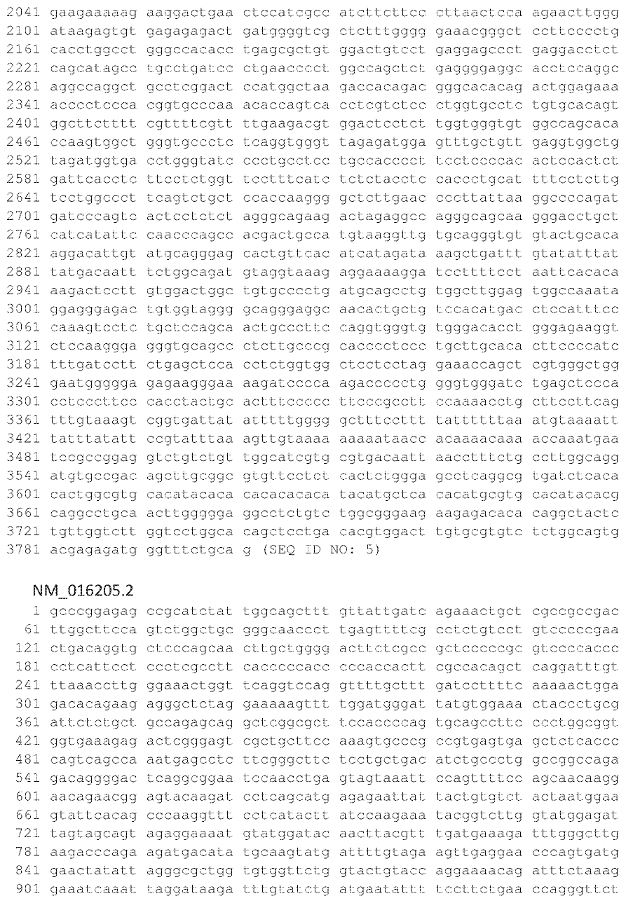

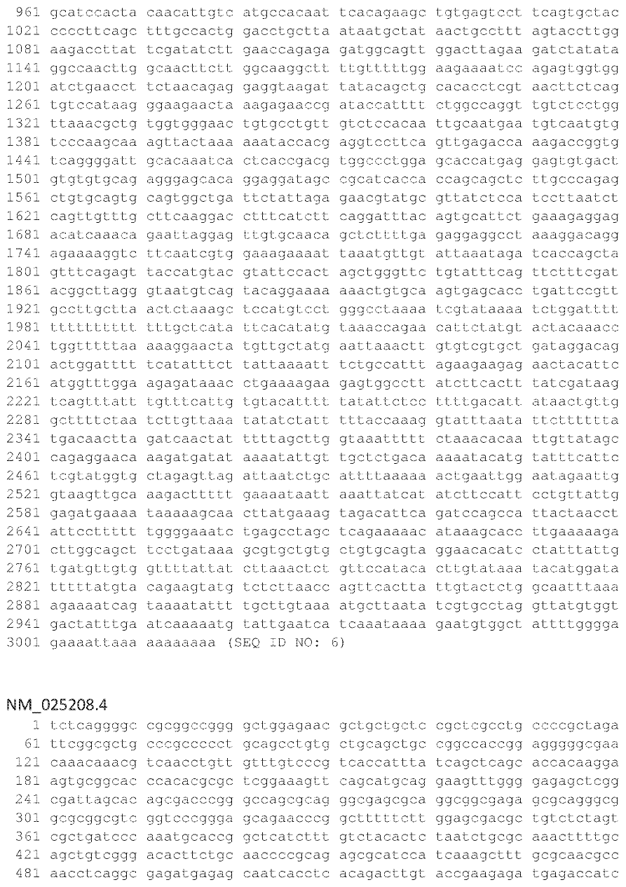

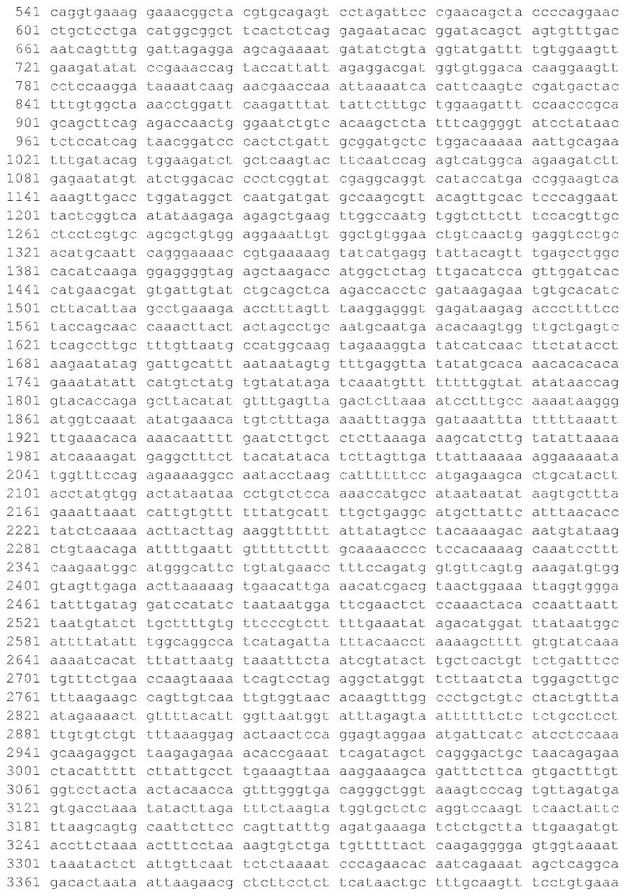

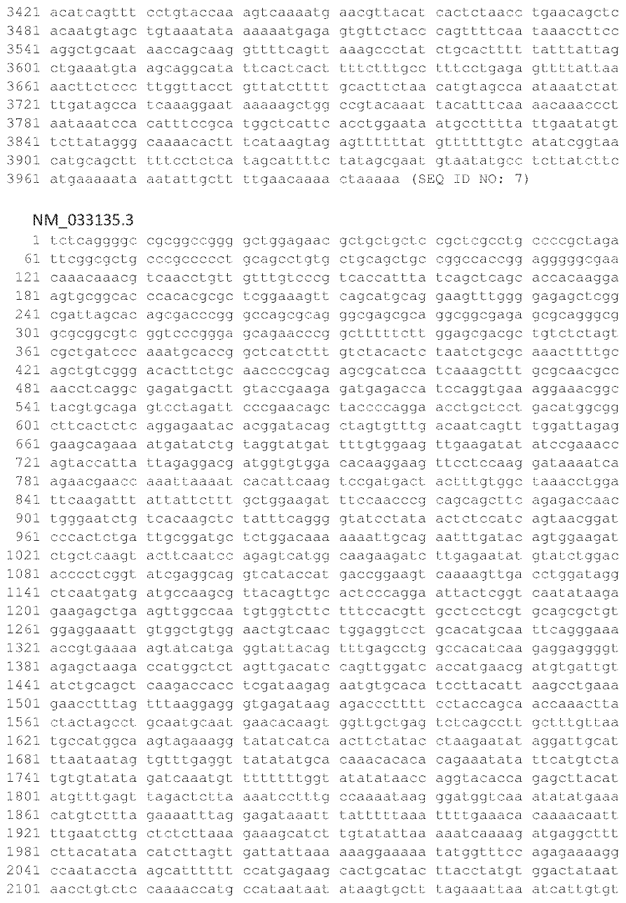

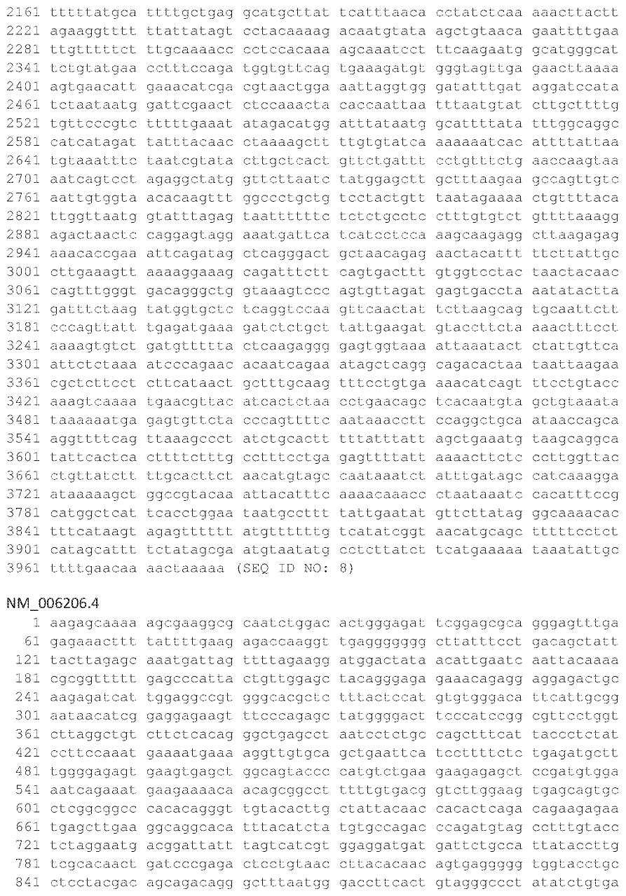

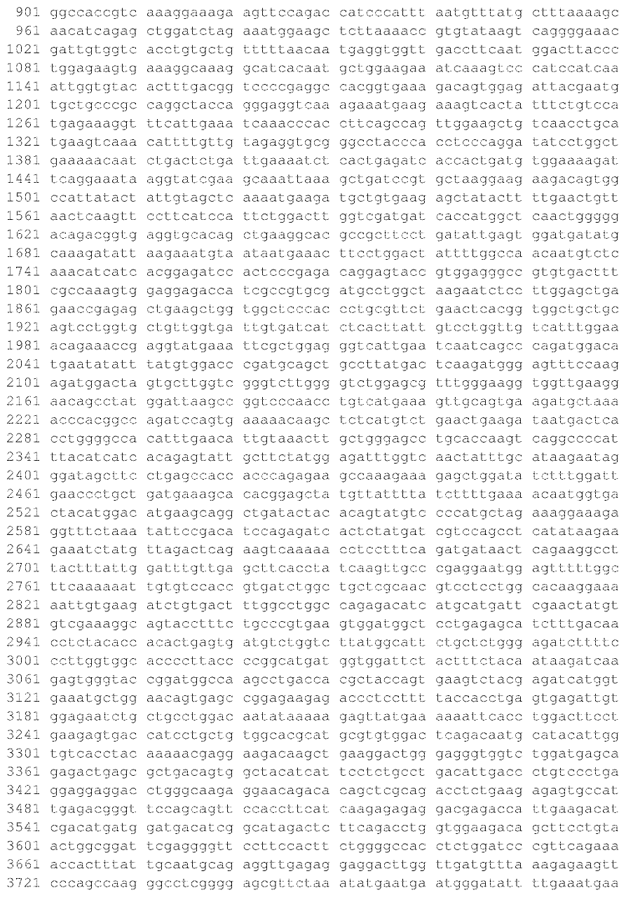

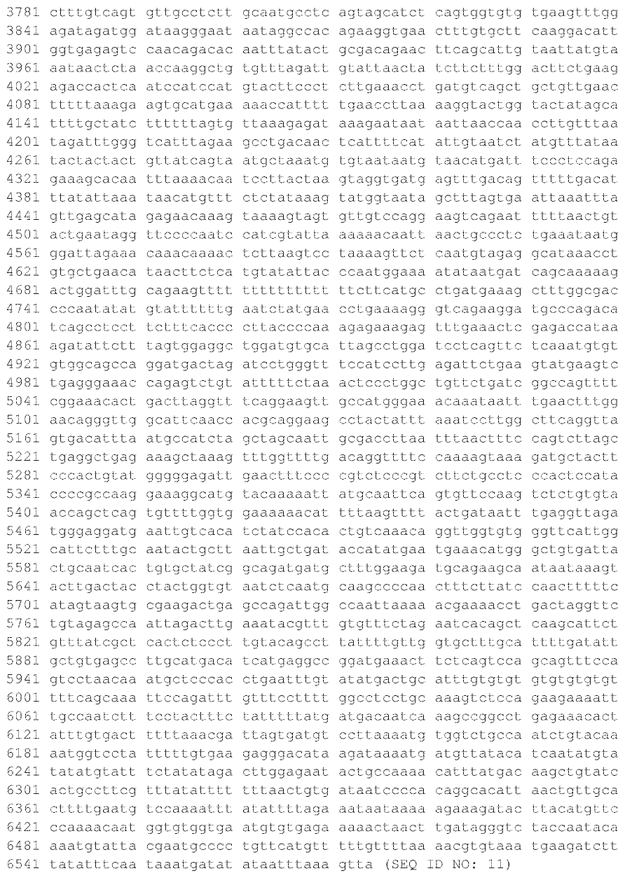

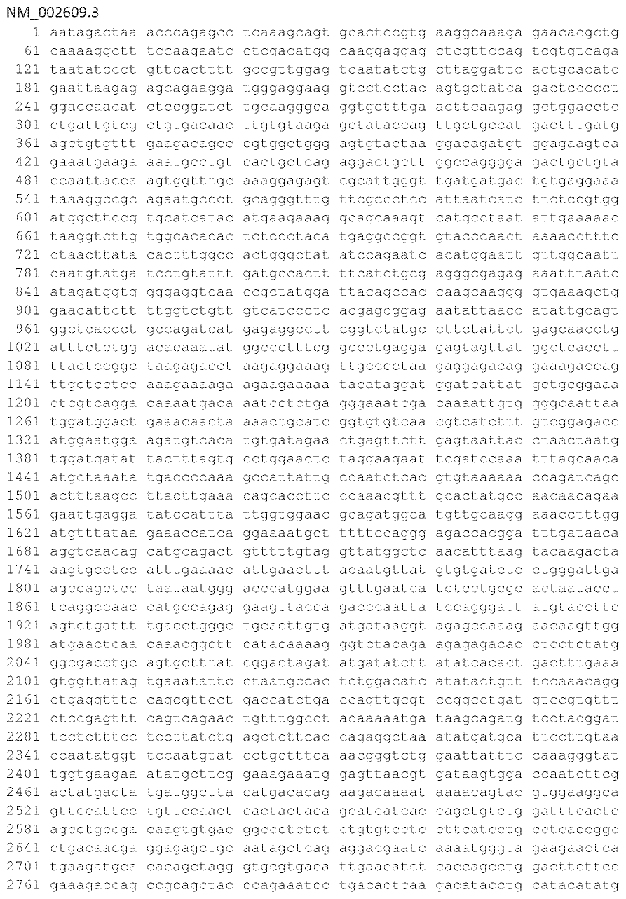

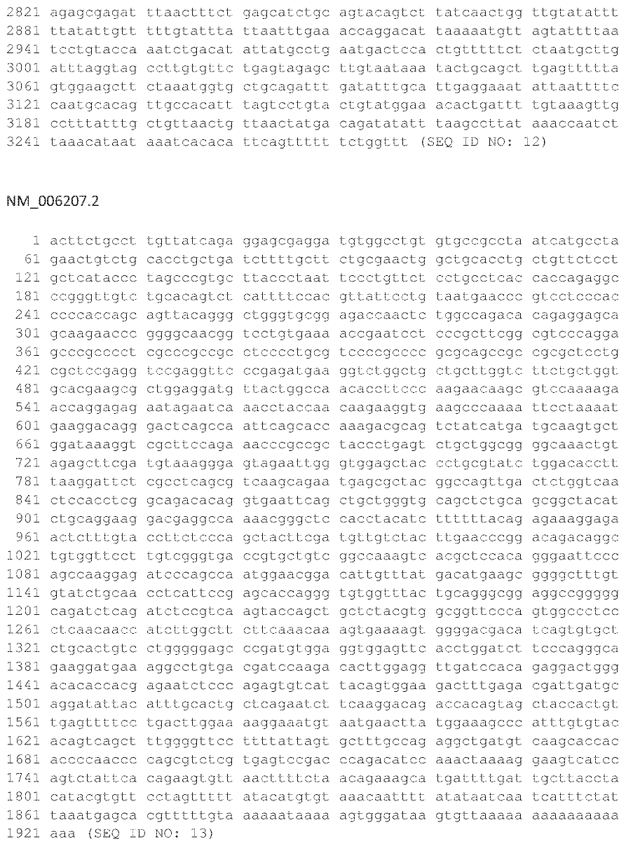

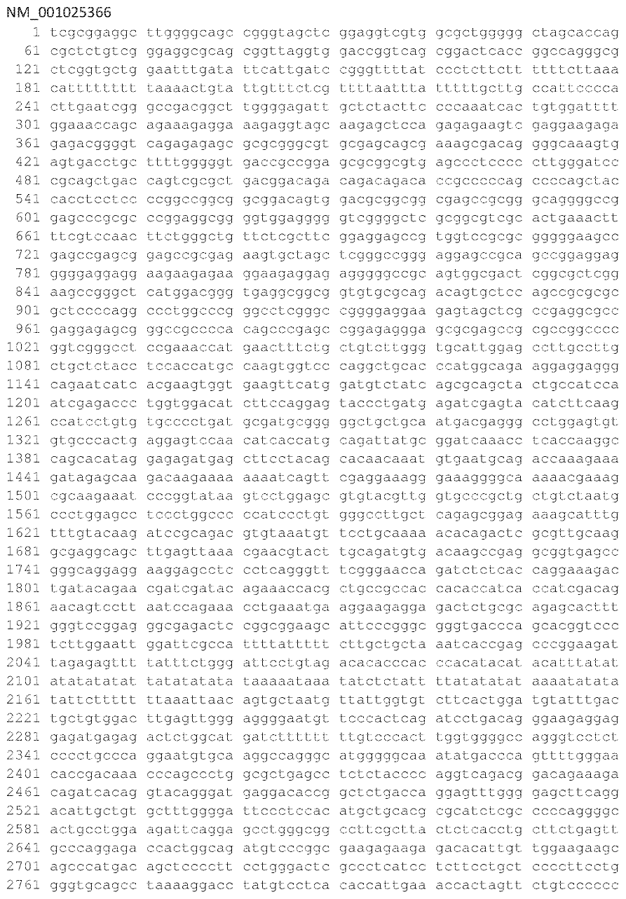

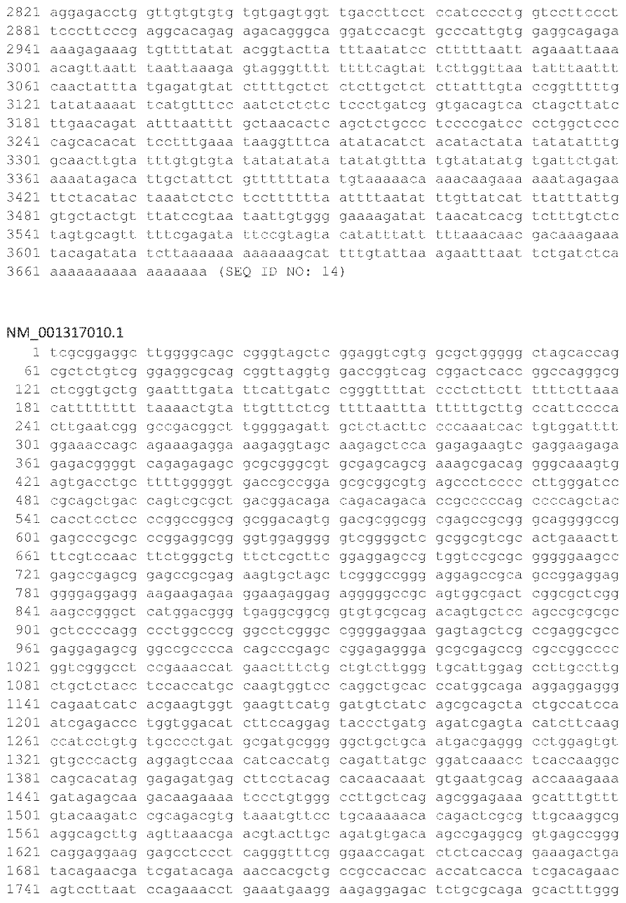

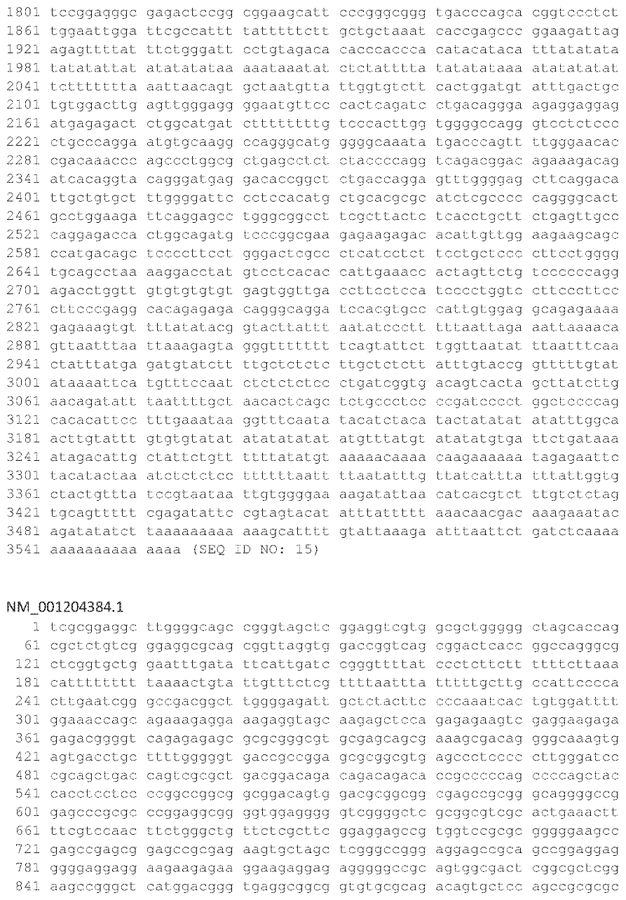

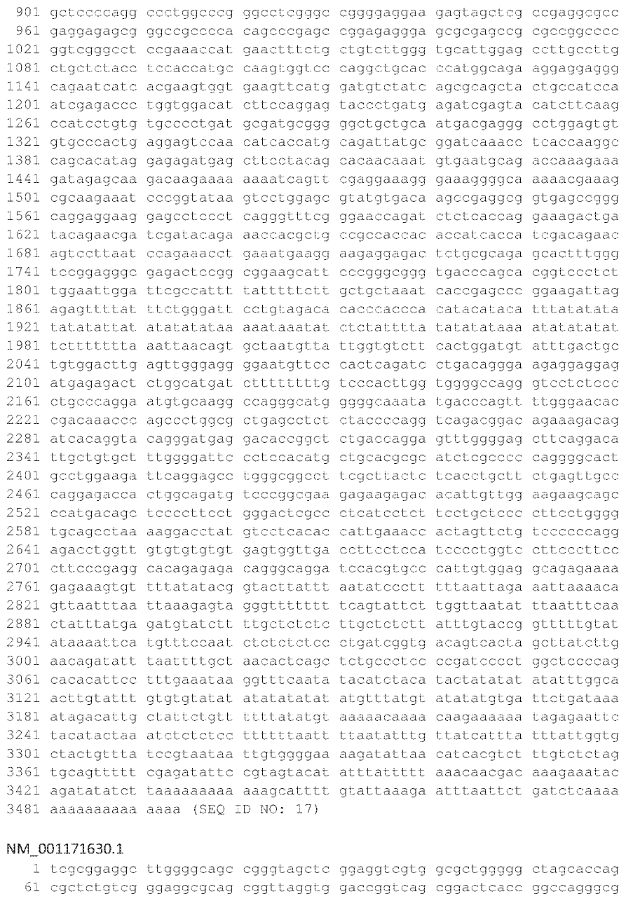

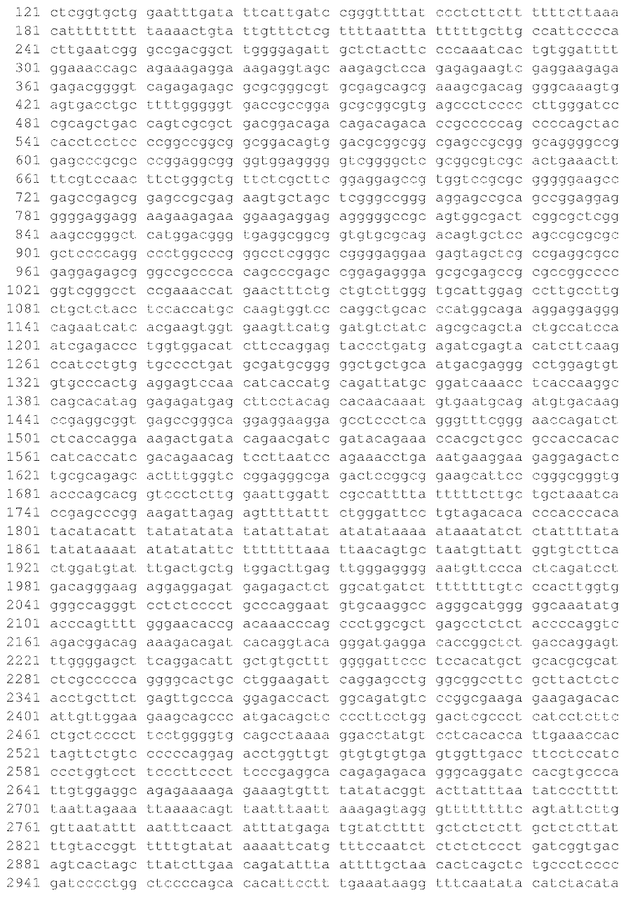

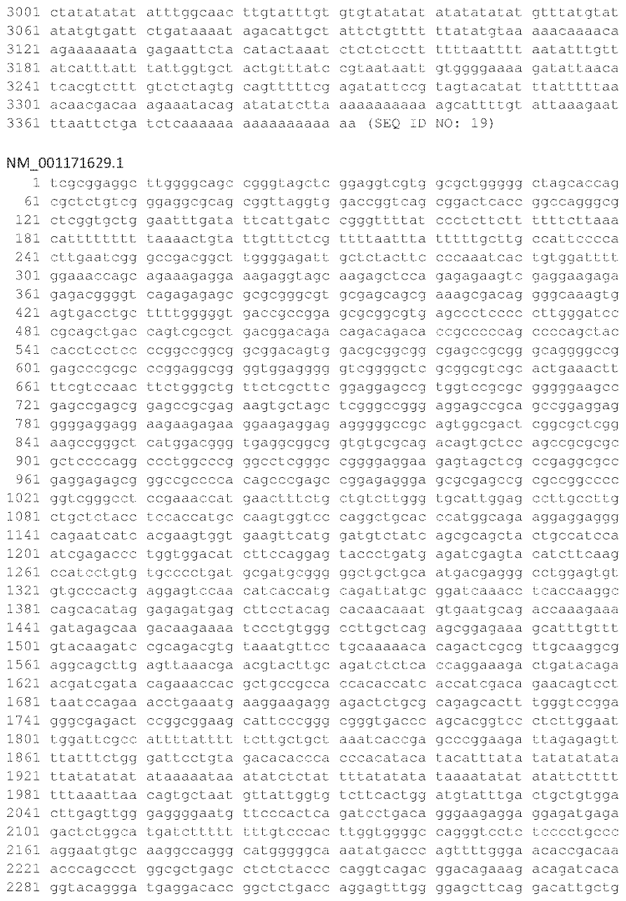

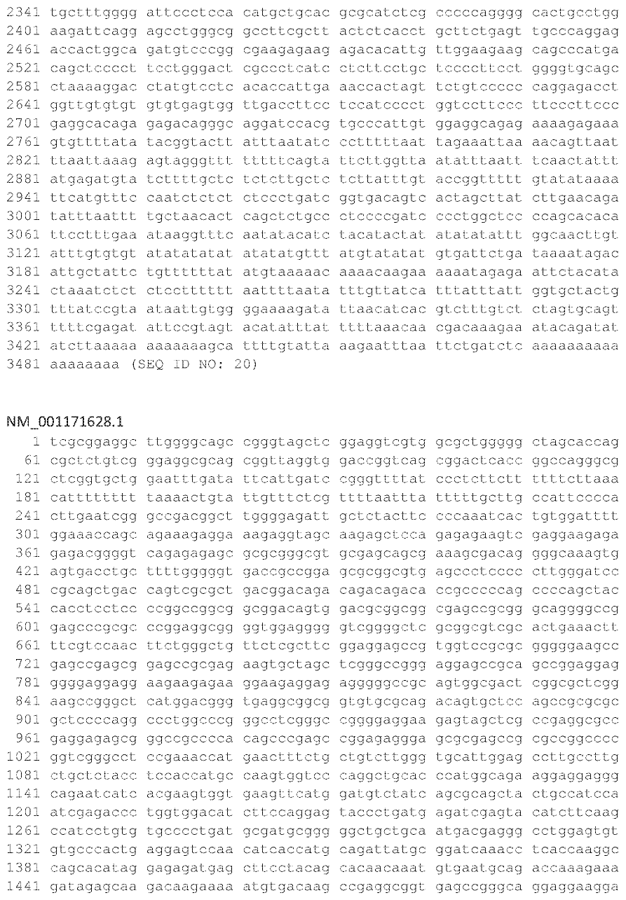

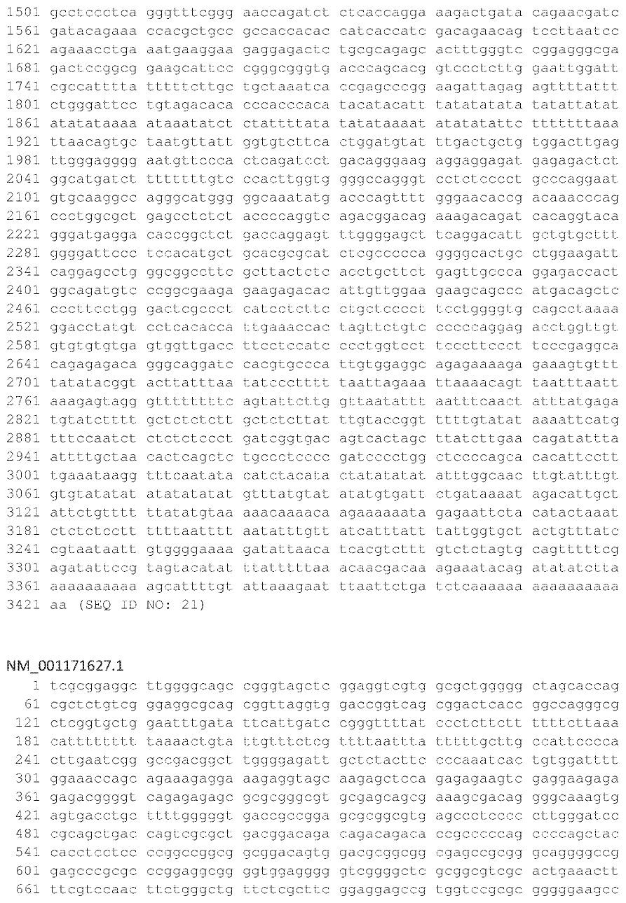

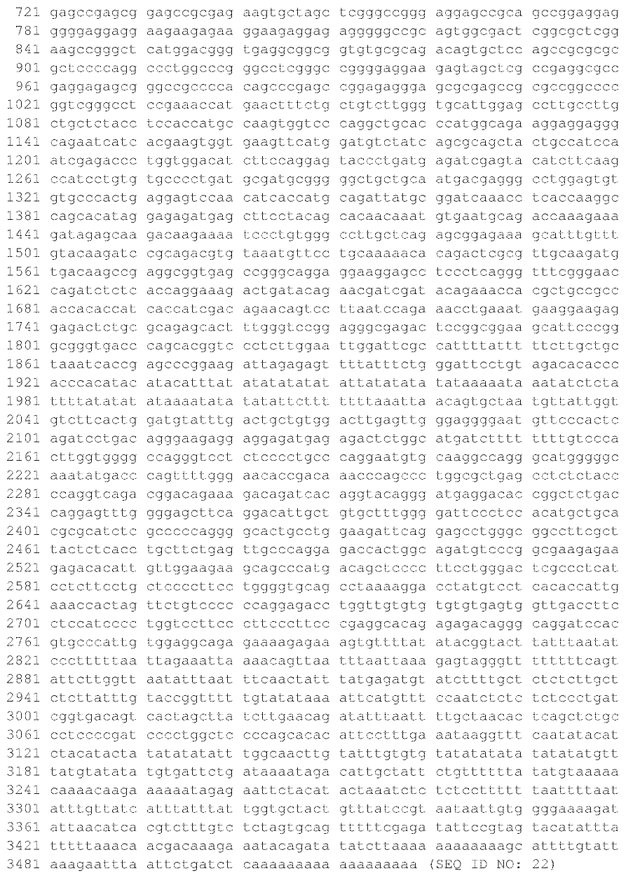

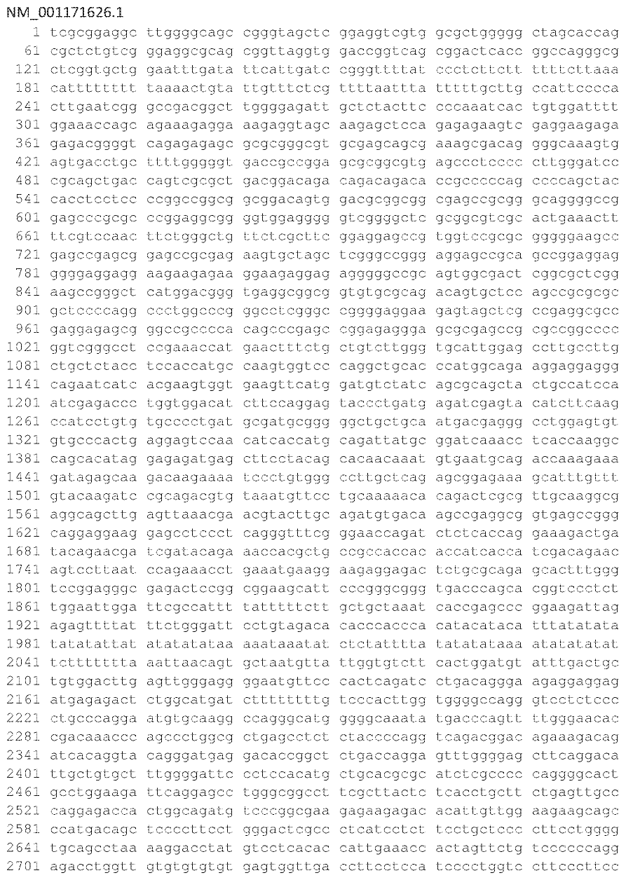

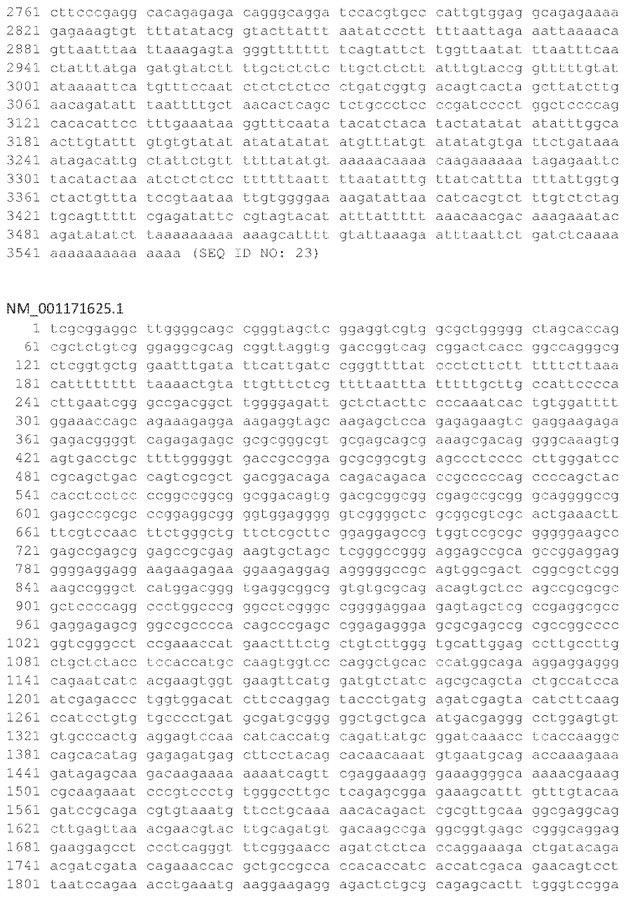

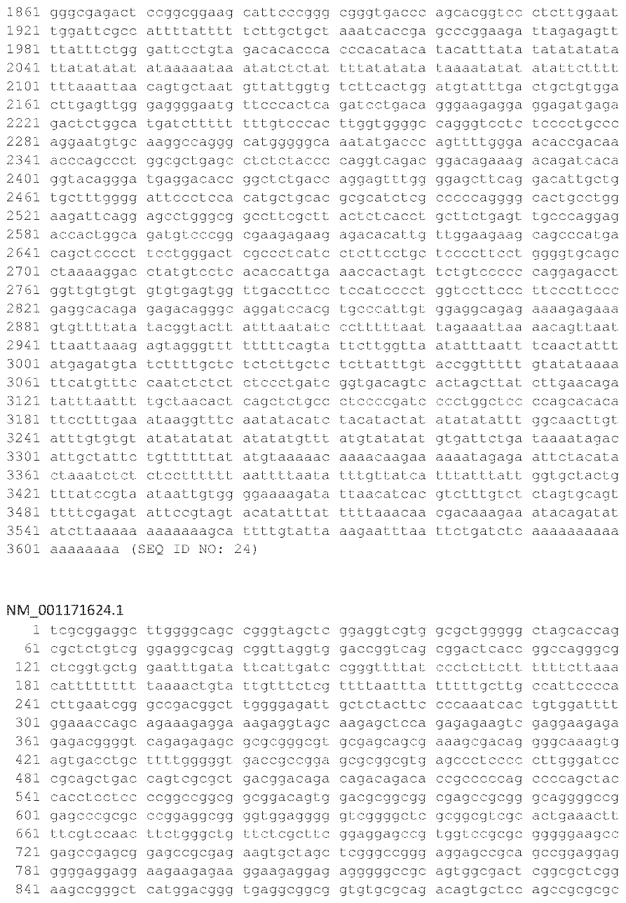

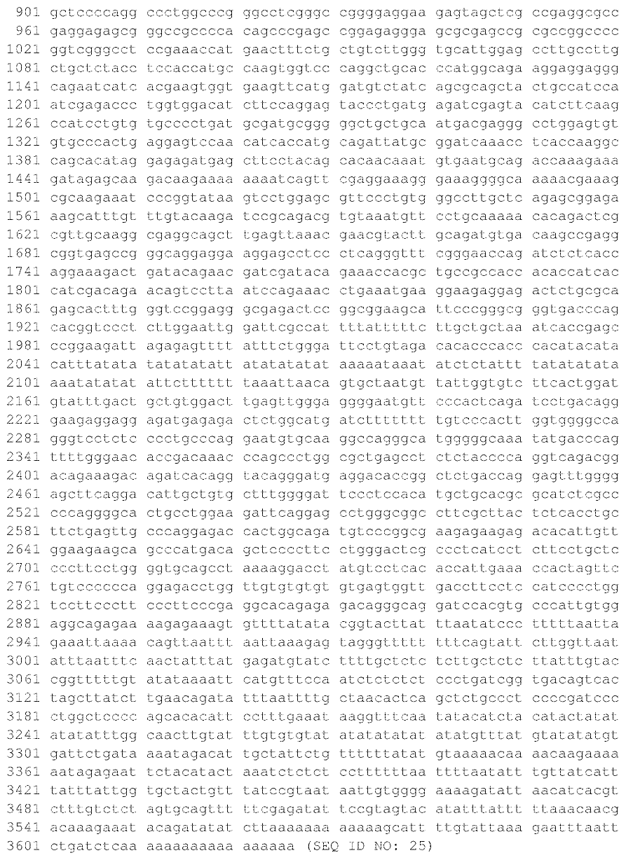

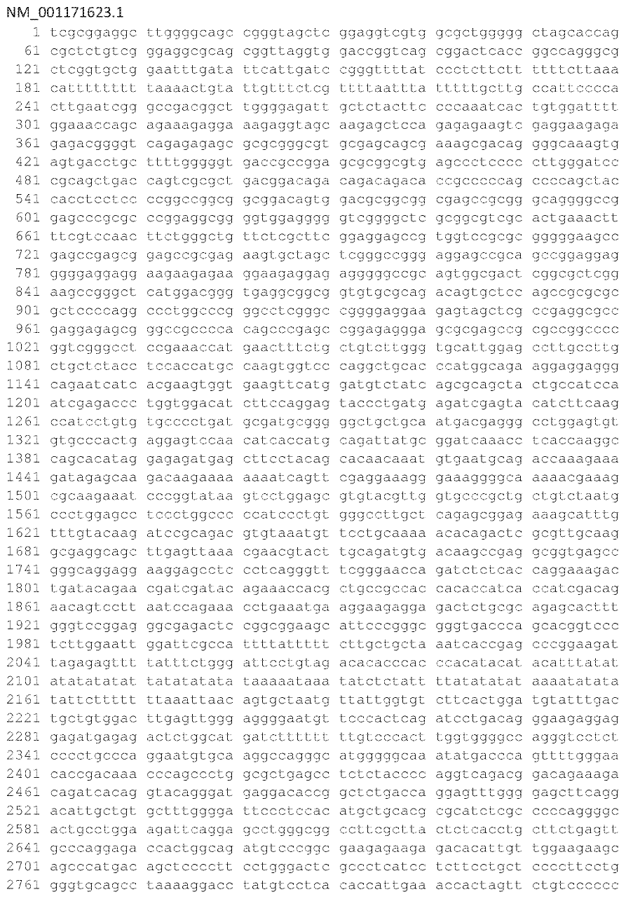

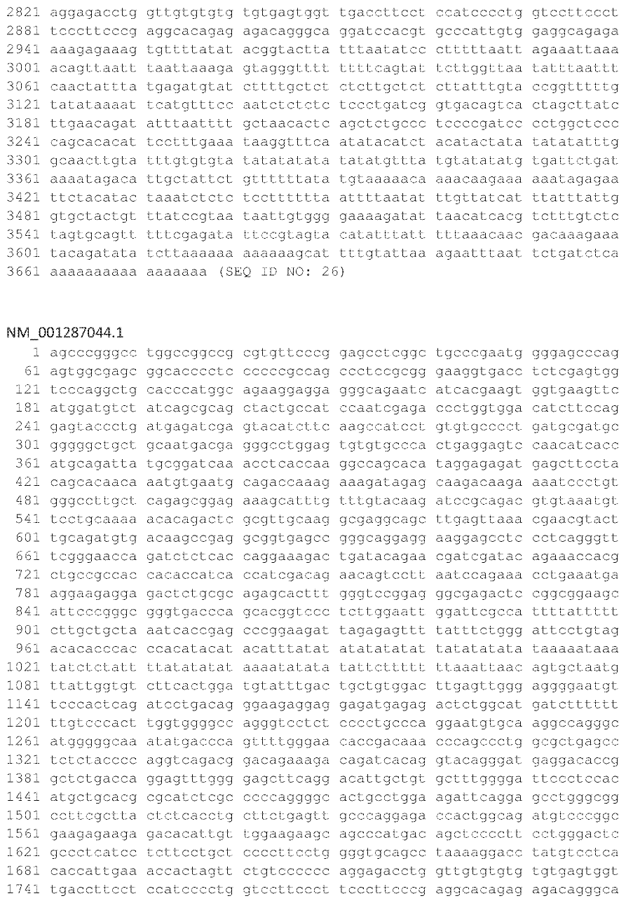

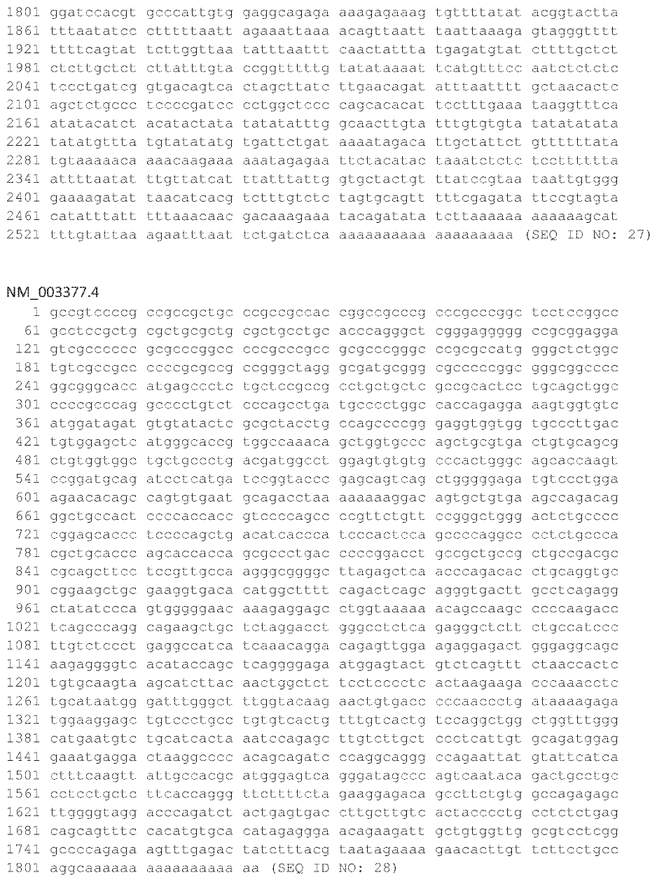

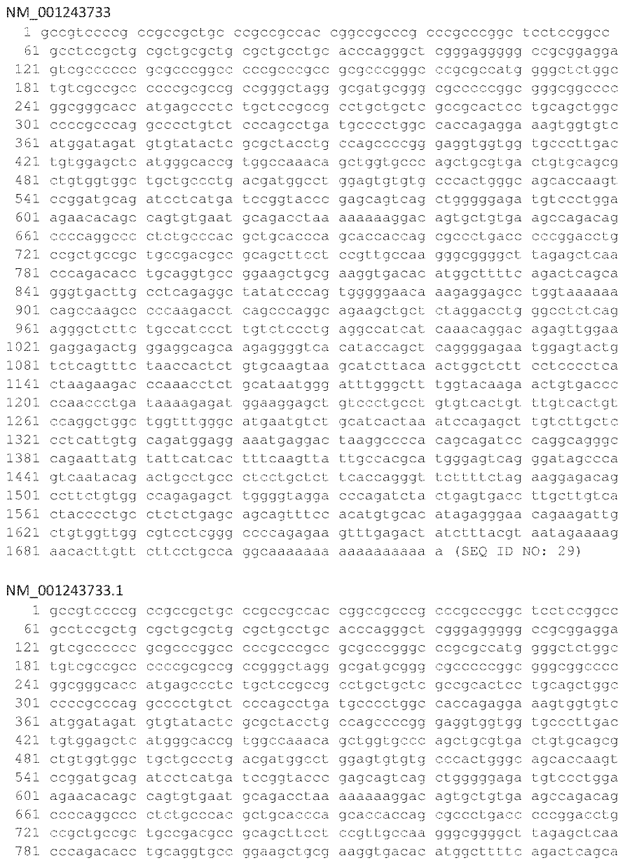

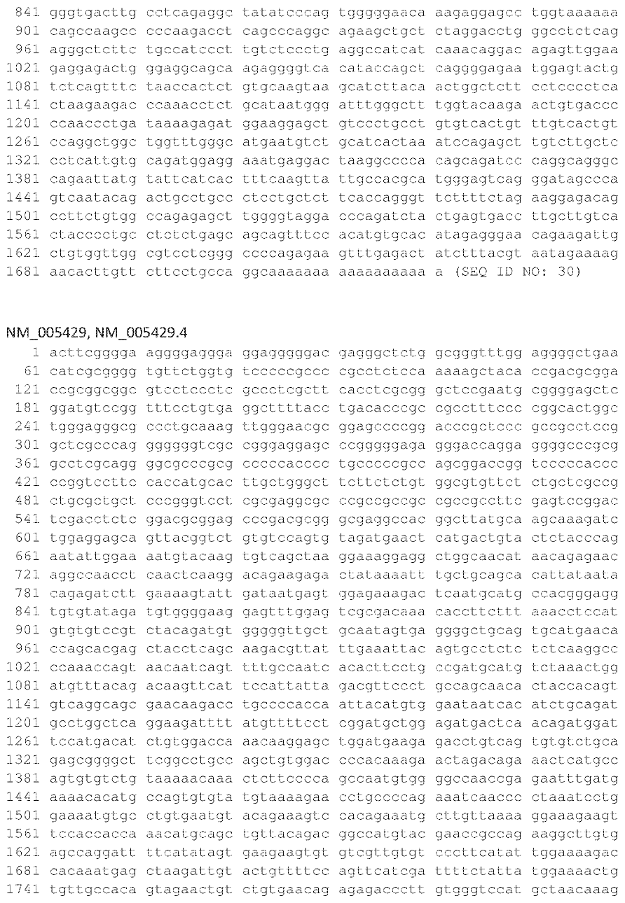

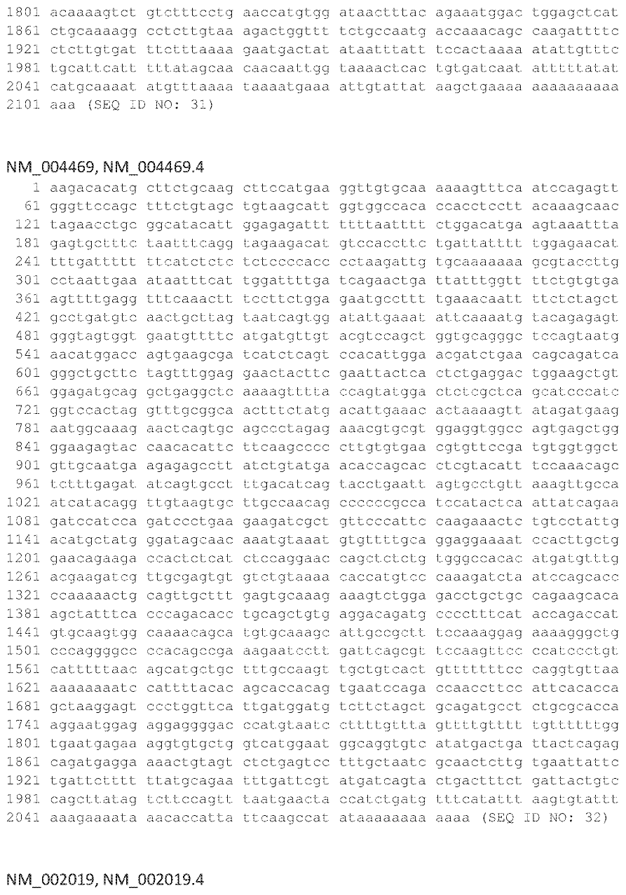

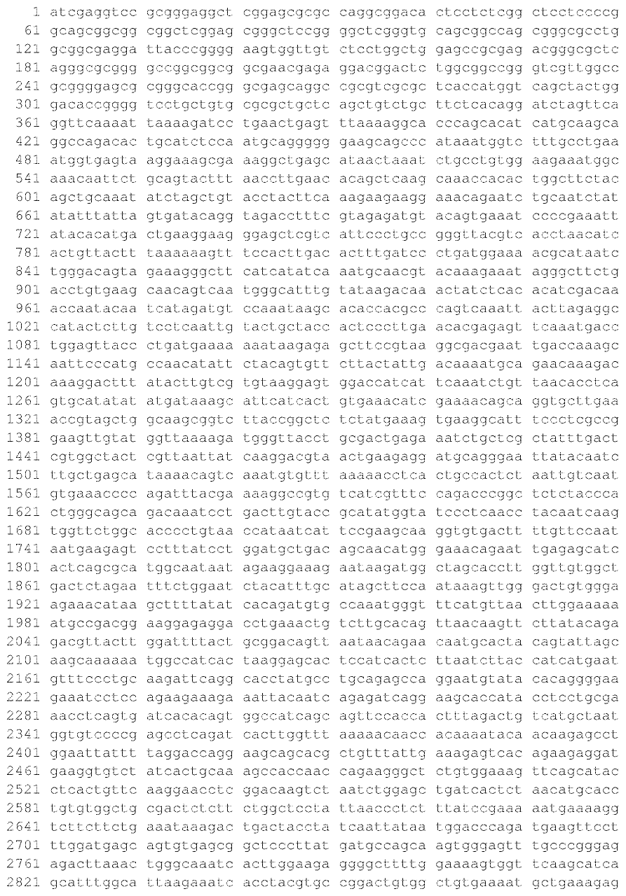

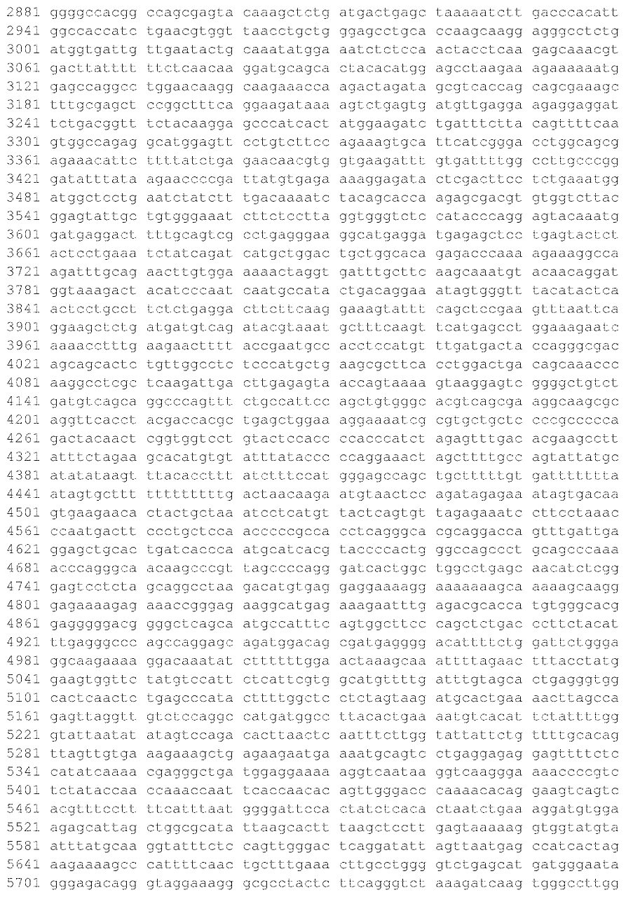

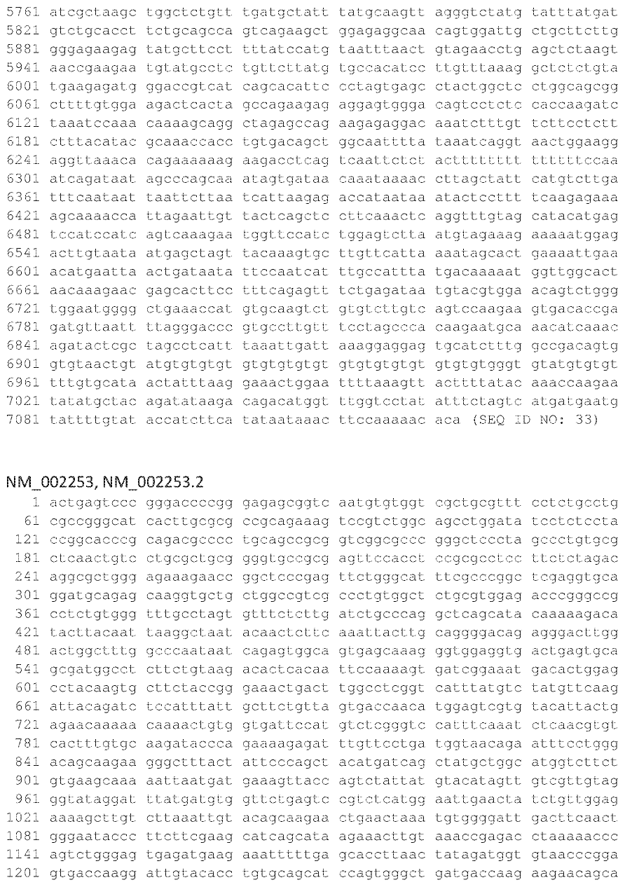

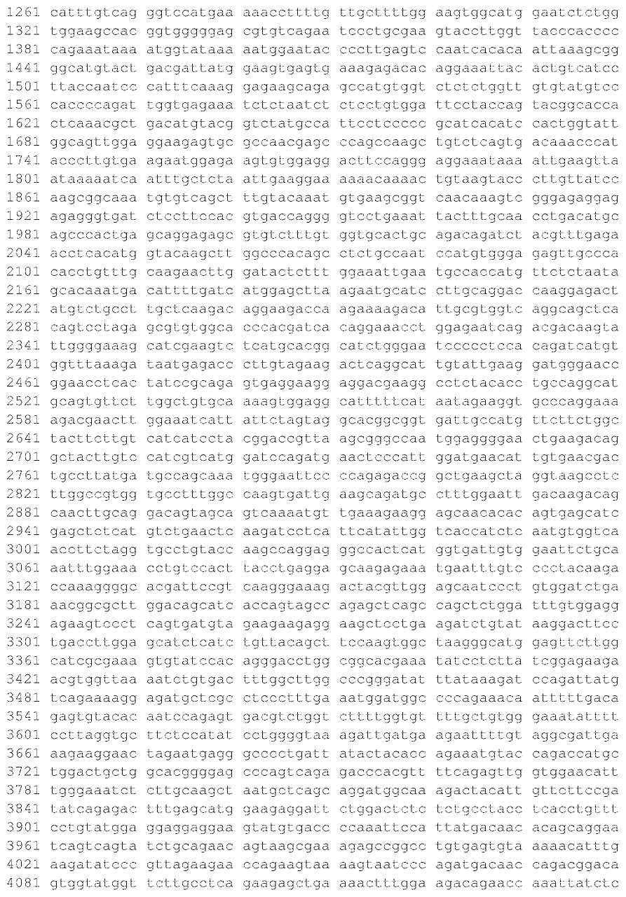

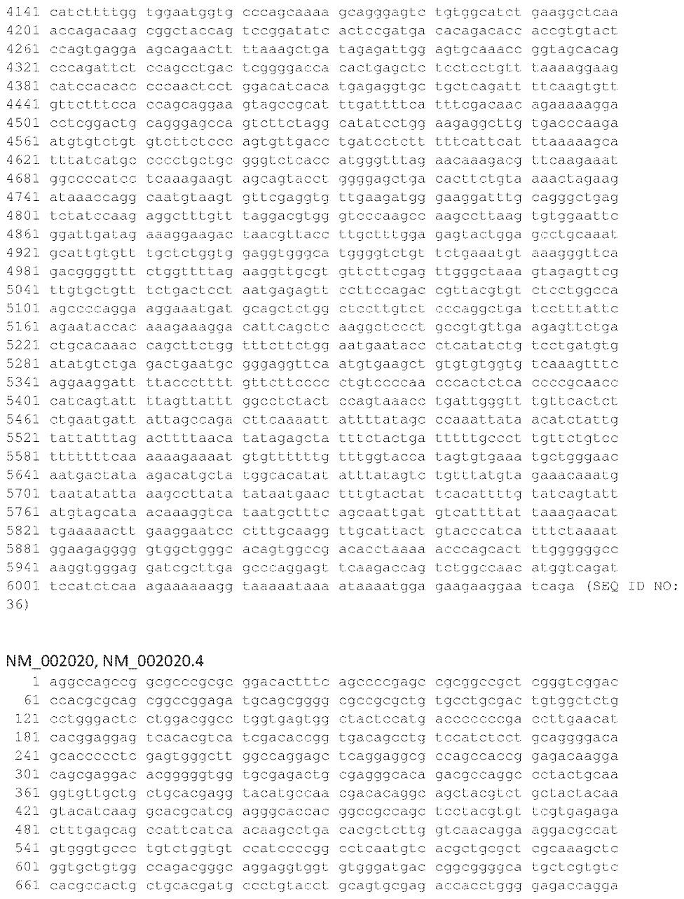

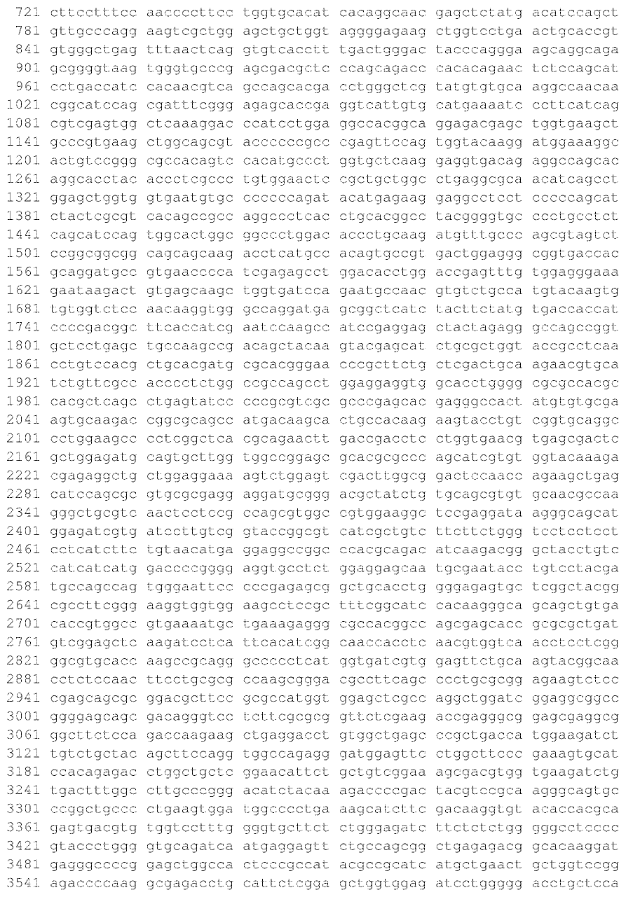

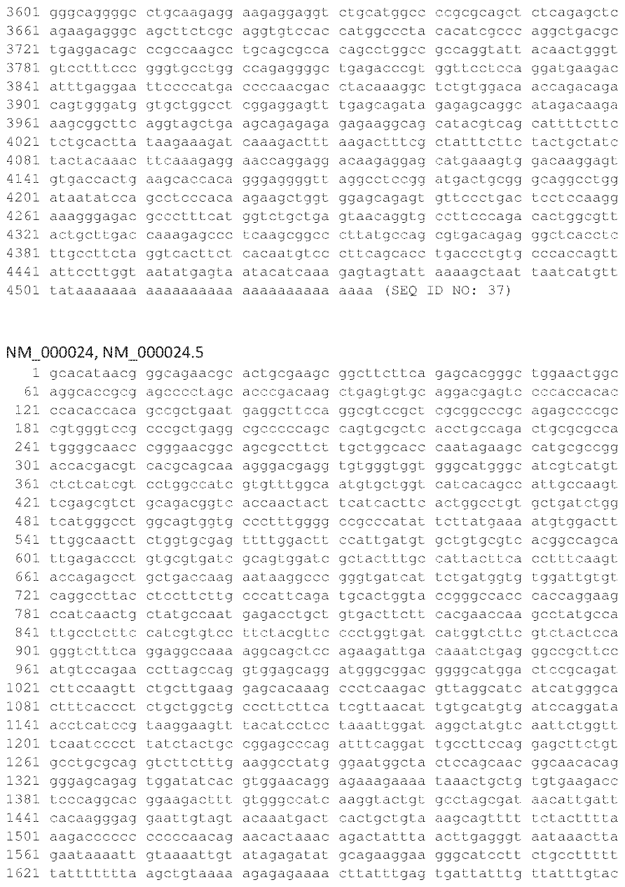

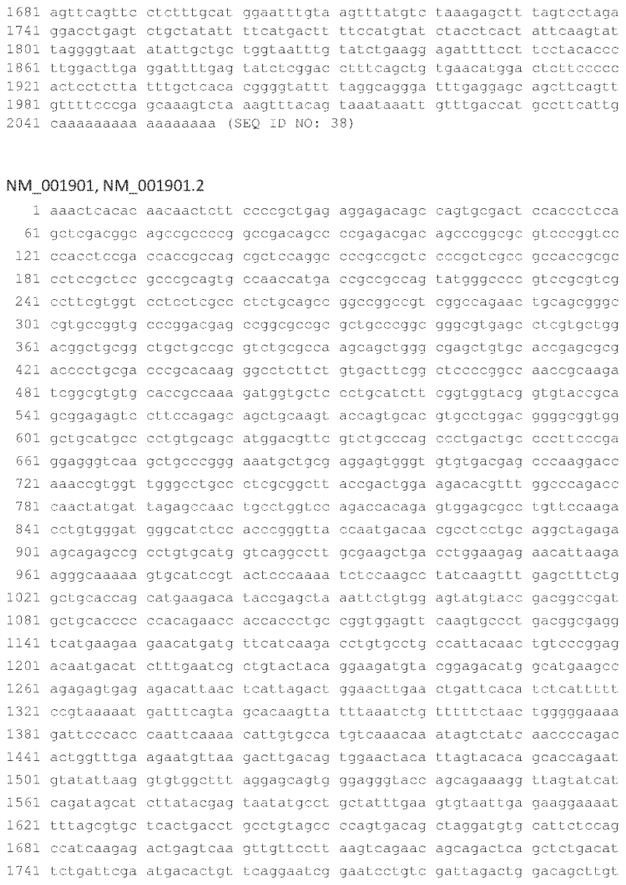

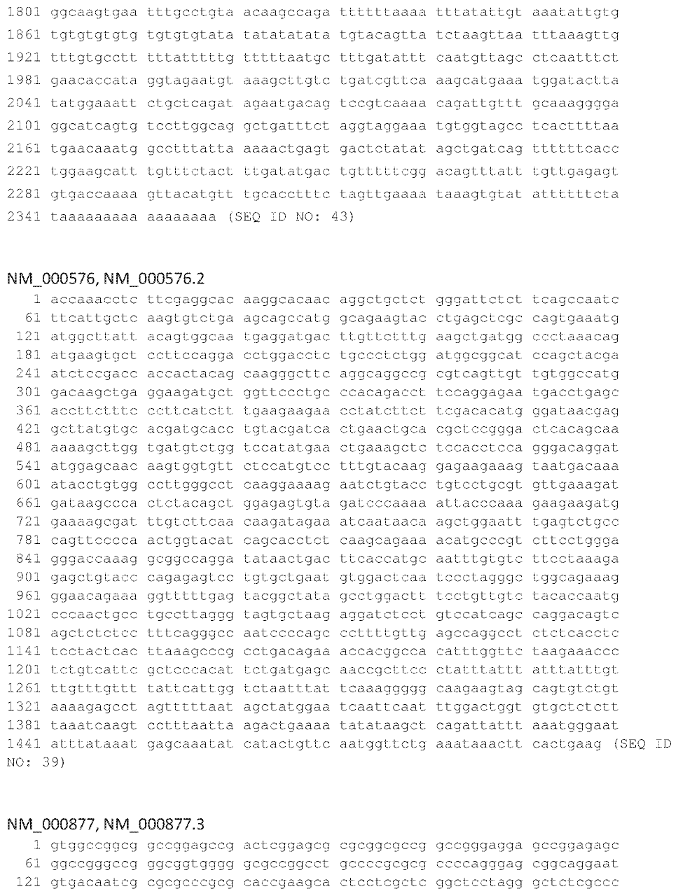

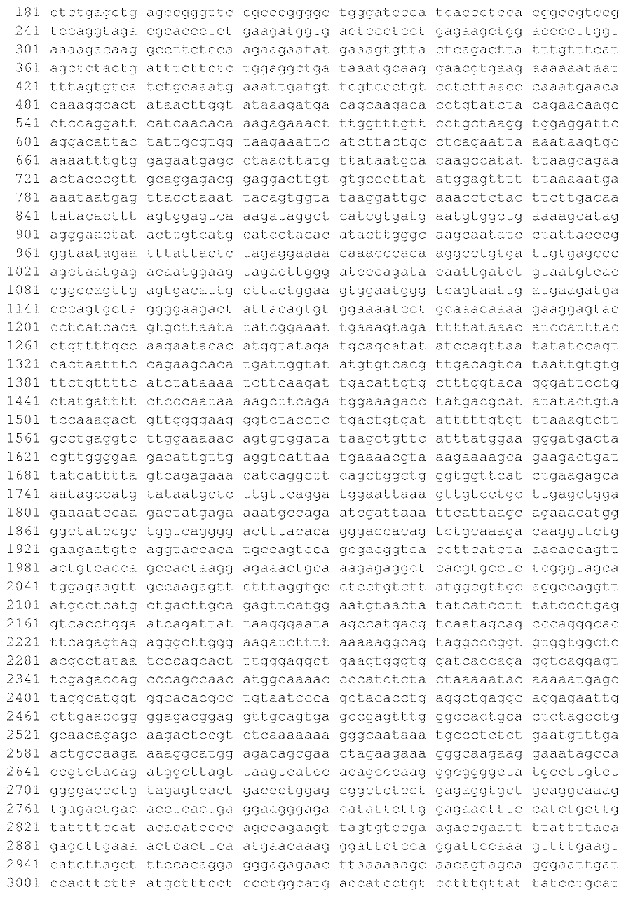

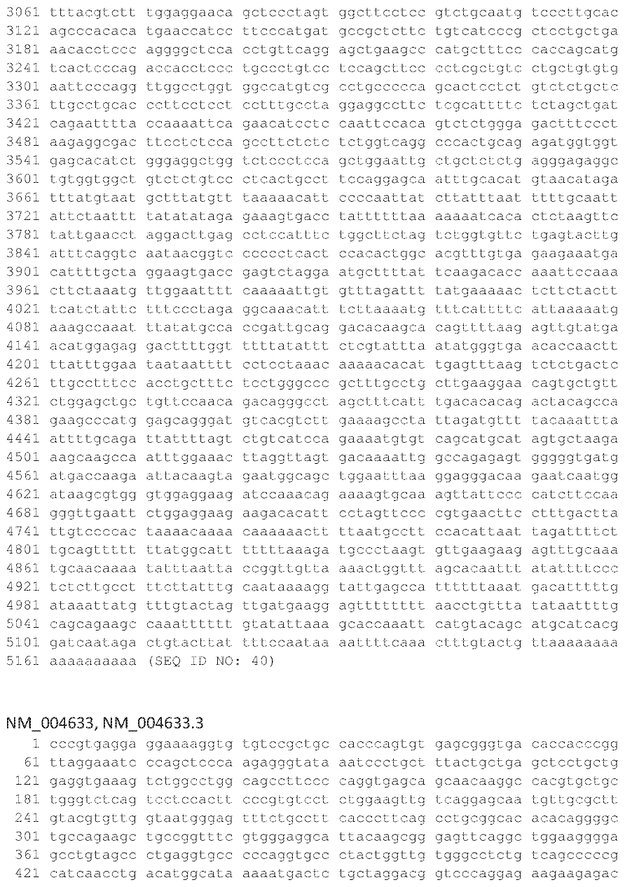

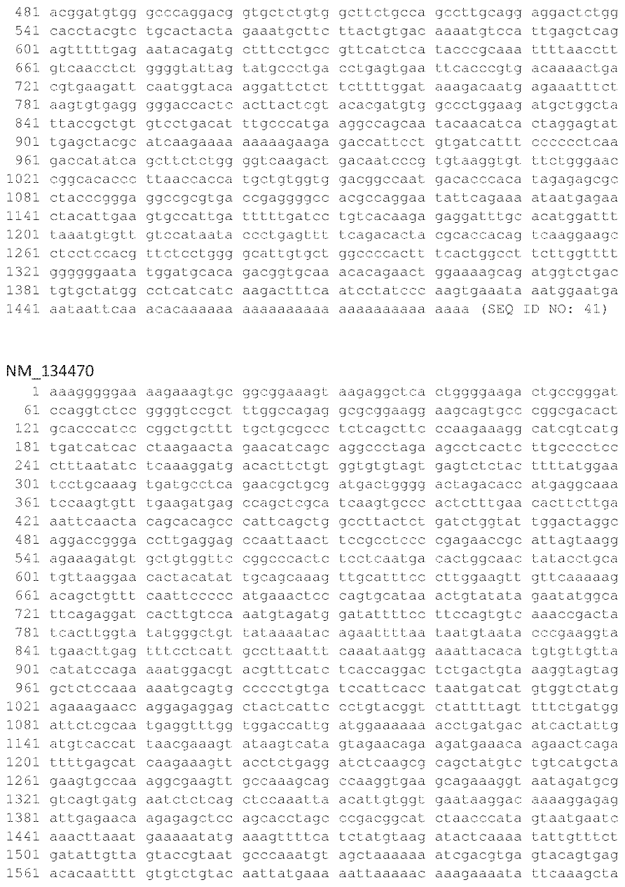

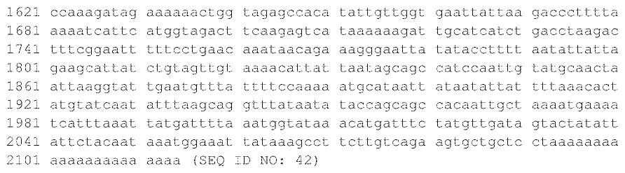

The nucleic acid sequences for mRNA for each of these is presented herein. For instance, the nucleotide sequence of SEQ ID NO: 34, set forth below is the human TNF-α cDNA sequence published by Nedwin, G. E. et al. (Nucleic Acids Res. 1985, 13, 6361-6373); and disclosed in Genbank accession number X02910. Other exemplary accession numbers and sequences are listed in Table 1 and provided in the accompanying sequence listing and in FIG. 7.

| Gene name for mRNA | Accession number | SEQ ID NO |
|---|---|---|
| Platelet-derived growth factor | NM_033023.4 | SEQ ID NO: 1 |
| subunit A (PDGFA) | NM_002607.5 | SEQ ID NO: 2 |
| Platelet-derived growth factor | NM_033016.3 | SEQ ID NO: 3 |
| subunit B (PDGFB) | NM_002608.3 | SEQ ID NO: 5 |
| Platelet-derived growth factor | NM_016205.2 | SEQ ID NO: 6 |
| subunit C (PDGFC) | | |
| Platelet-derived growth factor | NM_025208.4 | SEQ ID NO: 7 |
| subunit D (PDGFD) | NM_033135.3 | SEQ ID NO: 8 |
| Platelet-derived growth factor | NM_006206.4 | SEQ ID NO: 11 |
| receptor alpha (PDGFRA) | | |
| Platelet-derived growth factor | NM_002609.3 | SEQ ID NO: 12 |
| receptor beta (PDGFRB) | | |
| Platelet-derived growth factor | NM_006207.2 | SEQ ID NO: 13 |
| receptor like (PDGFRL) | | |
| Vascular endothelial growth | NM_001025366 | SEQ ID NO: 14 |
| factor A (VEGFA) | NM_001317010.1 | SEQ ID NO: 15 |
| | NM_001204384.1 | SEQ ID NO: 17 |
| | NM_001171630.1 | SEQ ID NO: 19 |
| | NM_001171629.1 | SEQ ID NO: 20 |
| | NM_001171628.1 | SEQ ID NO: 21 |
| | NM_001171627.1 | SEQ ID NO: 22 |
| | NM_001171626.1 | SEQ ID NO: 23 |
| | NM_001171625.1 | SEQ ID NO: 24 |
| | NM_001171624.1 | SEQ ID NO: 25 |
| | NM_001171623.1 | SEQ ID NO: 26 |
| | NM_001287044.1 | SEQ ID NO: 27 |
| Vascular endothelial growth | NM_003377.4 | SEQ ID NO: 28 |
| factor B (VEGFB) | NM_001243733 | SEQ ID NO: 29 |
| | NM_001243733.1 | SEQ ID NO: 30 |
| Vascular endothelial growth | NM_005429 & | SEQ ID NO: 31 |
| factor C (VEGFC) | NM_005429.4 | |
| Vascular endothelial growth | NM_004469 & | SEQ ID NO: 32 |
| factor D (VEGFD) | NM_004469.4 | |
| Vascular endothelial growth | NM_002019 & | SEQ ID NO: 33 |
| factor receptor-1 (VEGFR1) | NM_002019.4 | |
| Vascular endothelial growth | NM_002253 & | SEQ ID NO: 36 |
| factor receptor-2 (VEGFR2) | NM_002253.2 | |
| Vascular endothelial growth | NM_002020 & | SEQ ID NO: 37 |
| factor receptor-3 (VEGFR3) | NM_002020.4 | |
| beta-2 adrenergic receptor | NM_000024 & | SEQ ID NO: 38 |
| | NM_000024.5 | |
| Connective tissue growth | NM_001901 & | SEQ ID NO: 43 |
| factor (CTGF) | NM_001901.2 | |
| Interleukin 1, beta | NM_000576 & | SEQ ID NO: 39 |
| | NM_000576.2 | |
| Interleukin 1 receptor-1 | NM_000877 & | SEQ ID NO: 40 |
| (IL1R1) | NM_000877.3 | |
| Interleukin 1 receptor-2 | NM_004633 & | SEQ ID NO: 41 |
| (IL1R2) | NM_004633.3 | |
| Interleukin 1 receptor-3 | NM_134470 | SEQ ID NO: 42 |
| (IL1R3) | | |

The spherical nucleic acids (SNAs) described herein may be stable self-assembling nanostructures. For instance the nanostructure may be an antisense oligonucleotide of 18-19 nucleotides in length comprising TGGGAGTAGATGAGGTAC (SEQ ID NO: 4), wherein a hydrophobic group at the 3′ or 5′ terminus self-associates to form the core of the nanostructure in water or other suitable solvents. A hydrophobic group as used herein may include cholesterol, a cholesteryl or modified cholesteryl residue, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

The antisense oligonucleotides typically have a length of 15-20 bases, which is generally long enough to have one complementary sequence in the mammalian genome. Additionally, antisense compounds having a length of at least 12, typically at least 15 nucleotides in length hybridize well with their target mRNA. Thus, the antisense oligonucleotides of the invention are typically in a size range of 8-100 nucleotides, more preferably 12-50 nucleotides in length. In some embodiments of the invention the antisense oligonucleotides are of 18-19 nucleotides in length and comprise TGGGAGTAGATGAGGTAC (SEQ ID NO: 4). Antisense oligonucleotides that comprise SEQ ID NO: 4 may include further nucleotides on the 5′ and/or 3′ end of the oligonucleotide. However an antisense oligonucleotide that comprises SEQ ID NO: 4 and is limited to 18 nucleotides in length does not have any additional nucleotides on the 5′ or 3′ end of the molecule. Other non-nucleotide molecules may be linked covalently or non-covalently to the 5′ and/or 3′ end of those oligonucleotides.

In some instances, the antisense oligonucleotide is one of the following oligonucleotides: T-G-G-G-A-G-T-A-G-A-T-G-A-G-G-T-A-C (SEQ ID NO:4), mUmGmGmGmAmGmUmAmGmAmUmGmAmGmGmUmAmC (SEQ ID NO: 10, Oligo 3742), T*G*G*G*A*G*T*A*G*A*T*G*A*G*G*T*A*C (SEQ ID NO: 9, Oligo 3500), mUmGmGmGmAmGT*A*G*A*T*G*mAmGmGmUmAmC (SEQ ID NO: 16, Oligo 3534), and mU*mG*mG*mG*mA*mG*T*A*G*A*T*G*mA*mG*mG*mU*mA*mC (SEQ ID NO: 18, Oligo 3509) wherein—refers to a phosphodiester bond, * refers to a phosphorothioate bond, and m refers to an O-methyl.

In some embodiments, an oligonucleotide disclosed herein, such as an antisense oligonucleotide, is complementary to a target nucleic acid or target gene over the entire length of the oligonucleotide. In some embodiments, such antisense oligonucleotides are 99%, 95%, 90%, 85%, or 80% complementary to the target nucleic acid.

In some embodiments, an oligonucleotide disclosed herein is two to 100 nucleotides in length. In some embodiments, the oligonucleotide is three nucleotides in length, four nucleotides in length, five nucleotides in length, six nucleotides in length, seven nucleotides in length, eight nucleotides in length, nine nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, 41 nucleotides in length, 42 nucleotides in length, 43 nucleotides in length, 44 nucleotides in length, 45 nucleotides in length, 46 nucleotides in length, 47 nucleotides in length, 49 nucleotides in length, 50 nucleotides in length, 52 nucleotides in length, 54 nucleotides in length, 56 nucleotides in length, 58 nucleotides in length, 60 nucleotides in length, 62 nucleotides in length, 64 nucleotides in length, 66 nucleotides in length, 68 nucleotides in length, 70 nucleotides in length, 72 nucleotides in length, 74 nucleotides in length, 76 nucleotides in length, 78 nucleotides in length, 80 nucleotides in length, 82 nucleotides in length, 84 nucleotides in length, 86 nucleotides in length, 88 nucleotides in length, 90 nucleotides in length, 92 nucleotides in length, 94 nucleotides in length, 96 nucleotides in length, 100 nucleotides or more than 100 nucleotides in length, or any range or combination thereof. In some embodiments, the oligonucleotide is 18 nucleotides in length.

As used herein, “inhibiting the expression of a gene or activity” refers to a reduction or blockade of the expression of a gene or activity of a protein encoded by the gene due to administration of a SNA comprising an antisense disclosed herein, relative to the expression of a gene or of the activity of a protein in an untreated sample, control sample or baseline level and does not necessarily indicate a total elimination of expression or activity. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, ELISA, Western blotting, RIA, other immunoassays, and FACS or flow cytometric analysis.

As used herein, “lower”, “reduced”, “reduction” or “decrease” or “inhibit” means a decrease by at least 10% as compared to a baseline level of expression of the gene. For instance, a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a baseline level or reference level), or any decrease between 10-100% as compared to a baseline level or control level. When “decrease” or “inhibition” is used in the context of the level of expression or activity of a gene or a protein, it refers to a reduction in protein or nucleic acid level or activity in a cell, a cell extract, or a cell supernatant. For example, such a decrease may be due to reduced RNA stability, transcription, or translation, increased protein degradation, or RNA interference.

As used herein, “inhibiting the expression of a gene or activity” also refers to a reduction or blockade of the expression of a gene or activity of a protein encoded by the gene due to administration of a SNA comprising an antisense oligonucleotide disclosed herein relative to a reduction or blockade of the expression of a gene or activity of a protein encoded by the gene due to administration of a linear antisense oligonucleotide that is not in a SNA and does not necessarily indicate a total elimination of expression or activity. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, ELISA, Western blotting, RIA, other immunoassays, and FACS.

As used herein, “lower”, “reduced”, “reduction” or “decrease” or “inhibit” means a decrease by at least 10% in the expression of a gene after administration of an antisense in a SNA disclosed herein relative to the expression of a gene after administration of the linear antisense oligonucleotide that is not in a SNA. For instance, a decrease by at least about 20%, or at least about 30%, or at least about 40%, or at least about 50%, or at least about 60%, or at least about 70%, or at least about 80%, or at least about 90% or up to and including a 100% decrease, or any decrease between 10-100%. When “decrease” or “inhibition” is used in the context of the level of expression or activity of a gene or a protein, it refers to a reduction in protein or nucleic acid level or activity in a cell, a cell extract, or a cell supernatant. For example, such a decrease may be due to reduced RNA stability, transcription, or translation, increased protein degradation, or RNA interference.

As used herein, “phosphodiester internucleoside linkage” means a phosphate group that is covalently bonded to two adjacent nucleosides of a modified oligonucleotide. In some embodiments, an oligonucleotide is a strand of linked nucleosides connected via internucleoside linkages, wherein each nucleoside and internucleoside linkage may be modified or unmodified.

As used herein, the terms “internucleoside linkage” refers to a group or bond that forms a covalent linkage between adjacent nucleosides in an oligonucleotide. As used herein, a “modified internucleoside linkage” refers to any internucleoside linkage other than a naturally occurring, phosphate internucleoside linkage or phosphodiester linkage. Non-phosphate linkages are referred to herein as modified internucleoside linkages.

In some embodiments, the internucleoside linkage is a phosphorothioate linkage. As used herein, “phosphorothioate linkage” refers to a modified phosphate linkage in which one of the non-bridging oxygen atoms is replaced with a sulfur atom. A phosphorothioate internucleoside linkage is a modified internucleoside linkage. In some embodiments, all or 100% of the internucleoside linkages of an oligonucleotide described herein are phosphorothioate linkages. In some embodiments, less than all or less than 100% of the internucleoside linkages of an oligonucleotide described herein are phosphodiester linkages. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 99%, or 100% of the internucleoside linkages of an oligonucleotide disclosed herein, or any range or combination thereof, are phosphodiester linkages. In some embodiments, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 99%, or 100% of the internucleoside linkages of an oligonucleotide disclosed herein, or any ranges or combinations thereof, are phosphorothioate linkages.

In some embodiments, the phosphorothioate modified linkages are in a central region of the oligonucleotide. In some embodiments, the phosphorothioate modified linkages are at or near the 3′-end of the oligonucleotide. In some embodiments, the phosphorothioate modified linkages are at or near the 5′-end of the oligonucleotide.

In some embodiments, one of the internucleoside linkages, two of the internucleoside linkages, three of the internucleoside linkages, four of the internucleoside linkages, five of the internucleoside linkages, six of the internucleoside linkages, seven of the internucleoside linkages, eight of the internucleoside linkages, nine of the internucleoside linkages, 10 of the internucleoside linkages, 11 of the internucleoside linkages, 12 of the internucleoside linkages, 13 of the internucleoside linkages, 14 of the internucleoside linkages, 15 of the internucleoside linkages, 16 of the internucleoside linkages, 17 of the internucleoside linkages, 18 of the internucleoside linkages, 19 of the internucleoside linkages, 20 of the internucleoside linkages, 21 of the internucleoside linkages, 22 of the internucleoside linkages, 23 of the internucleoside linkages, 24 of the internucleoside linkages, 25 of the internucleoside linkages, 26 of the internucleoside linkages, 27 of the internucleoside linkages, 28 of the internucleoside linkages, 29 of the internucleoside linkages, 30 of the internucleoside linkages, 31 of the internucleoside linkages, 32 of the internucleoside linkages, 33 of the internucleoside linkages, 34 of the internucleoside linkages, 35 of the internucleoside linkages, 36 of the internucleoside linkages, 37 of the internucleoside linkages, 38 of the internucleoside linkages, 39 of the internucleoside linkages, 40 of the internucleoside linkages, 41 of the internucleoside linkages, 42 of the internucleoside linkages, 43 of the internucleoside linkages, 44 of the internucleoside linkages, 45 of the internucleoside linkages, 46 of the internucleoside linkages, 47 of the internucleoside linkages, 49 of the internucleoside linkages, 50 of the internucleoside linkages, 52 of the internucleoside linkages, 54 of the internucleoside linkages, 56 of the internucleoside linkages, 58 of the internucleoside linkages, 60 of the internucleoside linkages, 62 of the internucleoside linkages, 64 of the internucleoside linkages, 66 of the internucleoside linkages, 68 of the internucleoside linkages, 70 of the internucleoside linkages, 72 of the internucleoside linkages, 74 of the internucleoside linkages, 76 of the internucleoside linkages, 78 of the internucleoside linkages, 80 of the internucleoside linkages, 82 of the internucleoside linkages, 84 of the internucleoside linkages, 86 of the internucleoside linkages, 88 of the internucleoside linkages, 90 of the internucleoside linkages, 92 of the internucleoside linkages, 94 of the internucleoside linkages, 96 of the internucleoside linkages, 100 nucleotides or more than 100 of the internucleoside linkages, or any range or combination thereof of an oligonucleotide described herein, are phosphodiester linkages. In some embodiments, none of the internucleoside linkages of an oligonucleotide disclosed herein are phosphodiester linkages. In some embodiments, all of the internucleoside linkages of an oligonucleotide disclosed herein are phosphodiester linkages. In some embodiments, 11 of the internucleoside linkages of an oligonucleotide disclosed herein are phosphodiester linkages. In some embodiments, 17 of the internucleoside linkages of an oligonucleotide disclosed herein are phosphodiester linkages.

In some embodiments, the oligonucleotides disclosed herein comprise a phosphorothioate modification. In some embodiments, the oliggoncleotides comprise a backbone that comprises a phosphorothioate internucleoside linkage. In some embodiments, the phosphorothioate internucleoside linkage has a (Sp) stereochemical configuration. In some embodiments, the phosphorothioate internucleoside linkage has a (Rp) stereochemical configuration. In some embodiments, all the phosphorothioate internucleoside linkages have a (Sp) stereochemical configuration. In some embodiments, all the phosphorothioate internucleoside linkages have a (Rp) stereochemical configuration.

In some embodiments, the phosphorothioate internucleoside linkages have the same stereochemical configuration. In some embodiments, two to 30 of the phosphorothioate internucleoside linkages have the same stereochemical configuration. In some embodiments, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 of the phosphorothioate internucleoside linkages have the same stereochemical configuration. In some embodiments, the phosphorothioate internucleoside linkages have different stereochemical configurations.

In some embodiments, one of the internucleoside linkages, two of the internucleoside linkages, three of the internucleoside linkages, four of the internucleoside linkages, five of the internucleoside linkages, six of the internucleoside linkages, seven of the internucleoside linkages, eight of the internucleoside linkages, nine of the internucleoside linkages, 10 of the internucleoside linkages, 11 of the internucleoside linkages, 12 of the internucleoside linkages, 13 of the internucleoside linkages, 14 of the internucleoside linkages, 15 of the internucleoside linkages, 16 of the internucleoside linkages, 17 of the internucleoside linkages, 18 of the internucleoside linkages, 19 of the internucleoside linkages, 20 of the internucleoside linkages, 21 of the internucleoside linkages, 22 of the internucleoside linkages, 23 of the internucleoside linkages, 24 of the internucleoside linkages, 25 of the internucleoside linkages, 26 of the internucleoside linkages, 27 of the internucleoside linkages, 28 of the internucleoside linkages, 29 of the internucleoside linkages, 30 of the internucleoside linkages, 31 of the internucleoside linkages, 32 of the internucleoside linkages, 33 of the internucleoside linkages, 34 of the internucleoside linkages, 35 of the internucleoside linkages, 36 of the internucleoside linkages, 37 of the internucleoside linkages, 38 of the internucleoside linkages, 39 of the internucleoside linkages, 40 of the internucleoside linkages, 41 of the internucleoside linkages, 42 of the internucleoside linkages, 43 of the internucleoside linkages, 44 of the internucleoside linkages, 45 of the internucleoside linkages, 46 of the internucleoside linkages, 47 of the internucleoside linkages, 49 of the internucleoside linkages, 50 of the internucleoside linkages, 52 of the internucleoside linkages, 54 of the internucleoside linkages, 56 of the internucleoside linkages, 58 of the internucleoside linkages, 60 of the internucleoside linkages, 62 of the internucleoside linkages, 64 of the internucleoside linkages, 66 of the internucleoside linkages, 68 of the internucleoside linkages, 70 of the internucleoside linkages, 72 of the internucleoside linkages, 74 of the internucleoside linkages, 76 of the internucleoside linkages, 78 of the internucleoside linkages, 80 of the internucleoside linkages, 82 of the internucleoside linkages, 84 of the internucleoside linkages, 86 of the internucleoside linkages, 88 of the internucleoside linkages, 90 of the internucleoside linkages, 92 of the internucleoside linkages, 94 of the internucleoside linkages, 96 of the internucleoside linkages, 100 nucleotides or more than 100 of the internucleoside linkages, or any range or combination thereof of an oligonucleotide described herein are phosphorothioate linkages. In some embodiments, none of the internucleoside linkages of an oligonucleotide disclosed herein are phosphorothioate linkages. In some embodiments, all of the internucleoside linkages of an oligonucleotide disclosed herein are phosphorothioate linkages. In some embodiments, six of the internucleoside linkages of an oligonucleotide disclosed herein are phosphorothioate linkages. In some embodiments, 17 of the internucleoside linkages of an oligonucleotide disclosed herein are phosphorothioate linkages.

In some embodiments, the oligonucleotides are indirectly linked to the core, to one another and/or to one or more active agents (e.g., drug, compound, etc.) through a linker or more than one linker. In some embodiments, the oligonucleotides are directly linked to the core, to one another and/or to one or more active agents (e.g., drug, compound, etc.). In some embodiments, the linker is at the 5′ end or the 3′ end of the oligonucleotide. In some embodiments, all of the oligonucleotides are on the surface of the core. In some embodiments, all of the oligonucleotides are on the surface of the core with the 5′-end of the oligonucleotides facing outwards. In some embodiments, all of the oligonucleotides are on the surface of the core with the 3′-end of the oligonucleotides facing outwards. In some embodiments, the core does not include oligonucleotides encapsulated inside the core. In some embodiments, the oligonucleotides are not crosslinked. In some embodiments, at least 99%, 98%, 97%, 96%, 95%, 94%, 93%, 92%, 91% or 90% of the oligonucleotides are on the surface of the core. In some embodiments, the core includes less than 5%, 4%, 3%, 2%, or 1% of the oligonucleotides encapsulated inside the core.

In some embodiments, the linker is a non-nucleotidic linker. In some embodiments, the linker is selected from the group consisting of abasic residues (dSpacer), oligoethyleneglycol, triethyleneglycol, hexaethylenegylcol, alkane-diol, or butanediol. In some embodiments, the linker is a tocopherol, sphingolipid such as sphingosine, sphingosine phosphate, methylated sphingosine and sphinganine, ceramide, ceramide phosphate, 1-0 acyl ceramide, dihydroceramide, 2-hydroxy ceramide, sphingomyelin, glycosylated sphingolipid, sulfatide, ganglioside, phosphosphingolipid, and phytosphingosine or phytosphingosines of various lengths and saturation states and derivatives, phospholipid such as phosphatidylcholine, lysophosphatidylcholine, phosphatidic acid, lysophosphatidic acid, cyclic LPA, phosphatidylethanolamine, lysophosphatidylethanolamine, phosphatidylglycerol, lysophosphatidylglycerol, phosphatidylserine, lysophosphatidylserine, phosphatidylinositol, inositol phosphate, LPI, cardiolipin, lysocardiolipin, bis(monoacylglycero) phosphate, (diacylglycero) phosphate, ether lipid, diphytanyl ether lipid, and plasmogen or plasmalogens of various lengths, saturation states, and their derivatives, sterol such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipid, ether cationic lipid, lanthanide chelating lipid, A-ring substituted oxysterol, B-ring substituted oxysterol, D-ring substituted oxysterol, side-chain substituted oxysterol, double substituted oxysterol, cholestanoic acid or its derivatives, fluorinated sterol, fluorescent sterol, sulfonated sterol, phosphorylated sterol, and polyunsaturated sterol or polyunsaturated sterols of different lengths, saturation states, and their derivatives. In some embodiments, the linker is an oligoethyleneglycol. In some embodiments, the linker is a hexaethyleneglycol.

In some embodiments, the linker comprises a molecular species at the 3′ or 5′ termini of an oligonucleotide disclosed herein. In some embodiments, the molecular species is positioned in a core and the oligonucleotide extends radially from the core. Oligonucleotides of the nanostructure may be linked to one another or to the core either directly or indirectly through a covalent or non-covalent linkage or through a covalent or non-covalent interaction. In some embodiments, an oligonucleotide disclosed herein is attached to the core through a covalent interaction (e.g., thiol-gold interaction, liposome-oligonucleotide amide linkage or interaction, etc.). In some embodiments, an oligonucleotide disclosed herein is attached to the core through a non-covalent interaction (e.g., van der Waals interaction, ionic interaction or electrostatic interaction). In some embodiments, the non-covalent interaction is reversible. The linkage of one oligonucleotide to another oligonucleotide may be in addition to or alternatively to the linkage of that oligonucleotide to the core or liposomal core.

In some embodiments, the linker is between the molecular species and the oligonucleotide. In some embodiments, the linker comprises or consists of an oligonucleotide, a peptide, a polymer or an oligoethylene glycol (e.g., hexaethylene glycol or iSp18). In some embodiments, the linker does not comprise or does not consist of an oligonucleotide (e.g., non-nucleotidic linker), a peptide, a polymer or an oligoethylene. In some embodiments, the linker forms a covalent bond with a core, such as a gold-thiol bond that forms with a gold core.

In some embodiments, the linker is a double linker or a triple linker. In some embodiments, the double linker is two oligoethyleneglycols. In some embodiments, the two oligoethyleneglycols are hexaethylenegylcols. In some embodiments, the double linker or the triple linker is linked in between each single linker by a phosphodiester, phosphorothioate, methylphosphonate, or amide linkage.

In some embodiments, the SNA is administered at a dose of between 0.01 μg and 5 g. In some embodiments, a SNA disclosed herein is administered at a dose of or about 0.01 μg, of or about 0.1 μg, 0.2 μg, 0.3 μg, 0.4 μg, 0.5 μg, 0.6 μg, 0.7 μg, 0.8 μg, 0.9 μg, 1 μg, 1.1 μg, 1.2 μg, 1.3 μg, 1.4 μg, 1.5 μg, 1.6 μg, 1.7 μg, 1.8 μg, 1.9 μg, 2 μg, 2.1 μg, 2.2 μg, 2.3 μg, 2.4 μg, 2.5 μg, 2.6 μg, 2.7 μg, 2.8 μg, 2.9 μg, 3 μg, 3.1 μg, 3.2 μg, 3.3 μg, 3.4 μg, 3.5 μg, 3.6 μg, 3.7 μg, 3.8 μg, 3.9 μg, 4 μg, 4.5 μg, 5 μg, 5.5 μg, 6 μg, 6.5 μg, 7 μg, 7.5 μg, 8 μg, 8.5 μg, 9 μg, 9.5 μg, 10 μg, 10.5 μg, 11 μg, 11.5 μg, 12 μg, 12.5 μg, 30 μg, 13.5 μg, 40 μg, 14.5 μg, 50 μg, 15.5 μg, 60 μg, 16.5 μg, 70 μg, 70.5 μg, 18 μg, 18.5 μg, 19 μg, 19.5 μg, 20 μg, 20.5 μg, 21 μg, 21.5 μg, 22 μg, 22.5 μg, 23 μg, 23.5 μg, 24 μg, 24.5 μg, 25 μg, 25.5 μg, 26 μg, 26.5 μg, 27 μg, 27.5 μg, 28 μg, 28.5 μg, 29 μg, 29.5 μg, 30 μg, 30.5 μg, 31 μg, 31.5 μg, 32 μg, 32.5 μg, 33 μg, 33.5 μg, 34 μg, 34.5 μg, 35 μg, 35.5 μg, 36 μg, 36.5 μg, 37 μg, 37.5 μg, 38 μg, 38.5 μg, 39 μg, 39.5 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 65 μg, 70 μg, 75 μg, 80 μg, 85 μg, 90 μg, 95 μg, 100 μg, 110 μg, 120 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1000 μg, or any ranges or combinations thereof.

In some embodiments, the SNA is administered at a dose of or no more than 100 μg. In some embodiments, the SNA is administered at a dose of or no more than 200 μg. In some embodiments, the SNA is administered at a dose of or no more than 300 μg. In some embodiments, the SNA is administered at a dose of or no more than 400 μg.

In some embodiments, a SNA disclosed herein is administered at a dose of or about 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 30 mg, 13.5 mg, 40 mg, 14.5 mg, 50 mg, 15.5 mg, 60 mg, 16.5 mg, 70 mg, 70.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 65 mg, 70 mg, 75 mg, 80 mg, 85 mg, 90 mg, 95 mg, 100 mg, 110 mg, 120 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 1.5 g, 2 g, 2.5 g, 3 g, 3.5 g, 4 g or 5 g or more than 5 g, or any ranges or combinations thereof.

In some embodiments, the SNA is administered at a dose of 0.1 mg/kg of body weight, 0.2 mg/kg of body weight, 0.3 mg/kg of body weight, 0.4 mg/kg of body weight, 0.5 mg/kg of body weight, 0.6 mg/kg of body weight, 0.7 mg/kg of body weight, 0.8 mg/kg of body weight, 0.9 mg/kg of body weight, 1 mg/kg of body weight, 1.5 mg/kg of body weight, 2 mg/kg of body weight, 2.5 mg/kg of body weight, 3 mg/kg of body weight, 3.5 mg/kg of body weight, 4 mg/kg of body weight, 4.5 mg/kg of body weight, 5 mg/kg of body weight, 5.5 mg/kg of body weight, 6 mg/kg of body weight, 6.5 mg/kg of body weight, 7 mg/kg of body weight, 7.5 mg/kg of body weight, 8 mg/kg of body weight, 8.5 mg/kg of body weight, 9 mg/kg of body weight, 9.5 mg/kg of body weight, 10 mg/kg of body weight, 10.5 mg/kg of body weight, 11 mg/kg of body weight, 11.5 mg/kg of body weight, 12 mg/kg of body weight, 12.5 mg/kg of body weight, 13 mg/kg of body weight, 13.5 mg/kg of body weight, 14 mg/kg of body weight, 14.5 mg/kg of body weight, 15 mg/kg of body weight, 15.5 mg/kg of body weight, 16 mg/kg of body weight, 16.5 mg/kg of body weight, 17 mg/kg of body weight, 17.5 mg/kg of body weight, 18 mg/kg of body weight, 18.5 mg/kg of body weight, 19 mg/kg of body weight, 19.5 mg/kg of body weight, 20 mg/kg of body weight, 20.5 mg/kg of body weight, 21 mg/kg of body weight, 21.5 mg/kg of body weight, 22 mg/kg of body weight, 23 mg/kg of body weight, 24 mg/kg of body weight, 25 mg/kg of body weight, 26 mg/kg of body weight, 27 mg/kg of body weight, 28 mg/kg of body weight, 29 mg/kg of body weight, 30 mg/kg of body weight, 31 mg/kg of body weight, 32 mg/kg of body weight, 33 mg/kg of body weight, 34 mg/kg of body weight, 35 mg/kg of body weight, 36 mg/kg of body weight, 37 mg/kg of body weight, 38 mg/kg of body weight, 39 mg/kg of body weight, 40 mg/kg of body weight, 45 mg/kg of body weight, 50 mg/kg of body weight, 55 mg/kg of body weight, 60 mg/kg of body weight, 65 mg/kg of body weight, 70 mg/kg of body weight, 75 mg/kg of body weight, 80 mg/kg of body weight, 85 mg/kg of body weight, 90 mg/kg of body weight, 95 mg/kg of body weight, 100 mg/kg of body weight, 500 mg/kg of body weight, 1000 mg/kg of body weight or any range there of or combination thereof.

In some embodiments, the SNA is administered at a dose of 0.1 μg/kg of body weight, 0.2 μg/kg of body weight, 0.3 μg/kg of body weight, 0.4 μg/kg of body weight, 0.5 μg/kg of body weight, 0.6 μg/kg of body weight, 0.7 μg/kg of body weight, 0.8 μg/kg of body weight, 0.9 μg/kg of body weight, 1 μg/kg of body weight, 1.5 μg/kg of body weight, 2 μg/kg of body weight, 2.5 μg/kg of body weight, 3 μg/kg of body weight, 3.5 μg/kg of body weight, 4 μg/kg of body weight, 4.5 μg/kg of body weight, 5 μg/kg of body weight, 5.5 μg/kg of body weight, 6 μg/kg of body weight, 6.5 μg/kg of body weight, 7 μg/kg of body weight, 7.5 μg/kg of body weight, 8 μg/kg of body weight, 8.5 μg/kg of body weight, 9 μg/kg of body weight, 9.5 μg/kg of body weight, 10 μg/kg of body weight, 10.5 μg/kg of body weight, 11 μg/kg of body weight, 11.5 μg/kg of body weight, 12 μg/kg of body weight, 12.5 μg/kg of body weight, 13 μg/kg of body weight, 13.5 μg/kg of body weight, 14 μg/kg of body weight, 14.5 μg/kg of body weight, 15 μg/kg of body weight, 15.5 μg/kg of body weight, 16 μg/kg of body weight, 16.5 μg/kg of body weight, 17 μg/kg of body weight, 17.5 μg/kg of body weight, 18 μg/kg of body weight, 18.5 μg/kg of body weight, 19 μg/kg of body weight, 19.5 μg/kg of body weight, 20 μg/kg of body weight, 20.5 μg/kg of body weight, 21 μg/kg of body weight, 21.5 μg/kg of body weight, 22 μg/kg of body weight, 23 μg/kg of body weight, 24 μg/kg of body weight, 25 μg/kg of body weight, 26 μg/kg of body weight, 27 μg/kg of body weight, 28 μg/kg of body weight, 29 μg/kg of body weight, 30 μg/kg of body weight, 31 μg/kg of body weight, 32 μg/kg of body weight, 33 μg/kg of body weight, 34 μg/kg of body weight, 35 μg/kg of body weight, 36 μg/kg of body weight, 37 μg/kg of body weight, 38 μg/kg of body weight, 39 μg/kg of body weight, 40 μg/kg of body weight, 45 μg/kg of body weight, 50 μg/kg of body weight, 55 μg/kg of body weight, 60 μg/kg of body weight, 65 μg/kg of body weight, 70 μg/kg of body weight, 75 μg/kg of body weight, 80 μg/kg of body weight, 85 μg/kg of body weight, 90 μg/kg of body weight, 95 μg/kg of body weight, 100 μg/kg of body weight, 500 μg/kg of body weight, 1000 μg/kg of body weight or any ranges or combinations thereof.

In some embodiments, a dose is a therapeutic dose. As disclosed herein, the dose (e.g., a therapeutic dose) as it relates to administration of a SNA disclosed herein (e.g., without limitation the phrases “SNA is administered at a dose of,” “SNA is administered to the subject at a dose of,” “SNA is administered to the subject at a fixed dose,” “SNA is administered to the eye,” “the dose is administered,” etc.) refers to the total weight or total mass of active agent (i.e., total weight or total mass of oligonucleotides) that are part of the SNA and is being administered to the subject (e.g., subject with an eye disease or eye disorder).

In some embodiments, the dose is administered in a single dose. In some embodiments, the dose is divided equally among a specified total number of doses administered to the subject for the course of treatment for an eye disease or eye disorder. In some embodiments, the dose is not divided equally among a specified total number of doses administered to the subject for the course of treatment for an eye disease or eye disorder.

In some embodiments, the total number of doses is one dose, two doses, three doses, four doses, five doses, six doses, seven doses, eight doses, nine doses, 10 doses, 11 doses, 12 doses, 13 doses, 14 doses, 15 doses, 16 doses, 17 doses, 18 doses, 19 doses, 20 doses, 25 doses, 30 doses 35 doses, 40 doses, 45 doses, 50 doses, 55 doses, 60 doses, 65 doses, 70 doses, 75 doses, 80 doses, 85 doses, 90 doses, 95 doses, 100 doses, or more than 100 doses.

In some embodiments, a SNA disclosed herein is administered once a day, once every three days, once a week, once every two weeks, once every three weeks, once every four weeks, once every five weeks, once every six weeks, once every seven weeks, once every eight weeks, once every nine weeks, once every 10 weeks, once every 12 weeks, once every 18 weeks, once every 24 weeks, once a month, once every two months, once every three months, once every four months, once every five months, once every six months, once every seven months, once every eight months, once every nine months, once every 10 months, once every 11 months, once a year, once every two years, once every three years, once every four years, once every five years, when every six years, once every seven years, once every eight years, once every nine years, is everything use, once every 15 years, once every 20 years, once every 30 years, or once every 40 years, or once every 50 years or any combinations or ranges thereof.

In some embodiments, the SNA is administered once every three months. In some embodiments, the SNA is administered once every four months. In some embodiments, the SNA is administered once every five months. In some embodiments, the SNA is administered once every six months. In some embodiments, the SNA is administered once every seven months. In some embodiments, the SNA is administered once every eight months. In some embodiments, the SNA is administered once every nine months. In some embodiments, the SNA is administered once every 10 months. In some embodiments, the SNA is administered once every 11 months. In some embodiments, the SNA is administered once every 12 months.

In some embodiments, the eye disorder or eye disease is associated with ocular angiogenesis, ocular neovascularization, retinal edema, ocular hypertension, elevated intraocular pressure, retinal ischemia, posterior segment neovascularization, age-related macular degeneration, inflammation, macular edema, uveitis, dry eye, neovascular glaucoma, glaucoma, scleritis, diabetic retinopathy, retinitis pigmentosa, optic nerve injury, retinopathy of prematurity, retinal ganglion degeneration, macular degeneration, hereditary optic neuropathy, metabolic optic neuropathy, acute ischemic optic neuropathy, commotio retinae, retinal detachment, retinal tears, retinal holes, iatrogen or is retinopathy, myopia, conjunctivitis or eye cancer.

In some embodiments, the subject is a mammal. In some embodiments, the mammal is a vertebrate animal including, but not limited to, a mouse, rat, dog, cat, horse, cow, pig, sheep, goat, turkey, chicken, or monkey. In some embodiments, the subject is not a mammal, including but not limited to a fish (e.g., aquaculture species, salmon, etc.). In some embodiments, the subject is human.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with an eye disease or eye disorder disclosed herein. Treatment is generally “effective” if one or more symptoms or clinical markers are reduced or otherwise improved as determined using standard techniques or procedures available to one of ordinary skill in the art. Alternatively, treatment is “effective” if the progression of an eye disease or eye disorder disclosed herein is reduced or halted, as determined by one of ordinary skill in the art. That is, “treatment” includes not just the improvement of symptoms or markers, but can also include a cessation or at least slowing of progress or worsening of symptoms that would be expected in absence of treatment. The term “treatment” of a disease also includes providing relief from the symptoms or side-effects of the disease (including palliative treatment).

The terms “significantly different than,” “statistically significant,” and similar phrases refer to comparisons between data or other measurements, wherein the differences between two compared individuals or groups are evidently or reasonably different to the trained observer, or the differences are statistically significant (if the phrase includes the term “statistically” or if there is some indication of statistical test, such as a p-value, or if the data, when analyzed, produce a statistical difference by standard statistical tests known in the art).

Effective amounts, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range or tissue concentration range that includes the IC₅₀ (i.e., the concentration of the active ingredient, which achieves a half-maximal inhibition of symptoms) as determined in cell culture, or in an appropriate animal model. Levels in plasma or in a tissue can be measured, for example, by high performance liquid chromatography. The effects of any particular dosage can be monitored by a suitable bioassay. The dosage can be determined by a physician and adjusted, as necessary, to suit observed effects of the treatment.

In some embodiments, the oligonucleotide is linked to a molecular species at the 3′ or 5′ terminus of the oligonucleotide through a linker. In some embodiments, the molecular species is linked to the oligonucleotide at the 5′ end of the oligonucleotide. In some embodiments, the molecular species is linked to the oligonucleotide at the 3′ end of the oligonucleotide. In some embodiments, a first molecular species is linked to the oligonucleotide at the 5′ end of the oligonucleotide and a second molecular species is linked to the oligonucleotide at the 3′ end of the oligonucleotide. In some embodiments, the first molecular species and the second molecular species are the same. In some embodiments, the first molecular species and the second molecular species are not the same. In some embodiments, the oligonucleotide is linked to a molecular species at the 3′ and/or 5′ terminus of the oligonucleotide without a linker.

In some embodiments, the molecular species is a hydrophobic group. In some embodiments, the hydrophobic group is selected from the group consisting of cholesterol, a cholesteryl, a modified cholesteryl residue, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid, taurocholic acid, deoxycholate, oleyl litocholic acid, oleoyl cholenic acid, glycolipids, phospholipids, sphingolipids, isoprenoids, vitamins, saturated fatty acid, unsaturated fatty acid, fatty acid ester, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dye, Hoechst 33258 dye, psoralen and ibuprofen. In some embodiments, the hydrophobic group is selected from the group consisting of a steroid, vitamin E, triglyceride, Cy3 and Cy5. In some embodiments, the hydrophobic group is cholesterol.

In some embodiments, a SNA disclosed herein is administered to the eye through intravitreal injection (IVI), scleral plug, subconjunctival implant, suprachoroidal implant, suprascleral injection, intravitreal implant, an oculex product or another intraocular implant, ocular product, or ocular procedure that delivers a SNA disclosed herein to the eye, a region of the eye, or a part of the eye, which is known to one of ordinary skill in the art. In some embodiments, a SNA disclosed herein is administered through intravitreal, subconjunctival, or periocular routes of administration. In some embodiments, a SNA disclosed herein is delivered in a sustained-release formulation, a biodegradable implant, a non-biodegradable implant, or through iontophoresis. In some embodiments, a SNA disclosed herein is administered through a parenteral route of administration. In some embodiments, a SNA disclosed herein is administered through a subcutaneous, intraperitoneal, intravenous, intradermal, or intramuscular route of administration. In some embodiments, a SNA disclosed herein is administered through subcutaneous injection, intraperitoneal injection, intravenous injection, or intramuscular injection. In some embodiments, a SNA disclosed herein is administered through a systemic route of administration.

In some embodiments, a SNA disclosed herein comprises a core. In some embodiments, the core is a liposomal core. A liposomal core or liposome core is used interchangeably herein and refers to a centrally located core compartment formed by a component of the lipids or phospholipids that form a lipid bilayer.

“Liposomes” are artificial, self closed vesicular structure of various sizes and structures, where one or several membranes encapsulate an aqueous core. Most typically liposome membranes are formed from lipid bilayers, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives. The lipid bilayer is composed of two layers of lipid molecules. Each lipid molecule in a layer is oriented substantially parallel to adjacent lipid molecules in the lipid layer, and two layers that form a bilayer have the polar ends of their molecules exposed to the aqueous phase and the non-polar ends adjacent to each other. The central aqueous region of the liposomal core may be empty or filled fully or partially with water, an aqueous solution or emulsion, oligonucleotides, or other therapeutic or diagnostic agents.

In some embodiments, the core comprises or consists of an inorganic core. In some embodiments, the core comprises or consists of a metal core. Non-limiting examples of an inorganic core or a metal core include a core comprising or consisting of gold, silver, platinum, aluminum, palladium, copper, cobalt, indium, nickel, paramagnetic iron oxide and mixtures thereof. In some embodiments, the core comprises or consists of gold. In some embodiments, a nanostructure disclosed herein is degradable. In some embodiments, the core is a solid core. In some embodiments, the core is a hollow core. In some embodiments, a nanostructure or core disclosed herein comprises a semiconductor or magnetic material. In some embodiments, the core is a liposomal core.

In some embodiments, the core is a solid or hollow core. In some embodiments, the core is about 15 nm to about 30 nm in diameter. In some embodiments, the core is 15 nm to 30 nm in diameter. In some embodiments, the core, such as a liposomal core or liposome core, as used interchangeably herein, of a SNA disclosed herein, has a diameter of or about 10 to of or about 150 nm. In some embodiments, the diameter of the core is from of or about 15 nm to of or about 100 nm, of or about 20 nm to of or about 100 nm, of or about 25 nm to of or about 100 nm, of or about 15 nm to of or about 50 nm, of or about 20 nm to of or about 50 nm, of or about 10 nm to of or about 70 nm, of or about 15 nm to of or about 70 nm, of or about 20 nm to of or about 70 nm, of or about 10 nm to of or about 30 nm, of or about 15 nm to of or about 30 nm, of or about 20 nm to of or about 30 nm, of or about 10 nm to of or about 40 nm, of or about 15 nm to of or about 40 nm, of or about 20 nm to of or about 40 nm, of or about 30 nm to of or about 40 nm, of or about 10 nm to of or about 80 nm, of or about 15 nm to of or about 80 nm, or of or about 20 nm to of or about 80 nm. In some embodiments, the core has a diameter of about 20 nm to about 40 nm. In some embodiments, the core has a diameter of 20 nm to 40 nm. In some embodiments, the core has a diameter of about 30 nm to about 40 nm. In some embodiments, the core has a diameter of 30 nm to 40 nm. In some embodiments, the core has a diameter of or about 30 nm. In some embodiments, the core has a diameter of 30 nm. In some embodiments, the core has a diameter of or about 20 nm. In some embodiments, the core has a diameter of 20 nm.

In some embodiments, the core, such as a liposomal core or liposome core, as used interchangeably herein, of a SNA disclosed herein has a diameter of or about 10 nm, 15 nm, 20 nm, 25 nm, 30 nm, 35 nm, or 40 nm. In some embodiments, the core has a diameter of less than about 10 nm, of less than about 15 nm, of less than about 20 nm, of less than about 25 nm, of less than about 30 nm, of less than about 35 nm, or of less than about 40 nm. In some embodiments, the core has a diameter of about 10 nm, about 15 nm, about 20 nm, about 25 nm, about 30 nm, about 35 nm, or about 40 nm. In some embodiments, the core has a diameter of about 30 nm.

In some embodiments, a plurality of SNAs or a population of SNAs is used in any of the methods disclosed herein. In some embodiments, the plurality of SNAs or the population of SNAs each comprise a core, such as a liposomal core or liposome core, as used herein, which when taken together, have a mean diameter of or about 15 nm to of or about 30 nm. In some embodiments, the mean diameter of the cores, such as the liposomal cores or liposomal cores, of the plurality of SNAs or population of SNAs disclosed herein is of or about 10 to of or about 150 nm. In some embodiments, the mean diameter of the cores is from of or about 15 nm to of or about 100 nm, of or about 20 nm to of or about 100 nm, of or about 25 nm to of or about 100 nm, of or about 15 nm to of or about 50 nm, of or about 20 nm to of or about 50 nm, of or about 10 nm to of or about 70 nm, of or about 15 nm to of or about 70 nm, of or about 20 nm to of or about 70 nm, of or about 10 nm to of or about 30 nm, of or about 15 nm to of or about 30 nm, of or about 20 nm to of or about 30 nm, of or about 10 nm to of or about 40 nm, of or about 15 nm to of or about 40 nm, of or about 20 nm to of or about 40 nm, of or about 10 nm to of or about 80 nm, of or about 15 nm to of or about 80 nm, or of or about 20 nm to of or about 80 nm.

In some embodiments, a SNA disclosed herein has a diameter of about 15 nm to 30 nm. In some embodiments, a SNA disclosed herein has a diameter of or about 10 to of or about 150 nm. In some embodiments, a SNA disclosed herein has a diameter of or about 15 nm to of or about 100 nm, of or about 20 nm to of or about 100 nm, of or about 25 nm to of or about 100 nm, of or about 15 nm to of or about 50 nm, of or about 20 nm to of or about 50 nm, of or about 10 nm to of or about 70 nm, of or about 15 nm to of or about 70 nm, of or about 20 nm to of or about 70 nm, of or about 10 nm to of or about 30 nm, of or about 15 nm to of or about 30 nm, of or about 20 nm to of or about 30 nm, of or about 10 nm to of or about 40 nm, of or about 15 nm to of or about 40 nm, of or about 20 nm to of or about 40 nm, of or about 10 nm to of or about 80 nm, of or about 15 nm to of or about 80 nm, or of or about 20 nm to of or about 80 nm.

In some embodiments, a SNA disclosed herein has a diameter of 10 nm, 15 nm, 20 nm, 25 nm, 30 nm, 35 nm, 40 nm, 50 nm, 60 nm, 70 nm, 80 nm, 90 nm, 100 nm, 150 nm, 200 nm, 250 nm, 300 nm, 350 nm, 400 nm, 450 nm, or 500 nm. In some embodiments, a SNA disclosed herein has a diameter of less than about 10 nm, of less than about 15 nm, of less than about 20 nm, of less than about 25 nm, of less than about 30 nm, of less than about 35 nm, of less than about 40 nm, of less than about 50 nm, of less than about 60 nm, of less than about 70 nm, of less than about 80 nm, of less than about 90 nm, of less than about 100 nm, of less than about 150 nm, of less than about 200 nm, of less than about 250 nm, of less than about 300 nm, of less than about 350 nm, of less than about 400 nm, of less than about 450 nm, or of less than about 500 nm. In some embodiments, a SNA disclosed herein has a diameter of about 10 nm, about 15 nm, about 20 nm, about 25 nm, about 30 nm, about 35 nm, about 40 nm, about 50 nm, about 60 nm, about 70 nm, about 80 nm, about 90 nm, about 100 nm, about 150 nm, about 200 nm, about 250 nm, about 300 nm, about 350 nm, about 400 nm, about 450 nm, or about 500 nm.

In some embodiments, a plurality of SNAs or a population of SNAs disclosed herein, when taken together, have a mean diameter of or about 15 nm to of or about 30 nm. In some embodiments, the mean diameter is of or about 10 to of or about 150 nm. In some embodiments, the mean diameter is from of or about 15 nm to of or about 100 nm, of or about 20 nm to of or about 100 nm, of or about 25 nm to of or about 100 nm, of or about 15 nm to of or about 50 nm, of or about 20 nm to of or about 50 nm, of or about 10 nm to of or about 70 nm, of or about 15 nm to of or about 70 nm, of or about 20 nm to of or about 70 nm, of or about 10 nm to of or about 30 nm, of or about 15 nm to of or about 30 nm, of or about 20 nm to of or about 30 nm, of or about 10 nm to of or about 40 nm, of or about 15 nm to of or about 40 nm, of or about 20 nm to of or about 40 nm, of or about 10 nm to of or about 80 nm, of or about 15 nm to of or about 80 nm, or of or about 20 nm to of or about 80 nm.

In some embodiments, the ratio of oligonucleotides to the diameter of the core of a SNA disclosed herein in nm (e.g., liposomal core of a SNA, gold core of a SNA, etc.) disclosed herein is 30:20 (i.e., 30 oligonucleotide molecules per 20 nm diameter of SNA core), 6:1, 30:5, 3:1, 30:10, 15:2, 30:15, 3:2, 30:20, 6:5, 30:25, 1:1, 6:7, 30:35, 3:4, 30:40, 2:3, 30:45, 3:5, 30:50, 6:11, 30:55, 1:2, 6:13, 30:65, 3:7, 30:70, 2:5, 30:75, 3:8, 30:80, 6:17, 30:85, 1:3, 6:19, 30:95, 3:10, 30:100, 1:5, 3:20, 30:200, 1:10, 30:300, 1:4, 1:2, 3:4, 5:4, 1:1, 7:4, 2:1, 9:4, 5:2, 11:4, 3:1, 13:4, 7:2, 15:4, 4:1, 17:4, 19:4, 5:1, 10:1, or 15:1 or any range or combination thereof.

In some embodiments, the oligonucleotides may be positioned on the exterior of the core, within the walls of the core and/or in the center of the core. An oligonucleotide that is positioned on the core is typically referred to as attached to the core. In some embodiments, the plurality of oligonucleotides in the SNA are linked to the exterior of the core. The oligonucleotides of the oligonucleotide shell may be oriented in a variety of directions. In some embodiments the oligonucleotides are oriented radially outwards. The orientation of these oligonucleotides can be either 5′ distal/3′ terminal in relation to the core, or 3′ distal/5′terminal in relation to the core, or laterally oriented around the core. In one embodiment one or a multiplicity of different oligonucleotides are present on the same surface of a single SNA. In all cases, at least 1 oligonucleotide is present on the surface but up to 10,000 can be present.

In some embodiments, an oligonucleotide disclosed herein is uniformly dispersed or suspended around a core, such as a liposomal core or a gold core. In some embodiments, the oligonucleotide is not uniformly dispersed or suspended around a core, such as a liposomal core or gold core.

In some embodiments, the core is a liposomal core comprising a plurality of lipids. In some embodiments, the liposomal core comprises one type of lipid. In some embodiments, the liposomal core comprises two to 100 types of lipids. In some embodiments, the liposomal core comprises two, three, four, five, six, seven, eight, nine, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more than 100 types of lipids.

In some embodiments, the lipids are selected from the group consisting of 1,2-dioleoyl-sn-glycero-3-phosphocholine (DOPC), sphingolipids, sphingosine, sphingosine phosphate, methylated sphingosines sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids, phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols, cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives. In some embodiments, the lipid is DOPC.

In some embodiments, the SNA is free of lipids, polymers or solid cores.

In some embodiments, the SNA comprises a neutral lipid. Non-limiting examples of a neutral lipid include 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), and 1,2-dihexadecanoyl-sn-glycero-3-phosphoethanolamine (DPPE), any related phosphatidylcholine or neutral lipids available from commercial vendors or known to one of ordinary skill in the art.

In some embodiments, the SNA is in a formulation. In some embodiments, the formulation comprises a solution comprising α,α-trehalose dehydrate (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of α,α-trehalose dehydrate), sodium phosphate (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of; or 1 mM, 5 mM, 10 mM 15 mM, 20 mM, 25 mM, 30 mM, 35 mM, 40 mM, 45 mM, 50 mM, more than 50 mM or any combination or ranges thereof of sodium phosphate, such as sodium phosphate monobasic, monohydrate or sodium phosphate dibasic, anhydrous), sodium chloride (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of; or 1 mM, 5 mM, 10 mM 15 mM, 20 mM, 25 mM, 30 mM, 35 mM, 40 mM, 45 mM, 50 mM, more than 50 mM or any combination or ranges thereof of sodium chloride), polysorbate 20 (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of polysorbate 20), sodium bicarbonate (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of; or 1 mM, 5 mM, 10 mM 15 mM, 20 mM, 25 mM, 30 mM, 35 mM, 40 mM, 45 mM, 50 mM, more than 50 mM or any combination or ranges thereof of sodium bicarbonate), sodium carbonate (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of; or 1 mM, 5 mM, 10 mM 15 mM, 20 mM, 25 mM, 30 mM, 35 mM, 40 mM, 45 mM, 50 mM, more than 50 mM or any combination or ranges thereof of sodium carbonate), histidine HCl (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of; or 1 mM, 5 mM, 10 mM 15 mM, 20 mM, 25 mM, 30 mM, 35 mM, 40 mM, 45 mM, 50 mM, more than 50 mM or any combination or ranges thereof of histidine HCl), and/or a sugar such as sucrose or dextrose (e.g., 0.001% 0.005%, 0.01%, 0.02%, 0.03%, 0.04%, 0.05%, 0.06%, 0.07%, 0.08%, 0.09%, 0.1%, 1.2%, 1.4%, 1.6%, 1.8%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, more than 50% or any combination or ranges thereof of a sugar, such as sucrose or dextrose).

In some embodiments, the formulation is at a pH between 4 and 10. In some embodiments the pH is 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more than 10. In some embodiments, the pH is adjusted with hydroxide and/or hydrochloric acid. In some embodiments, the formulation comprises a permeability enhancer to increase penetration of a SNA disclosed herein. In some embodiments, the permeability enhancer increases penetration of a SNA disclosed herein to the anterior chamber of the eye or the posterior chamber of the eye.

In some embodiments, the permeability enhancer is a surfactant, bile acid, chelating agent, or a preservative. In some embodiments, the permeability enhancer is a cyclodextrin. In some embodiments, the permeability enhancer increases chemical stability, increases bioavailability and/or decreases local irritation. Other permeability enhancers known to one of ordinary skill in the art, such as those disclosed in Davis et al. (Curr Opin Mol Ther (2004) 6(2):195-205), are also contemplated herein.

As used herein, “pharmaceutically acceptable salts” means physiologically and pharmaceutically acceptable salts of compounds that retain the desired biological activity of the parent compound and do not impart undesired toxicological effects thereto.

As used herein, “pharmaceutical composition” means a mixture of substances suitable for administering to a subject. For example, a pharmaceutical composition may comprise a SNA and a sterile aqueous solution. In some embodiments, a pharmaceutical composition shows activity in free uptake assays in certain cell lines.

For use in therapy, an effective amount of the SNAs or structures can be administered to a subject by any mode that delivers the SNAs to the desired cell. Administering pharmaceutical compositions may be accomplished by any means known to the skilled artisan. Routes of administration include but are not limited to oral, parenteral, intramuscular, intravenous, intrathecal, intravitreal, topical, subcutaneous, mucosal, intranasal, sublingual, intratracheal, inhalation, ocular, vaginal, dermal or rectal administration, and by direct injection.

The term “effective amount” refers to the amount of at least one SNA at dosages and for periods of time necessary to achieve the desired therapeutic result (e.g., improve, ameliorate, improve, reduce or stop the eye disease or eye disorder). In some embodiments, an effective amount of a SNA disclosed herein is considered as the amount sufficient to reduce a symptom of of the eye disease or eye disorder by at least 10%. An effective amount as used herein would also include an amount sufficient to prevent or delay the development of the eye disease or eye disorder, or reverse an eye disease or eye disorder. “Effective amount” also refers to the amount of SNA which exerts a beneficial effect on stopping the progression of the eye disease or eye disorder, or slowing the progression of the eye disease or eye disorder. The dosage administered, as single or multiple doses, to an individual will vary depending upon a variety of factors, including pharmacokinetic properties of the SNA, the route of administration, conditions and characteristics (sex, age, body weight, health, size) of subjects, extent of symptoms, concurrent treatments, frequency of treatment and the effect desired. An effective amount is also one in which any toxic or detrimental effects of the therapeutic agent are outweighed by the therapeutically beneficial effects. The effective amount in each individual case can be determined empirically by one of ordinary skill in the art according to established methods in the art and without undue experimentation.

In some embodiments, the ocular bioavailability of a SNA in a formulation described herein is 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 99%, or 100%.

The terms “oligonucleotide” and “nucleic acid” are used interchangeably to mean multiple nucleotides (i.e., molecules comprising a sugar (e.g., ribose or deoxyribose)) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis).

In some embodiments, an oligonucleotide disclosed herein comprises a modified nucleoside. In some embodiments, an oligonucleotide disclosed herein comprises or consists of one nucleotide with a 2′-modification or a 2′-substituent. In some embodiments, an oligonucleotide disclosed herein comprises or consists of two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides with a 2′-modification or a 2′-substituent. In some embodiments, the 2′-modification or 2′substituent is selected from the group consisting of: 2′-deoxy, 2′-deoxy-2′-fluoro, 2′-O-methyl, 2′-O-methoxyethyl (2′-O-MOE), 2′O-aminopropyl (2′-O-AP), 2′-O-dimethylaminoethyl (2′-O-DMAOE), 2′-dimethylaminopropyl (2′-O-DMAP), 2′-O-dimethylaminoethyloxyethyl(2′-O-DMAEOE), and 2′-O—N-methylacetamido (2′-O-NMA).

In some embodiments, the modified nucleoside comprises a modified sugar moiety. In some embodiments, the modified sugar moiety comprises a 2′-substituent. In some embodiments, the 2′-modification or 2′-substituent is selected from the group consisting of: 2′-deoxy, 2′-deoxy-2′-fluoro, 2′-O-methyl, 2′-O-methoxyethyl (2′-O-MOE), 2′O-aminopropyl (2′-O-AP), 2′-O-dimethylaminoethyl (2′-O-DMAOE), 2′-dimethylaminopropyl (2′-O-DMAP), 2′-O-dimethylaminoethyloxyethyl(2′-O-DMAEOE), and 2′-O—N-methylacetamido (2′-O-NMA). In some embodiments, the 2′-substituent is selected from the group consisting of: 2′-O-methyl (2′-OMe), 2′-fluoro (2′-F), and 2′-O-methoxy-ethyl (2′-MOE).

In some embodiments, the modified sugar moiety is a bicyclic sugar moiety. In some embodiments, the bicyclic sugar moiety is locked nucleic acid (LNA) or constrained ethyl nucleoside (cEt). In some embodiments, the modified sugar moiety comprises a sugar surrogate. In some embodiments, a sugar surrogate is a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids. In some embodiments, the sugar surrogate is a morpholino. In some embodiments, the sugar surrogate is a peptide nucleic acid (PNA).

In some embodiments, oligonucleotides comprise one or more type of modified sugar and/or unmodified sugar moiety arranged along the oligonucleotide or region thereof in a defined pattern or sugar motif. In some embodiments, the sugar motif is a sugar modification disclosed herein. In some embodiments, an oligonucleotide disclosed herein comprises or consists of a region having a fully modified sugar motif. In such embodiments, each nucleoside of the fully modified region of the modified oligonucleotide comprises a modified sugar moiety. In certain such embodiments, each nucleoside in the entire modified oligonucleotide comprises a modified sugar moiety. In some embodiments, modified oligonucleotides comprise or consist of a region having a fully modified sugar motif, wherein each nucleoside within the fully modified region comprises the same modified sugar moiety, referred to herein as a uniformly modified sugar motif. In some embodiments, a fully modified oligonucleotide is a uniformly modified oligonucleotide. In some embodiments, each nucleoside of a uniformly modified oligonucleotide comprises the same 2′-modification. In some embodiments, each nucleoside of a uniformly modified oligonucleotide comprises a 2′-O—(N-alkyl acetamide) group. In some embodiments, each nucleoside of a uniformly modified oligonucleotide comprises a 2′-O—(N-methyl acetamide) group.

In some embodiments, an oligonucleotide disclosed herein is modified at the sugar moiety, the phosphodiester linkage, and/or the base. As used herein, “sugar moieties” includes natural, unmodified sugars, including pentose, hexose, conformationally flexible sugars, conformationally locked sugars, arabinose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, oligonucleotides associated with the invention may only contain modified or unmodified sugar moieties, while in other instances, oligonucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH₂, NHR, NR₂,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose or arabinose.

In some embodiments, the oligonucleotide is a DNA oligonucleotide, a DNA-RNA hybrid oligonucleotide, or an RNA oligonucleotide. In some embodiments, the RNA oligonucleotide is an siRNA, miRNA, mRNA, non-coding RNA, or aptamer.

In some embodiments, the oligonucleotide is linked to the molecular species at the 3′ or 5′ terminus of the oligonucleotide through a linker, wherein the molecular species is adsorbed to the liposomal core of the SNA and the oligonucleotides forming the oligonucleotide shell extend radially from the core, and wherein the oligonucleotides forming the oligonucleotide shell comprise the entire SNA such that no other structural components are part of the SNA.

In some embodiments, the molecular species is attached to either or both ends of an oligonucleotide and/or at any internal position. In some embodiments, the molecular species is attached directly (e.g., covalently attached) to either or both ends of an oligonucleotide and/or at any internal position. In some embodiments, the molecular species is attached indirectly (e.g., non-covalently attached, etc.) to either or both ends of an oligonucleotide and/or at any internal position. In some embodiments, the molecular species is indirectly attached to either or both ends of an oligonucleotide and/or at any internal position through a linker. In some embodiments, a molecular species is attached to the 2′-position of a nucleoside of a modified oligonucleotide. In some embodiments, the molecular species is attached at the 3′ and/or 5′-end of the oligonucleotide. In some embodiments, the molecular species is attached near the 5′-end of the oligonucleotide. In some embodiments, the molecular species is attached near the 3′-end of the oligonucleotide.

In some embodiments, oligonucleotides are covalently attached to one or more molecular species. In some embodiments, molecular species modify one or more properties of the attached oligonucleotide, including but not limited to pharmacodynamics, pharmacokinetics, stability, binding, absorption, tissue distribution, cellular distribution, cellular uptake, charge and clearance. In some embodiments, molecular species impart a new property on the attached oligonucleotide, e.g., fluorophores or reporter groups that enable detection of the oligonucleotide. Certain molecular species and conjugate moieties have been described previously, for example: cholesterol (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBSLett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In some embodiments, the molecular species is an intercalator, reporter molecule, polyamine, polyamide, peptide, carbohydrate (e.g., GalNAc), vitamin moiety, polyethylene glycol, thioether, polyether, cholesterol, thiocholesterol, cholic acid moiety, folate, lipid, lipophilic group, phospholipid, biotin, phenazine, phenanthridine, anthraquinone, adamantane, acridine, fluoresceins, rhodamines, coumarins, fluorophores, or dye. In some embodiments, a molecular species comprises an active drug substance, for example, aspirin, warfarin, phenylbutazone, ibuprofen, suprofen, fen-bufen, ketoprofen, (<S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

A liposomal core as used herein refers to a centrally located core compartment formed by a component of the lipids or phospholipids that form a lipid bilayer. “Liposomes” are artificial, self closed vesicular structure of various sizes and structures, where one or several membranes encapsulate an aqueous core. Most typically liposome membranes are formed from lipid bilayers, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipid molecules. Each lipid molecule in a layer is oriented substantially parallel to adjacent lipid molecules in the lipid layer, and two layers that form a bilayer have the polar ends of their molecules exposed to the aqueous phase and the non-polar ends adjacent to each other. The central aqueous region of the liposomal core may be empty or filled fully or partially with water, an aqueous solution or emulsion, oligonucleotides, or other therapeutic or diagnostic agents.

The lipid-containing core can be constructed from a wide variety of lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, 1,2-dioleoyl-sn-glycero-3-phosphocholine (DOPC) and polyunsaturated sterols of different lengths, saturation states, and their derivatives.

### EXAMPLES

### Example 1. Spherical Nucleic Acids Exhibit Enhanced Distribution and Persistence Compared to Linear Oligonucleotides in Rat Eyes Following Intravitreal Administration

### Methods

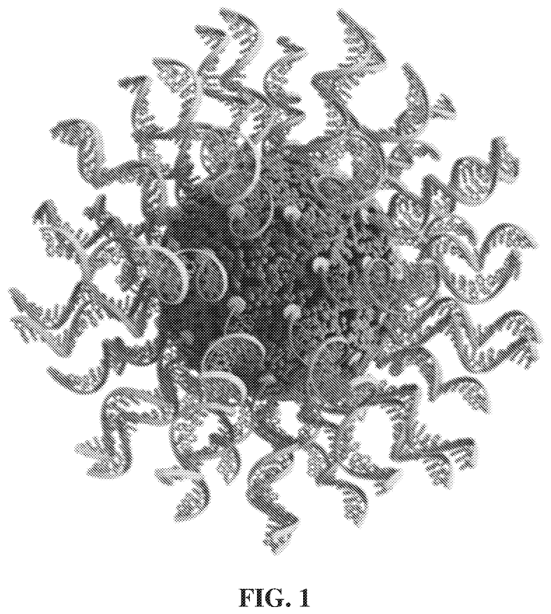

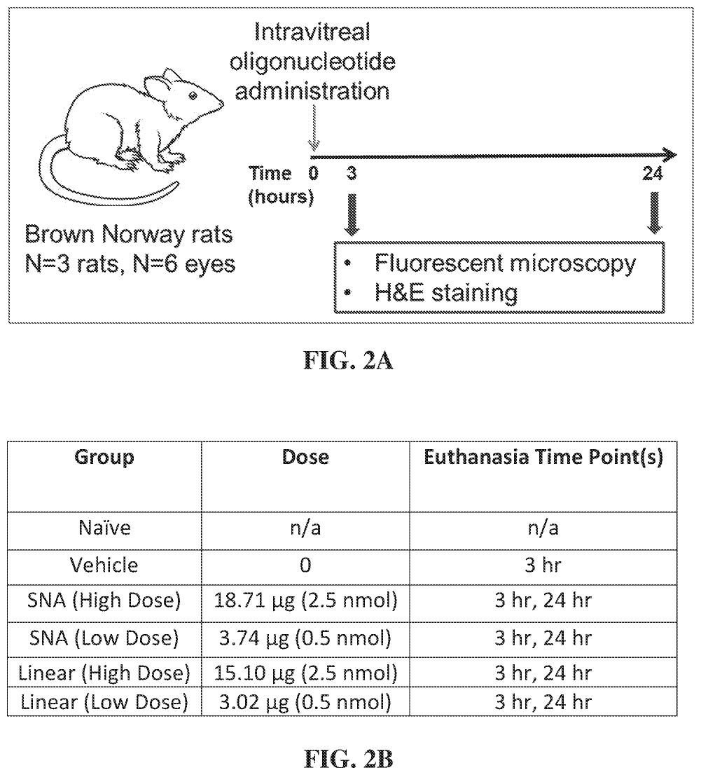

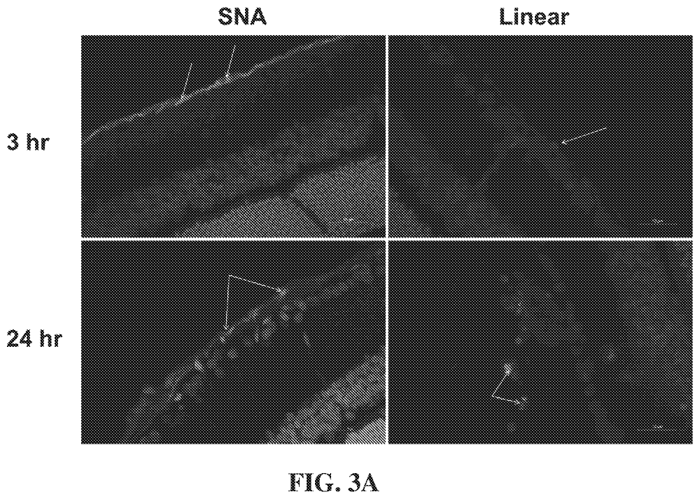

The biodistributions of SNAs and linear oligonucleotides in rat eyes following intravitreal injection were compared. SNA and linear oligonucleotides were synthesized with Cy5 fluorescent labels. Intravitreal injection (IVI) was administered bilaterally in male Brown Norway rats. There were three rats/six eyes per treatment group as shown in FIG. 2A. Equimolar amounts of Cy5-labeled oligonucleotides were delivered in either SNA or linear format at 5 μL per eye. As shown in FIG. 2B, the high dose was (500 μM)=2.5 nmol, the low dose was (100 μM)=0.5 nmol, and 5% dextrose was used as the vehicle. Whole eyes were collected at either 3 hours or 24 hours following IVI. Sister sections were used for H&E staining and fluorescent microscopy. Sections for fluorescent microscopy were stained with DAPI to indicate nuclei, and both DAPI and Cy5 (indicating oligonucleotides) were imaged. A schematic showing the structure of SNA is shown in FIG. 1.

### Results

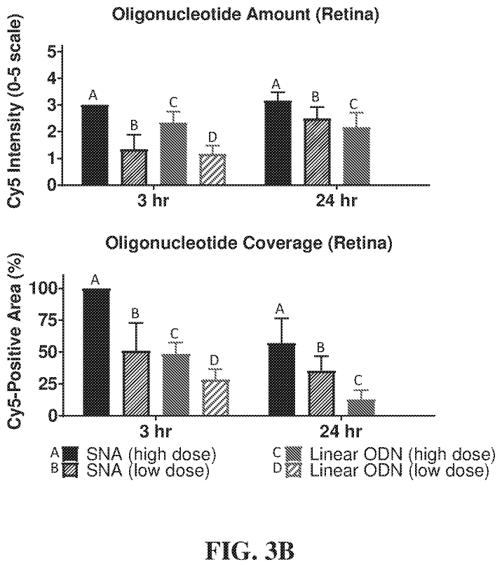

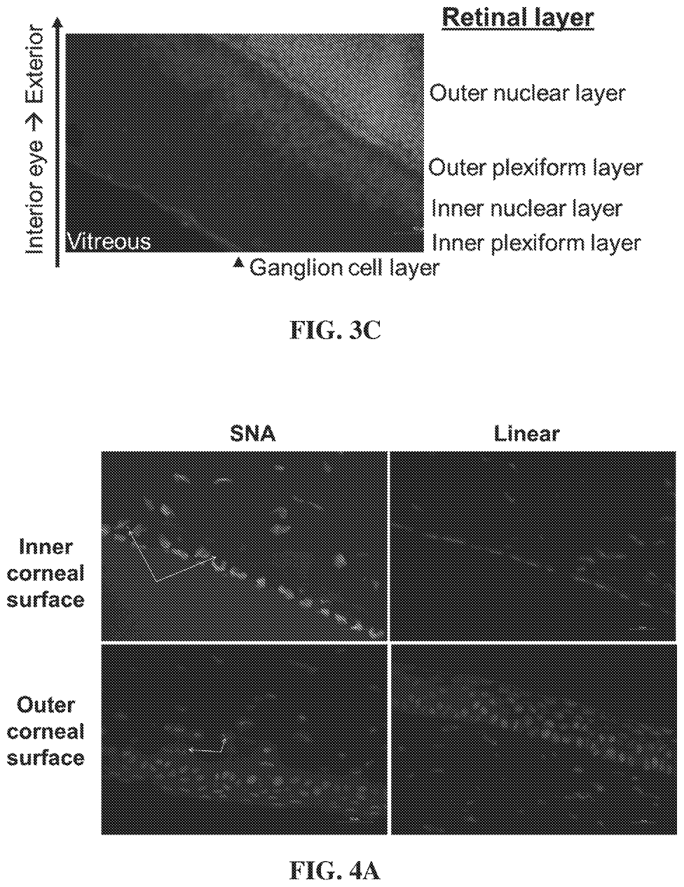

FIG. 3A shows representative images of the retina at 3 hours and 24 hours following IVI of high-dose (2.5 nmol) oligonucleotide. The arrows indicate intracellular signals which are located in ganglion cells. FIG. 3B is a graph showing a semi-quantitative assessment of oligonucleotide amount and retinal surface coverage. FIG. 3C is a representative image of SNA distribution through retinal layers 24 hours following IVI of the high-dose (2.5 nmol).

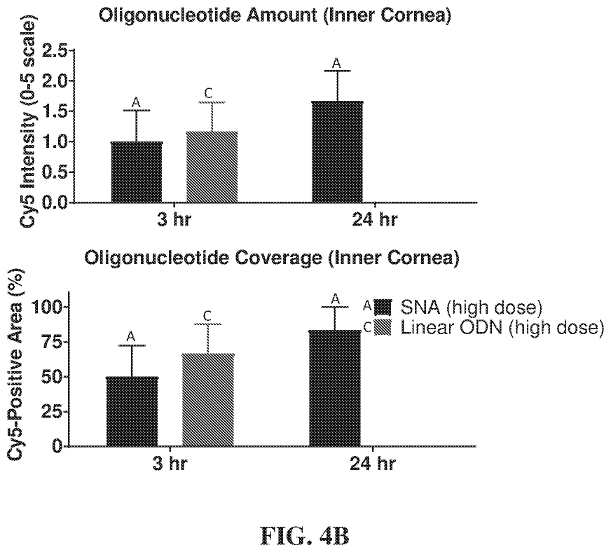

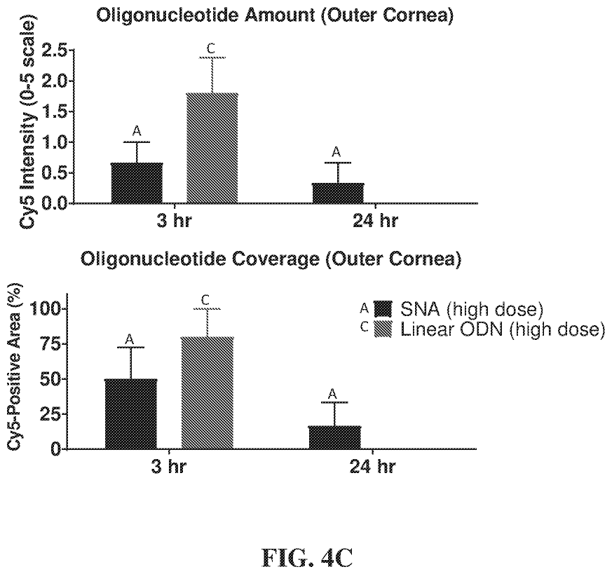

FIG. 4A shows representative images of corneal distribution and persistence 24 hours following IVI of high-dose (2.5 nmol) oligonucleotide. The arrows indicate intracellular signal. FIG. 4B is a graph showing a semi-quantitative assessment of oligonucleotide amount and surface coverage in the inner cornea after 3 hours and 24 hours. FIG. 4C is a graph showing a semi-quantitative assessment of oligonucleotide amount and surface coverage in the outer cornea after 3 hours and 24 hours, which shows the SNA format increases corneal distribution and persistence relative to the linear oligonucleotide.

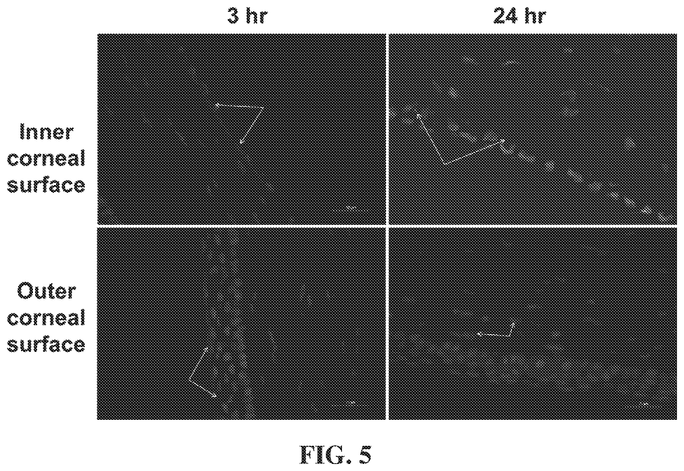

FIG. 5 shows representative images of the inner and outer corneal surfaces following IVI of high-dose (2.5 nmol) oligonucleotide in SNA format. The arrows indicate the intracellular signals which show the SNA signals increase over time.

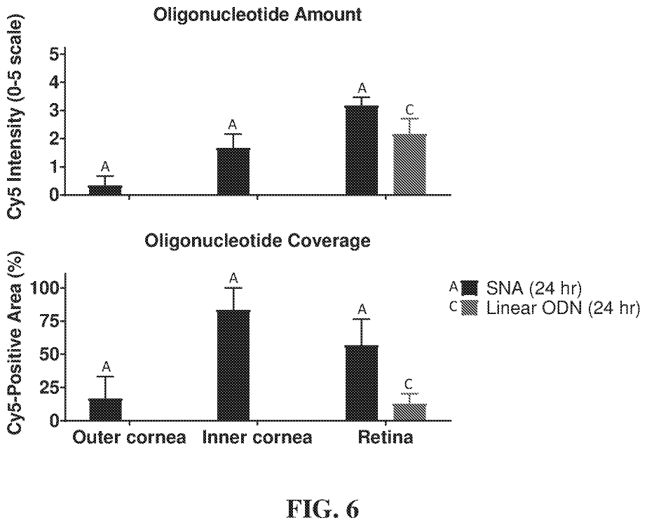

FIG. 6 shows graphs of a semi-quantitative assessment of oligonucleotide amounts and surface coverage 24 hours following an IVI of high dose (2.5 nmol) oligonucleotides in SNA and linear formats. The graphs show the SNA signal persists over time.

A summary of inflammation findings from the pathology report is shown in Table 1. Minimal to mild inflammation was observed in all groups except naïve animals. The inflammation was located in the sclera and sub-scleral tissue, and was composed of neutrophils and macrophages. Since the observed inflammation was located on one side of each eye, it was likely a result of the injection itself.

TABLE 1
Pathology report regarding inflammation
Inflammation severity and cell types

| Group | 3 hours | 24 hours |
|---|---|---|
| Naïve | None | n/a |
| Vehicle | Minimal, neutrophils | n/a |
| SNA (High Dose) | Minimal, neutrophils | Mild, neutrophils & |
| | | macrophages |
| SNA (Low Dose) | Minimal, neutrophils | Minimal, neutrophils |
| | | & macrophages |
| Linear (High Dose) | Minimal, neutrophils | Mild, neutrophils & |
| | | macrophages |
| Linear (Low Dose) | Minimal, neutrophils | Minimal, neutrophils |
| | | & macrophages |

### CONCLUSION

Following IVI of SNAs in rat eyes, SNAs distribute to both posterior and anterior ocular structures, exhibit higher distribution than linear oligonucleotides, persist longer compared to linear oligonucleotides, and do not cause inflammation in the eye. These data support the therapeutic potential of intravitreally delivered SNAs for treating eye diseases.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method of treating an eye disorder or eye disease in a subject, the method comprising administering to a subject through intravitreal injection an effective amount of a spherical nucleic acid (SNA) comprising oligonucleotides forming an oligonucleotide shell to treat the eye disorder or eye disease in the subject.

2. A method of increasing the persistence of an oligonucleotide in the eye of a subject, the method comprising administering to a subject through intravitreal injection a spherical nucleic acid (SNA) comprising oligonucleotides forming an oligonucleotide shell to increase the persistence of the oligonucleotide in the eye of the subject, wherein the persistence of the oligonucleotides in the SNA is increased relative to linear oligonucleotides that are not in a SNA.

3. A method of delivering an oligonucleotide to one or more regions in the eye of a subject, the method comprising administering to the subject through intravitreal injection a spherical nucleic acid (SNA) comprising oligonucleotides forming an oligonucleotide shell to reach one or more regions of the eye, wherein the one or more regions of the eye comprise the posterior segment or anterior segment of the eye of the subject.

4. The method of any one of claims 1-3, wherein the oligonucleotide is a TNF-α inhibitor, a receptor tyrosine kinase (RTK) inhibitor, a cyclooxygenase (COX) inhibitor, an interleukin 1 beta (IL1β) inhibitor, a beta-2 adrenergic receptor (ADRB2) inhibitor, a connective tissue growth factor (CTGF) inhibitor or a vascular endothelial growth factor (VEGF) inhibitor, a platelet-derived growth factor subunit A (PDGFA) inhibitor, a platelet-derived growth factor subunit B (PDGFB) inhibitor, a platelet-derived growth factor subunit C (PDGFC) inhibitor, a platelet-derived growth factor subunit D (PDGFD) inhibitor, a platelet-derived growth factor receptor alpha (PDGFRA) inhibitor, a platelet-derived growth factor receptor beta (PDGFRB) inhibitor, a platelet-derived growth factor receptor like (PDGFRL) inhibitor, a vascular endothelial growth factor A (VEGFA) inhibitor, a vascular endothelial growth factor B (VEGFB) inhibitor, a vascular endothelial growth factor C (VEGFC) inhibitor, a vascular endothelial growth factor D (VEGFD) inhibitor, a vascular endothelial growth factor receptor-1 inhibitor, a vascular endothelial growth factor receptor-2 inhibitor, a vascular endothelial growth factor receptor-3 inhibitor, a beta-2 adrenergic receptor inhibitor, a connective tissue growth factor inhibitor, an interleukin-1β inhibitor, an interleukin-1 receptor-1 inhibitor, an interleukin-1 receptor-2 inhibitor, or an interleukin-1 receptor-3 inhibitor.

5. The method of any one of claims 1-4, wherein the oligonucleotide is an antisense oligonucleotide.

6. The method of claim 4, wherein the TNF-α inhibitor is a TNF-α antisense oligonucleotide.

7. The method of claim 6, wherein the TNF-α antisense oligonucleotide is 8-40 nucleotides in length.

8. The method of any one of claims 1-5, wherein the oligonucleotide is not a TNF-α antisense oligonucleotide.

9. The method of any one of claims 1-4, wherein the oligonucleotide is a DNA oligonucleotide, a DNA-RNA hybrid oligonucleotide, or an RNA oligonucleotide.

10. The method of claim 9, wherein the RNA oligonucleotide is an siRNA, miRNA, mRNA, non-coding RNA, or aptamer.

11. The method of any one of claims 1-10, wherein the oligonucleotides comprise a phosphorothioate modification.

12. The method of any one of claims 1-11, wherein the SNA is administered to the subject at a dose of between 2 μg and 1 mg.

13. The method of any one of claims 1-11, wherein the SNA is administered to the subject at a dose of between 2 μg and 20 μg.

14. The method of any one of claims 1-13, wherein the SNA is delivered to the retina or cornea of the subject.

15. The method of any one of claims 1-14, wherein the eye disorder or eye disease is associated with ocular angiogenesis, ocular neovascularization, retinal edema, ocular hypertension, elevated intraocular pressure, retinal ischemia, posterior segment neovascularization, age-related macular degeneration, inflammation, macular edema, uveitis, dry eye, neovascular glaucoma, glaucoma, scleritis, diabetic retinopathy, retinitis pigmentosa, optic nerve injury, retinopathy of prematurity, retinal ganglion degeneration, macular degeneration, hereditary optic neuropathy, metabolic optic neuropathy, acute ischemic optic neuropathy, commotio retinae, retinal detachment, retinal tears, retinal holes, iatrogenic retinopathy, myopia, conjunctivitis or eye cancer.

16. The method of any one of claims 1-15, wherein the subject is a mammal.

17. The method of any one of claims 1-15, wherein the subject is human.

18. The method of any one of claims 1-17, wherein the oligonucleotide is linked to a molecular species at the 3' or 5′ terminus of the oligonucleotide through a linker.

19. The method of claim 18, wherein the molecular species is linked to the oligonucleotide at the 3′ end of the oligonucleotide.

20. The method of claim 18 or 19, wherein the molecular species is a hydrophobic group.

21. The method of claim 20, wherein the hydrophobic group is selected from the group consisting of cholesterol, a cholesteryl, a modified cholesteryl residue, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid, taurocholic acid, deoxycholate, oleyl litocholic acid, oleoyl cholenic acid, glycolipids, phospholipids, sphingolipids, isoprenoids, vitamins, saturated fatty acid, unsaturated fatty acid, fatty acid ester, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dye, Hoechst 33258 dye, psoralen and ibuprofen.

22. The method of claim 20, wherein the hydrophobic group is selected from the group consisting of a steroid, vitamin E, triglyceride, Cy3 and Cy5.

23. The method of claim 20, wherein the hydrophobic group is cholesterol.

24. The method of any one of claims 1-23, wherein the SNA comprises a core and wherein the plurality of oligonucleotides are linked to the exterior of the core.

25. The method of claim 24, wherein the core is a liposomal core comprising a plurality of lipids.

26. The method of claim 25, wherein the liposomal core comprises one type of lipid.

27. The method of claim 25, wherein the liposomal core comprises two to 10 types of lipids.

28. The method of any one of claims 24-27, wherein the lipids are selected from the group consisting of 1,2-dioleoyl-sn-glycero-3-phosphocholine (DOPC), sphingolipids, sphingosine, sphingosine phosphate, methylated sphingosines sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids, phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols, cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

29. The method of any one of claims 24-28, wherein the lipids consist of DOPC.

30. The method of any one of claims 24-29, wherein the oligonucleotides are indirectly linked to the core through a linker.

31. The method of any one of claims 24-29, wherein the oligonucleotides are indirectly linked to the core through more than one linker.

32. The method of any one of claims 24-29, wherein the oligonucleotides are directly linked to the core.

33. The method of claim 30 or 31, wherein the linker is a non-nucleotidic linker.

34. The method of any one of claim 30, 31 or 33, wherein the linker is selected from the group consisting of abasic residues (dSpacer), oligoethyleneglycol, triethyleneglycol, hexaethylenegylcol, alkane-diol, or butanediol.

35. The method of any one of claim 30, 31, 33 or 34, wherein the linker is a double linker or a triple linker.

36. The method of claim 35, wherein the double linker is two oligoethyleneglycols.

37. The method of claim 36, wherein the two oligoethyleneglycols are hexaethylenegylcols.

38. The method of any one of claims 35-37, wherein the double linker or the triple linker is linked in between each single linker by a phosphodiester, phosphorothioate, methylphosphonate, or amide linkage.

39. The method of any one of claims 1-38, wherein the SNA comprises two to 1,000 oligonucleotides.

40. The method of any one of claims 1-38, wherein the SNA comprises 10 to 40 oligonucleotides.

41. The method of any one of claims 1-38, wherein the SNA comprises 25 to 35 oligonucleotides.

42. The method of any one of claims 1-38, wherein the SNA comprises 30 oligonucleotides.

43. The method of any one of claims 1-42, wherein the SNA is 10 to 40 nm in diameter.

44. The method of any one of claims 1-42, wherein the SNA is 30 nm in diameter.

45. The method of any one of claims 25-44, wherein the liposomal core is 10 to 40 nm in diameter.

46. The method of any one of claims 25-45, wherein the liposomal core is 20 nm in diameter.

47. The method of any one of claims 1-46, wherein the SNA is delivered in a formulation.

48. The method of claim 47, wherein the formulation comprises 5% dextrose.

49. The method of any one of claims 25-48, wherein the oligonucleotide is linked to the molecular species at the 3′ or 5′ terminus of the oligonucleotide through a linker, wherein the molecular species is adsorbed to the liposomal core of the SNA and the oligonucleotides forming the oligonucleotide shell extend radially from the core, and wherein the oligonucleotides forming the oligonucleotide shell comprise the entire SNA such that no other structural components are part of the SNA.

50. The method of any one of claims 4-49, wherein the antisense oligonucleotide inhibits the expression of a gene in the eye of the subject, wherein the expression of the gene is inhibited by between 50% and 99% relative to a baseline level of expression of the gene in the eye of the subject.

51. The method of any one of claims 4-49, wherein the antisense oligonucleotide inhibits the expression of a gene in the eye of the subject, wherein the expression of the gene is inhibited by between 50% and 99% relative to the level of inhibition of the gene in the eye of the subject with the corresponding linear antisense oligonucleotide that is not in a SNA.

52. The method of any one of claims 4-51, wherein the antisense oligonucleotide comprises a modified nucleoside.

53. The method of claim 52, wherein the modified nucleoside comprises a modified sugar moiety.

54. The method of claim 53, wherein the modified sugar moiety comprises a 2′-substituent.

55. The method of claim 54, wherein the 2′-substituent is selected from the group consisting of: 2′-O-methyl (2′-OMe), 2′-fluoro (2′-F), and 2′-O-methoxy-ethyl (2′-MOE).

56. The method of claim 54, wherein the 2′-substituent is 2′-MOE.

57. The method of any one of claims 53 to 56, wherein the modified sugar moiety is a bicyclic sugar moiety.

58. The method of claim 57, wherein the bicyclic sugar moiety is locked nucleic acid (LNA) or constrained ethyl nucleoside (cEt).

59. The method of claim 53, wherein the modified sugar moiety comprises a sugar surrogate.

60. The method of claim 59, wherein the sugar surrogate is a morpholino or peptide nucleic acid.

61. The method of any one of claims 4-60, wherein the antisense oligonucleotide comprises a backbone that comprises a phosphorothioate internucleoside linkage.

62. The method of claim 61, wherein the phosphorothioate internucleoside linkage has a (Sp) stereochemical configuration.

63. The method of claim 61, wherein the phosphorothioate internucleoside linkage has a (Rp) stereochemical configuration.

64. The method of any one of claims 4-60, wherein the antisense oligonucleotide comprises a backbone that consists of phosphorothioate internucleoside linkages.

65. The method of claim 64, wherein the phosphorothioate internucleoside linkages have a (Sp) stereochemical configurations.

66. The method of claim 64, wherein the phosphorothioate internucleoside linkages have a (Rp) stereochemical configurations.

67. The method of claim 61 or 64, wherein the phosphorothioate internucleoside linkages have the same stereochemical configuration.

68. The method of claim 61 or 64, wherein the phosphorothioate internucleoside linkages have different stereochemical configurations.

69. The method of claim 61 or 64, wherein two to 30 of the phosphorothioate internucleoside linkages have the same stereochemical configuration.

70. The method of any one of claims 4-60, wherein the antisense oligonucleotide consists of two to 30 phosphorothioate internucleoside linkages.
